(19) United States
(12) Patent Application Publication
(10) Pub. No.: US 2024/0254123 A1
(43) Pub. Date: Aug. 1, 2024

(54) PYRIDOPYRIMIDINE DERIVATIVES USEFUL AS WEE1 KINASE INHIBITORS

(71) Applicant: APREA THERAPEUTICS, INC., Doylestown, PA (US)

(72) Inventors: Oren GILAD, Doylestown, PA (US); Joseph VACCA, Telford, PA (US)

(21) Appl. No.: 18/565,598

(22) PCT Filed: Jun. 3, 2022
(86) PCT No.: PCT/US2022/032203

§ 371 Date: Nov. 30, 2023

Related U.S. Application Data
(60) Provisional application No. 63/196,744, filed on Jun. 4, 2021.

(51) Int. Cl. C07D 471/04; A61K 31/519

(52) U.S. Cl. CPC C07D 471/04; A61K 31/519

(57) ABSTRACT

The disclosure provides compounds, or pharmaceutically acceptable salts thereof, and methods of using these compounds to inhibit Wee1 kinase and treat, for example, cancer in a subject. Claimed compounds include compounds (I).

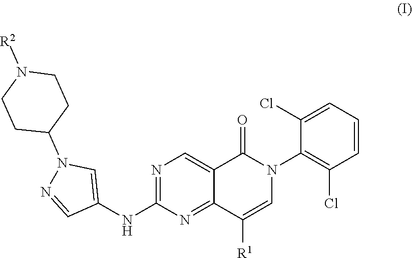

### CROSS-REFERENCE TO RELATED APPLICATIONS

This application claims priority to U.S. Provisional Patent Application No. 63/196,744, filed Jun. 4, 2021, the contents of which are incorporated by reference herein.

### FIELD OF THE INVENTION

This invention relates to compounds, and methods of use of these compounds, that are receptor protein tyrosine kinase inhibitors. In particular, it relates to the use of such compounds in the treatment or prevention of one or more conditions in which the functional effects of one or more kinases are elevated, such as cancer.

### BACKGROUND

Wee1 belongs to a family of protein kinases involved in the terminal phosphorylation and inactivation of cyclin-dependent kinase 1-bound cyclin B, resulting in G cell cycle arrest in response to DNA damage. Wee1 was first identified in fission yeast, where Wee1 deficiency resulted in premature mitotic entry and replication of smaller-sized yeast. It is the major kinase responsible for the inhibitory phosphorylation of the tyrosine. Wee1 is a tyrosine kinase that phosphorylates and inactivates Cdc2 and is involved in G checkpoint signaling. More particularly, Wee1 is involved in G₂-M checkpoint signaling. Because p53 is a key regulator in the G checkpoint, p53-deficient tumors rely only on the G checkpoint after DNA damage. More particularly, because p53 is a key regulator in the G₁-S checkpoint, p53-deficient tumors rely only on the G₂-M checkpoint after DNA damage. Hence, such tumors are selectively sensitized to DNA-damaging agents by Wee1 inhibition.

Before cells undergo mitosis, they progress through a tightly controlled cascade of G₁-S, intra-S, and G₂-M checkpoints. Wee1 kinase has emerged as a key G₂-M checkpoint regulator. This tyrosine kinase negatively regulates entry into mitosis by catalyzing an inhibitory phosphorylation of Cdc2 (the human homolog of cyclin-dependent kinase 1 (CDK1) on tyrosine-15 (Y15). This results in inactivation of the Cdc2/cyclin B complex, which arrests cells in G₂-M, allowing for DNA repair. Such inhibition also occurs through Chk1-mediated inhibition of Cdc25 phosphatases, which remove the inhibitory phosphorylation on Cdc2. Thus, entry into mitosis rests on a balance between the opposing activities of Wee1 and Chk1/Cdc25. Wee1 inhibition is thus expected to abrogate G₂-M arrest and propel cells into premature mitosis, a hypothesis confirmed by studies documenting that Wee1 inhibition by either small molecule inhibitors or small interference RNA leads to premature entry into mitosis and consequent cell death through mitotic catastrophe or apoptosis. (S. Muller, J. Clinical. Oncology, 2015).

Wee1 is highly expressed in several cancer types, including hepatocellular carcinoma, breast cancers, cervical cancers, lung cancers, squamous cell carcinoma, diffuse intrinsic pontine glioma (DIPG), glioblastoma, medulloblastoma, leukemia, melanoma, and ovarian cancers. (P. Reigan et al., Trends in Pharmacol. Sci., 2016).

Since there are few Wee1 inhibitors in clinical development, there is a need to improve Wee1 inhibitor selectivity and the properties of the inhibitors to permit targeting of specific cancer types, since inhibition of Wee1 activity can selectively promote the death of cancer cells with defective cell cycle checkpoints; at the same time, has little effect on normal cells with normal cell cycle checkpoints. Therefore, Wee1 inhibitors may be used as targeted drugs for the treatment of cancer and other cell proliferation disorders.

The disclosure provides compounds for use for treating, preventing, or managing one or more conditions in which the functional effects of one or more kinases are elevated, such as cancer, with the compound(s), or a pharmaceutically acceptable salts or prodrug thereof, as disclosed herein, and methods of using these compounds to inhibit, for example, Wee1 kinase and treat, prevent, or manage, for example, cancer in a subject.

All references cited herein are incorporated herein by reference in their entireties.

### BRIEF SUMMARY

The disclosure provides a compound for use for treating, preventing or managing a of one or more conditions in which the functional effects of one or more kinases are elevated, such as cancer, with the compound(s), or a pharmaceutically acceptable salts or prodrug thereof, as disclosed herein, and methods of using these compounds to inhibit, for example, Wee1 kinase and treat, prevent, or manage, for example, cancer in a subject.

The disclosure provides a compound having the structure of Formula I.

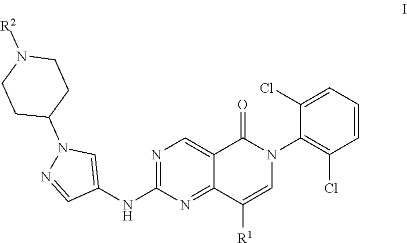

wherein: R¹ is halo, C₁₋₆ alkyl, C₃₋₈ cycloalkyl or C₂₋₆ alkenyl; and R² is H, C₁₋₆ alkyl, or C₃₋₈ cycloalkyl; or a pharmaceutically acceptable salt thereof. The disclosure provides a compound wherein R¹ is halo. The disclosure provides a compound wherein R¹ is chloro, bromo, fluoro, or iodo. The disclosure provides a compound wherein R¹ is chloro or bromo. The disclosure provides a compound wherein R¹ is C₁₋₄ alkyl. The disclosure provides a compound wherein R¹ is methyl, ethyl, n-propyl or i-propyl. The disclosure provides a compound wherein R¹ is C₃₋₇ cycloalkyl. The disclosure provides a compound wherein R¹ is cyclopropyl. The disclosure provides a compound wherein R¹ is C₁₋₄ alkenyl. The disclosure provides a compound wherein R¹ is vinyl or isopropenyl. The disclosure provides a compound wherein R² is C₁₋₆alkyl. The disclosure provides a compound wherein R² is methyl, ethyl, propyl, butyl, pentyl, or hexyl. The disclosure provides a compound wherein R² is methyl, ethyl, n-propyl, or i-propyl. The disclosure provides a compound wherein R² is C₃₋₈cycloalkyl. The disclosure provides a compound wherein R² is cyclopropyl, cyclobutyl, cyclopentyl, or cyclohexyl. The disclosure provides a compound wherein R² is cyclopropyl.

The disclosure provides a compound having the structure of Formula II:

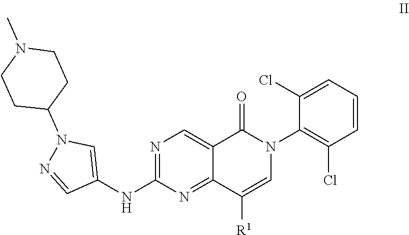

or a pharmaceutically acceptable salt thereof.

The disclosure provides a compound that is:

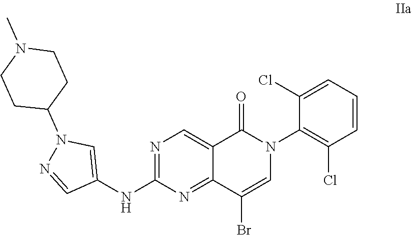 [SMILES: CN1CCC(n2cc(Nc3ncc4c(=O)n(-c5c(Cl)cccc5Cl)cc(Br)c4n3)cn2)CC1]

or a pharmaceutically acceptable salt thereof.

The disclosure provides a compound having the structure of Formula III:

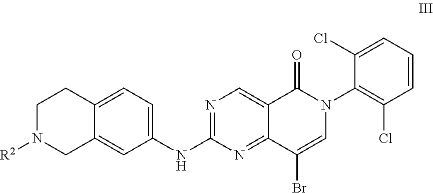

wherein: R² is H, C₁₋₆alkyl, or C₃₋₈ cycloalkyl, or a pharmaceutically acceptable salt thereof. The disclosure provides a compound wherein R² is H. The disclosure provides a compound wherein R² is C₁₋₆alkyl. The disclosure provides a compound wherein R² is methyl.

The disclosure provides a compound having the structure of Formula IV:

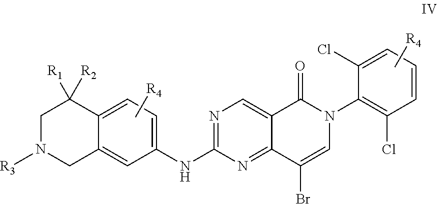

wherein R1, R2 is H; Methyl; Ethyl; or Propyl; optionally wherein R1 and R2 are connected to form a 3-6 membered ring; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

The disclosure provides a compound having the structure of Formula V:

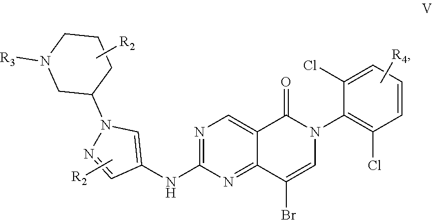

wherein R2 is H; Methyl; Ethyl; or Propyl; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

The disclosure provides a compound having the structure of Formula VI:

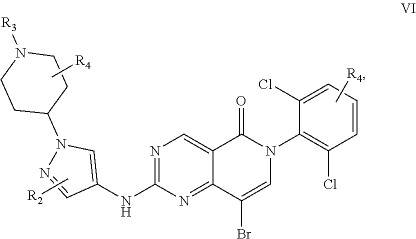

wherein R2 is H; Methyl; Ethyl; or Propyl; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

The disclosure also provides compounds of formula VIa or a pharmaceutically acceptable salt thereof:

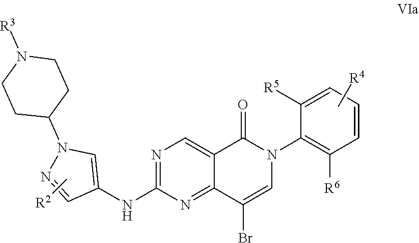

wherein, R²-R⁶ are defined herein.

The disclosure further provides compounds of Formula VII or a pharmaceutically acceptable salt thereof:

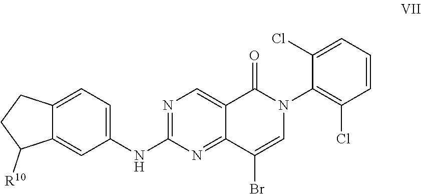

wherein R¹⁰ is defined herein.

The disclosure provides a pharmaceutical composition comprising one or more compounds as disclosed herein and one or more pharmaceutically acceptable excipient. The disclosure provides a method for inhibiting Wee1 in a patient in need of such treatment, comprising administering the compound or composition as disclosed herein to the patient. The disclosure provides a method of treating cancer in a patient need of such treatment, comprising administering to the patient the compound as disclosed herein to the patient. The disclosure provides a method, wherein the cancer is adrenocortical carcinoma, an AIDS-related cancer (such as an AIDS-related lymphoma), anal cancer, appendix cancer, astrocytomas, atypical teratoid/rhabdoid tumor, basal cell carcinoma, bile duct cancer, bladder cancer, bone cancer, brain cancer (such as glioblastoma), breast cancer, bronchial tumor, cancer of unknown primary site such as carcinoma of unknown primary site, carcinoid tumor, castration-resistant prostate cancer, central nervous system cancer (such as central nervous system atypical teratoid/rhabdoid tumor, central nervous system embryonal tumors, central nervous system lymphoma, primary central nervous system lymphoma), cervical cancer, chordoma, chondrosarcoma, chronic myeloproliferative disorders, colon cancer, colorectal cancer, craniopharyngioma, desmoplastic round cell tumor, diffuse large B-cell lymphoma, endometrial cancer, ependymoblastoma, ependymoma, esophageal cancer, Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor, germ cell tumor, gestational trophoblastic tumor, glioma, head cancer, hepatocellular (liver) cancer, high grade prostate cancer, histiocytosis, hypopharyngeal cancer, Kaposi sarcoma, kidney (renal) cancer, Langerhans cell histiocytosis, laryngeal cancer, leptomeningeal disease, lip cancer, low grade prostate cancer, leukemia (such as chronic lymphocytic leukemia, chronic myelogenous leukemia, hairy cell leukemia,), lung cancer, lymphoma (such as Burkitt lymphoma, central nervous system lymphoma, T-Cell lymphoma such as cutaneous T-Cell lymphoma, Hodgkin's lymphoma, Non-Hodgkin's lymphoma, lymphoplasmacytic lymphoma), medium grade prostate cancer, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, multiple myeloma/plasma cell neoplasm, mycosis fungoides, myelodysplastic syndromes, myelodysplastic/myeloproliferative neoplasms, myeloproliferative disorder, nasal cavity or paranasal sinus cancer, nasopharyngeal cancer, neck cancer, neuroblastoma, ocular cancer, ocular melanoma, oral cancer, oropharyngeal cancer, oral cavity cancer, osteosarcoma or malignant fibrous histiocytoma of bone, osteosarcoma or malignant fibrous histiocytoma, ovarian cancer, ovarian germ cell tumor, ovarian epithelial cancer, ovarian low malignant potential tumor, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, pregnancy cancer, prostate cancer, rectal cancer, renal pelvis cancer, respiratory tract carcinoma involving the NUT gene on chromosome 15, retinoblastoma, rhabdomyosarcoma, salivary gland cancer, sarcoma, Sezary syndrome, skin cancer, skin carcinoma, small intestine cancer, soft tissue sarcoma, spinal cord tumor, squamous cell carcinoma, squamous neck cancer with occult primary, supratentorial primitive neuroectodermal tumors, testicular cancer, throat cancer, thymoma or thymic carcinoma, thyroid cancer, transitional cell cancer of the renal pelvis or ureter, unusual cancers of childhood, ureter cancer, urethral cancer, uterine sarcoma, vaginal cancer, vulvar cancer, Waldenstrom's macroglobulinemia (lymphoplasmacytic lymphoma), Wilm's tumor, or women's cancer.

The disclosure provides a method wherein the cancer is breast cancer, prostate cancer, pancreatic cancer, lung cancer, colorectal cancer, ovarian cancer, liver cancer, melanoma, renal cancer, a central nervous system cancer, brain cancer such as glioblastoma, a leukemia, or a lymphoma. The disclosure provides a method wherein the compound is administered in combination with at least one additional therapeutic agent. The disclosure provides a method wherein the at least one additional therapeutic agent is a chemotherapeutic. The disclosure provides a method wherein said chemotherapeutic agent is selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, campath, panitumumab, metazotuzumab, navuzumab, pymzumab, remoluzumab, bevacizumab, partuzumab, trastuzumab, cetuximab, obinutuzumab, olfamzumab, rituximab, alemtuzumab, tiemuzumab, toximab, bentuximab, daremuzumab, errotuzumab, T-DM1, ofatumumab, dinutuximab, blinatumomab, ipilimma, avastin, trastuzumab, rituximab, imatinib, gefitinib, erlotinib, osimertinib, afatinib, ceritinib, aletinib, crizotinib, erlotinib, lapatinib, sorafenib, sunitinib, nilotinib, dasatinib, pazopanib, temsirolimus, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, ixazomib, vismodegib, sonidegib, denosumab, thalidomide, lenalidomide, venetoclax, aldesleukin (recombinant human interleukin-2), sipueucel-T (prostate cancer therapeutic vaccine), palbociclib, olaparib, niraparib, rucaparib, talazoparib, and combinations thereof.

The disclosure provides a method for reducing the activity of a kinase encoded by the gene WEE1, the method comprising contacting the kinase with an inhibitory amount of a compound as disclosed herein. The disclosure provides a method wherein the method is carried out in vitro. The disclosure provides a method wherein the method is carried out in a subject. The disclosure provides a method for treating or preventing Wee1-mediated disease in a subject in need thereof comprising administering the subject an effective amount of a compound as disclosed herein, or a pharmaceutically acceptable salt or prodrug thereof, or a pharmaceutical composition thereof. The disclosure provides a method wherein the disease is a cancer selected from adrenocortical carcinoma, an AIDS-related cancer (such as an AIDS-related lymphoma), anal cancer, appendix cancer, astrocytomas, atypical teratoid/rhabdoid tumor, basal cell carcinoma, bile duct cancer, bladder cancer, bone cancer, brain cancer (such as glioblastoma), breast cancer, bronchial tumor, cancer of unknown primary site such as carcinoma of unknown primary site, carcinoid tumor, castration-resistant prostate cancer, central nervous system cancer (such as central nervous system atypical teratoid/rhabdoid tumor, central nervous system embryonal tumors, central nervous system lymphoma, primary central nervous system lymphoma), cervical cancer, chordoma, chondrosarcoma, chronic myeloproliferative disorders, colon cancer, colorectal cancer, craniopharyngioma, desmoplastic round cell tumor, diffuse large B-cell lymphoma, endometrial cancer, ependymoblastoma, ependymoma, esophageal cancer, Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor, germ cell tumor, gestational trophoblastic tumor, glioma, head cancer, hepatocellular (liver) cancer, high grade prostate cancer, histiocytosis, hypopharyngeal cancer, Kaposi sarcoma, kidney (renal) cancer, Langerhans cell histiocytosis, laryngeal cancer, leptomeningeal disease, lip cancer, low grade prostate cancer, leukemia (such as chronic lymphocytic leukemia, chronic myelogenous leukemia, hairy cell leukemia,), lung cancer, lymphoma (such as Burkitt lymphoma, central nervous system lymphoma, T-Cell lymphoma such as cutaneous T-Cell lymphoma, Hodgkin's lymphoma, Non-Hodgkin's lymphoma, lymphoplasmacytic lymphoma), medium grade prostate cancer, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, multiple myeloma/plasma cell neoplasm, mycosis fungoides, myelodysplastic syndromes, myelodysplastic/myeloproliferative neoplasms, myeloproliferative disorder, nasal cavity or paranasal sinus cancer, nasopharyngeal cancer, neck cancer, neuroblastoma, ocular cancer, ocular melanoma, oral cancer, oropharyngeal cancer, oral cavity cancer, osteosarcoma or malignant fibrous histiocytoma of bone, osteosarcoma or malignant fibrous histiocytoma, ovarian cancer, ovarian germ cell tumor, ovarian epithelial cancer, ovarian low malignant potential tumor, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, pregnancy cancer, prostate cancer, rectal cancer, renal pelvis cancer, respiratory tract carcinoma involving the NUT gene on chromosome 15, retinoblastoma, rhabdomyosarcoma, salivary gland cancer, sarcoma, Sezary syndrome, skin cancer, skin carcinoma, small intestine cancer, soft tissue sarcoma, spinal cord tumor, squamous cell carcinoma, squamous neck cancer with occult primary, supratentorial primitive neuroectodermal tumors, testicular cancer, throat cancer, thymoma or thymic carcinoma, thyroid cancer, transitional cell cancer of the renal pelvis or ureter, unusual cancers of childhood, ureter cancer, urethral cancer, uterine sarcoma, vaginal cancer, vulvar cancer, Waldenstrom's macroglobulinemia (lymphoplasmacytic lymphoma), Wilm's tumor, or women's cancer.

The disclosure provides for the use of the compounds and compositions of the disclosure for the production of a medicament for preventing and/or treating the indications as set forth herein.

In accordance with a further embodiment, the present disclosure provides for the use of the compounds and pharmaceutical compositions described herein, in an amount effective for use in a medicament, and most preferably for use as a medicament for treating a disease or disorder, for example, as set forth herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “about” when used in conjunction with a stated numerical value or range has the meaning reasonably ascribed to it by a person skilled in the art, i.e. denoting somewhat more or somewhat less than the stated value or range.

### Active Agent

In one aspect, provided is a compound, or a pharmaceutically acceptable salts or prodrug of, having the chemical structure of formula I.

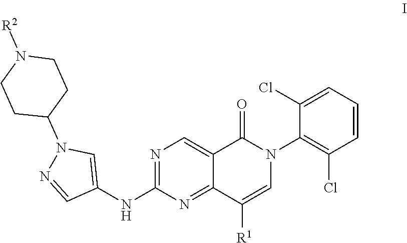

wherein: R¹ is halo, C₁₋₆alkyl, C₃₋₈cycloalkyl or C₂₋₆alkenyl; and R² is H, C₁₋₆alkyl, or C₃₋₈cycloalkyl; or a pharmaceutically acceptable salts thereof, and methods of using these compounds to inhibit WEE1 kinase and treat cancer in a subject. In further embodiments, R¹ is halo. In further embodiments, R¹ is chloro, bromo, fluoro, or iodo. In further embodiments, R¹ is chloro or bromo. In further embodiments, R¹ is C₁₋₄alkyl. In further embodiments, R¹ is methyl, ethyl, n-propyl or i-propyl. In other embodiments, R¹ is methyl. In further embodiments, R¹ is C₃₋₇cycloalkyl. In further embodiments, R¹ is cyclopropyl. In further embodiments, R¹ is C₁₋₄alkenyl. In further embodiments, R¹ is vinyl or isopropenyl. In yet other embodiments, R¹ is vinyl. In still further embodiments, R¹ is isopropenyl. In other embodiments, R² is H. In further embodiments, R² is C₁₋₆alkyl. In further embodiments, R² is methyl, ethyl, propyl, butyl, pentyl, or hexyl. In further embodiments, R² is methyl, ethyl, n-propyl, or i-propyl. In further embodiments, R² is C₃₋₈cycloalkyl. In further embodiments, R² is cyclopropyl, cyclobutyl, cyclopentyl, or cyclohexyl. In further embodiments, R² is cyclopropyl.

In certain embodiments, the disclosure provides a compound having the structure of Formula II:

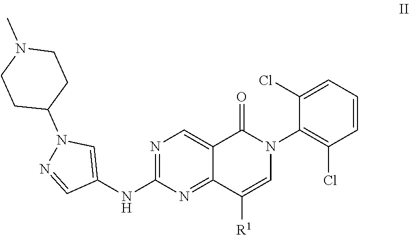

or a pharmaceutically acceptable salt thereof. In this structure, R¹ is halo, C₁₋₆alkyl, C₃₋₈cycloalkyl or C₂₋₆alkenyl; and R² is H, C₁₋₆alkyl, or C₃₋₈cycloalkyl. In some embodiments, R¹ is halo. In further embodiments, R¹ is chloro, bromo, fluoro, or iodo. In other embodiments, R¹ is chloro or bromo. In yet further embodiments, R¹ is C₁₋₄alkyl. In still other embodiments, R¹ is methyl, ethyl, n-propyl or i-propyl. In further embodiments, R¹ is methyl. In other embodiments, R¹ is C₃₋₇cycloalkyl. In still further embodiments, R¹ is cyclopropyl. In yet other embodiments, R¹ is C₁₋₄alkenyl. In further embodiments, R¹ is vinyl or isopropenyl. In other embodiments, R¹ is vinyl. In still further embodiments, R¹ is isopropenyl.

In certain embodiments, the disclosure provides a compound with the structure of formula IIa:

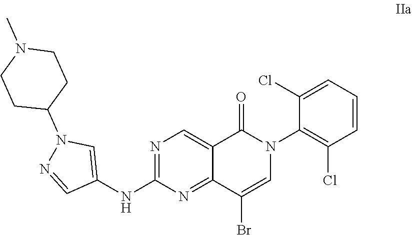 [SMILES: CN1CCC(n2cc(Nc3ncc4c(=O)n(-c5c(Cl)cccc5Cl)cc(Br)c4n3)cn2)CC1]

or a pharmaceutically acceptable salt thereof. In this structure, R¹ is halo, C₁₋₆alkyl, C₃-8cycloalkyl or C₂₋₆alkenyl; and R² is H, C₁₋₆alkyl, or C₃₋₈cycloalkyl. In some embodiments, R¹ is halo. In further embodiments, R¹ is chloro, bromo, fluoro, or iodo. In other embodiments, R¹ is chloro or bromo. In yet further embodiments, R¹ is C₁₋₄alkyl. In still other embodiments, R¹ is methyl, ethyl, n-propyl or i-propyl. In further embodiments, R¹ is methyl. In other embodiments, R¹ is C₃₋₇cycloalkyl. In still further embodiments, R¹ is cyclopropyl. In yet other embodiments, R¹ is C₁₋₄alkenyl. In further embodiments, R¹ is vinyl or isopropenyl. In other embodiments, R¹ is vinyl. In still further embodiments, R¹ is isopropenyl.

In certain embodiments, the disclosure provides a compound having the structure of Formula III:

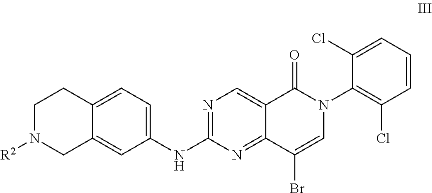

or a pharmaceutically acceptable salt thereof, wherein R² is H, C₁₋₆alkyl, or C₃₋₈cycloalkyl. In certain embodiments, R² is H. In certain embodiments, R² is C₁₋₆alkyl, or a pharmaceutically acceptable salt thereof. In certain embodiments, R² is methyl.

In certain embodiments, the disclosure provides a compound having the structure of Formula IV:

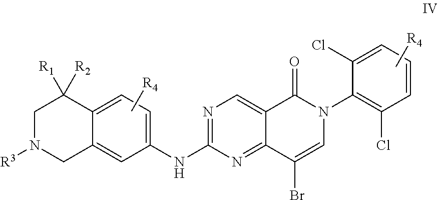

or a pharmaceutically acceptable salt thereof, wherein R1, R2=H; Me; Et; Pr; optionally wherein R1 and R2 are connected to form a 3-6 membered ring; R3=C1-4 small alkyl; C3-6 cycloalkyl ring; R4=H; C1-3 alkyl; CF3; OMe; OCF3; OCF2H; CN; halo.

In certain embodiments, the disclosure provides compound of Formula IVa:

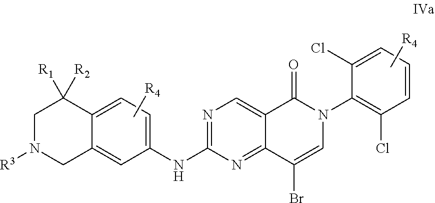

or a pharmaceutically acceptable salt thereof. In this formula, R¹ and R² are, independently, H, methyl, ethyl, or propyl; or R¹ and R² are connected together to form a 3-6 membered ring; R³ is C₁₋₄alkyl or C₃₋₆cycloalkyl; and R⁴ is H, C₁₋₃alkyl, CF₃, methoxy, OCF₃, OCF₂H, CN, or halo. In some embodiments, R¹ and/or R² are H. In other embodiments, R¹ and/or R² is methyl. In further embodiments, R¹ and/or R² are ethyl. In still other embodiments, R¹ and/or R² are propyl. In yet further embodiments, R¹ and R² are joined together to form a 3-6 membered ring. In other embodiments, R¹ and R² are joined together to form a cyclopropyl group. In further embodiments, R³ is C₁₋₄alkyl, such as methyl, ethyl, propyl, or butyl In yet other embodiments, R³ is C₃₋₆cycloalkyl, such as cyclopropyl. In still further embodiments, R⁴ is H. In other embodiments, R⁴ is C₁₋₃alkyl, such as methyl. In further embodiments, R⁴ is CF₃. In yet other embodiments, R⁴ is methoxy. In still further embodiments, R⁴ is OCF₃. In other embodiments, R⁴ is OCF₂H. In further embodiments, R⁴ is CN. In yet other embodiments, R⁴ is halo, such as fluoro, chloro, or bromo.

In certain embodiments, the disclosure provides a compound having the structure of Formula V:

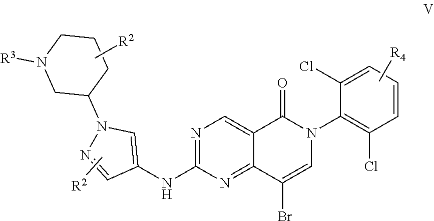

or a pharmaceutically acceptable salt thereof, wherein R2=H; Me; Et; Pr; R3=C1-4 small alkyl; C3-6 cycloalkyl ring; R4=H; C1-3 alkyl; CF3; OMe; OCF3; OCF2H; CN; halo.

In certain embodiments, the disclosure provides compounds of Formula Va:

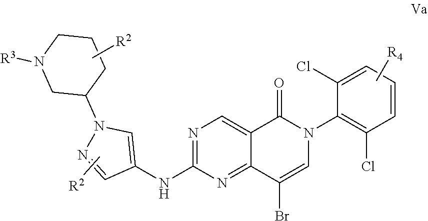

or a pharmaceutically acceptable salt thereof, wherein R² is H, methyl, ethyl, or propyl; R³ is C₁-4alkyl or C₃₋₆cycloalkyl; and R⁴ is H, C₁₋₃alkyl, CF₃, methoxy, OCF₃, OCF₂H, CN, or halo. In some embodiments, R² is H. In other embodiments, R² is methyl. In further embodiments, R² is ethyl. In still other embodiments, R² is propyl. In further embodiments, R³ is C₁₋₄alkyl, such as methyl, ethyl, propyl, or butyl In yet other embodiments, R³ is C₃₋₆cycloalkyl, such as cyclopropyl. In still further embodiments, R⁴ is H. In other embodiments, R⁴ is C₁₋₃alkyl, such as methyl. In further embodiments, R⁴ is CF₃. In yet other embodiments, R⁴ is methoxy. In still further embodiments, R⁴ is OCF₃. In other embodiments, R⁴ is OCF₂H. In further embodiments, R⁴ is CN. In yet other embodiments, R⁴ is halo, such as fluoro, chloro, or bromo.

In certain embodiments, the disclosure provides a compound having the structure of Formula VI:

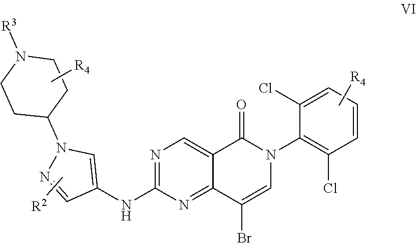

or a pharmaceutically acceptable salt thereof, wherein R2=H; Me; Et; Pr; R3=C1-4 small alkyl; C3-6 cycloalkyl ring; R4=H; C1-3 alkyl; CF3; OMe; OCF3; OCF2H; CN; halo.

In other embodiments, the disclosure provides compounds of Formula VIb:

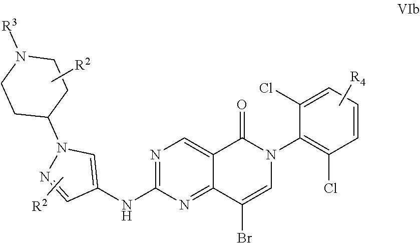

or a pharmaceutically acceptable salt thereof, wherein R² is H, methyl, ethyl, or propyl; R³ is C₁₋₄alkyl or C₃₋₆cycloalkyl; and R⁴ is H, C₁₋₃alkyl, CF₃, methoxy, OCF₃, OCF₂H, CN, or halo. In some embodiments, R² is H. In other embodiments, R² is methyl. In further embodiments, R² is ethyl. In still other embodiments, R² is propyl. In further embodiments, R³ is C₁₋₄alkyl, such as methyl, ethyl, propyl, or butyl In yet other embodiments, R³ is C₃₋₆cycloalkyl, such as cyclopropyl. In still further embodiments, R⁴ is H. In other embodiments, R⁴ is C₁₋₃alkyl, such as methyl. In further embodiments, R⁴ is CF₃. In yet other embodiments, R⁴ is methoxy. In still further embodiments, R⁴ is OCF₃. In other embodiments, R⁴ is OCF₂H. In further embodiments, R⁴ is CN. In yet other embodiments, R⁴ is halo, such as fluoro, chloro, or bromo.

In further embodiments, the disclosure provides compounds of formula VIa:

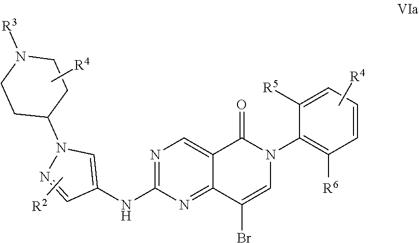

or a pharmaceutically acceptable salt thereof, wherein R² is H, methyl, ethyl, or propyl; R³ is C₁-4alkyl or C₃₋₆cycloalkyl; R⁴ is H, C₁₋₃alkyl, CF₃, methoxy, OCF₃, OCF₂H, CN, or halo; and R⁵ and R⁶ are, independently, is H, halo, or C₁₋₆alkyl. In some embodiments, R² is H. In other embodiments, R² is methyl. In further embodiments, R² is ethyl. In still other embodiments, R² is propyl. In further embodiments, R³ is C₁₋₄alkyl, such as methyl, ethyl, propyl, or butyl. In yet other embodiments, R³ is C₃₋₆cycloalkyl, such as cyclopropyl. In still further embodiments, R⁴ is H. In other embodiments, R⁴ is C₁₋₃alkyl, such as methyl. In further embodiments, R⁴ is CF₃. In yet other embodiments, R⁴ is methoxy. In still further embodiments, R⁴ is OCF₃. In other embodiments, R⁴ is OCF₂H. In further embodiments, R⁴ is CN. In yet other embodiments, R⁴ is halo, such as fluoro, chloro, or bromo. In yet further embodiments, R⁵ and/or R⁶ are H. In other embodiments, R⁵ and/or R⁶ are halo, such as fluoro, chloro, or bromo, or such as fluoro. In further embodiments, R⁵ and/or R⁶ are C₁₋₆alkyl, such as methyl, ethyl, propyl, or butyl, or such as methyl.

In other embodiments, the disclosure provides compounds of Formula VII:

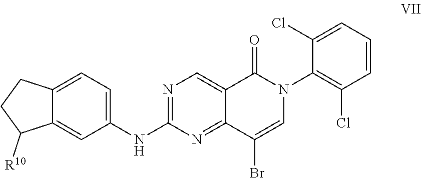

or a pharmaceutically acceptable salt, wherein R¹⁰ is H, OH, NH₂, NH(C₁₋₆alkyl), or N(C₁₋₆alkyl)(C₁₋₆alkyl). In some embodiments, R¹⁰ is H. In other embodiments, R¹⁰ is OH. In further embodiments, R¹⁰ is NH₂. In yet other embodiments, R¹⁰ is NH(C₁₋₆alkyl). In still further embodiments, R¹⁰ is N(C₁₋₆alkyl)(C₁₋₆alkyl), such as N(CH3)₂.

Further embodiments of the disclosure provide the compounds as shown in Table 1, or pharmaceutically acceptable salts thereof:

TABLE 1
Exemplary compounds of the invention

| | |
|---|---|
| 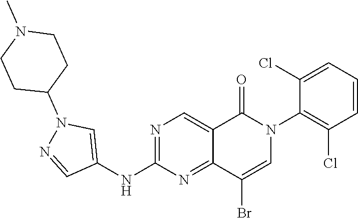 [SMILES: CN1CCC(n2cc(Nc3ncc4c(=O)n(-c5c(Cl)cccc5Cl)cc(Br)c4n3)cn2)CC1] | P1 |
| 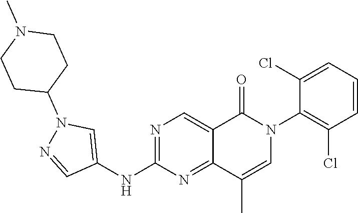 [SMILES: Cc1cn(-c2c(Cl)cccc2Cl)c(=O)c2cnc(Nc3cnn(C4CCN(C)CC4)c3)nc12] | P2 |
| 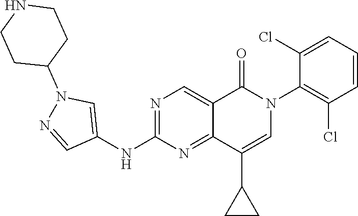 [SMILES: O=c1c2cnc(Nc3cnn(C4CCNCC4)c3)nc2c(C2CC2)cn1-c1c(Cl)cccc1Cl] | P3 |
| 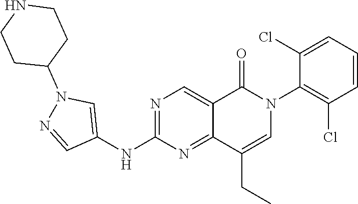 [SMILES: CCc1cn(-c2c(Cl)cccc2Cl)c(=O)c2cnc(Nc3cnn(C4CCNCC4)c3)nc12] | P4 |
| 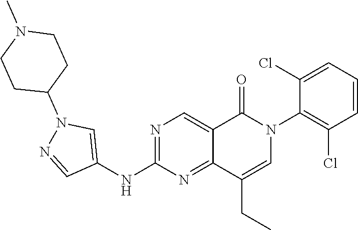 [SMILES: CCc1cn(-c2c(Cl)cccc2Cl)c(=O)c2cnc(Nc3cnn(C4CCN(C)CC4)c3)nc12] | P5 |
| 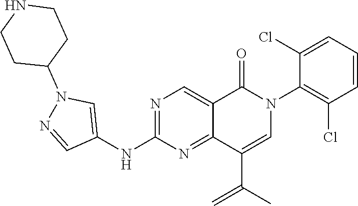 [SMILES: C=C(C)c1cn(-c2c(Cl)cccc2Cl)c(=O)c2cnc(Nc3cnn(C4CCNCC4)c3)nc12] | P6 |
| 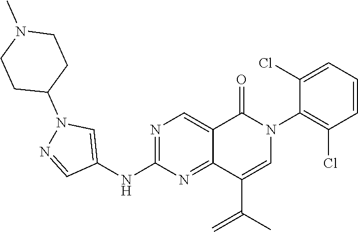 [SMILES: C=C(C)c1cn(-c2c(Cl)cccc2Cl)c(=O)c2cnc(Nc3cnn(C4CCN(C)CC4)c3)nc12] | P7 |
| 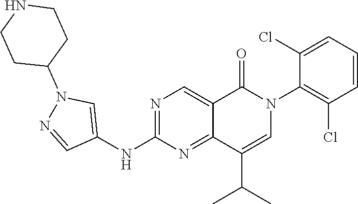 [SMILES: CC(C)c1cn(-c2c(Cl)cccc2Cl)c(=O)c2cnc(Nc3cnn(C4CCNCC4)c3)nc12] | P8 |
| 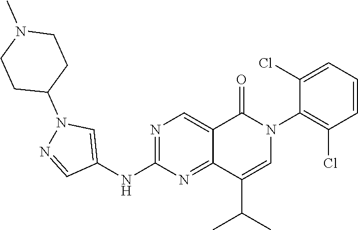 [SMILES: CC(C)c1cn(-c2c(Cl)cccc2Cl)c(=O)c2cnc(Nc3cnn(C4CCN(C)CC4)c3)nc12] | P9 |
| 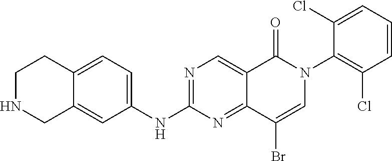 [SMILES: O=c1c2cnc(Nc3ccc4c(c3)CNCC4)nc2c(Br)cn1-c1c(Cl)cccc1Cl] | P10 |
| 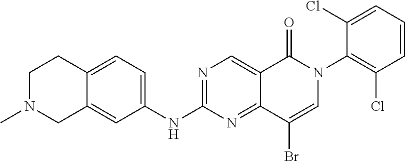 [SMILES: CN1CCc2ccc(Nc3ncc4c(=O)n(-c5c(Cl)cccc5Cl)cc(Br)c4n3)cc2C1] | P11 |
| 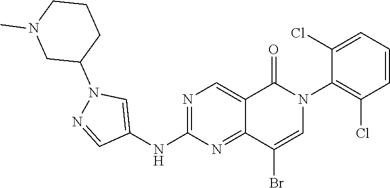 [SMILES: CN1CCCC(n2cc(Nc3ncc4c(=O)n(-c5c(Cl)cccc5Cl)cc(Br)c4n3)cn2)C1] | P12 |
| 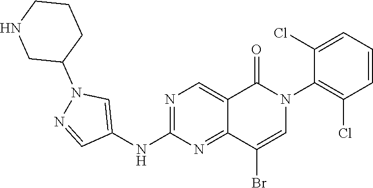 [SMILES: O=c1c2cnc(Nc3cnn(C4CCCNC4)c3)nc2c(Br)cn1-c1c(Cl)cccc1Cl] | P13 |
| 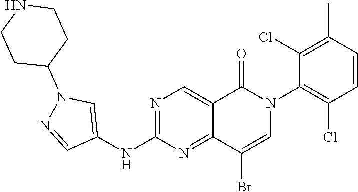 [SMILES: Cc1ccc(Cl)c(-n2cc(Br)c3nc(Nc4cnn(C5CCNCC5)c4)ncc3c2=O)c1Cl] | P14 |
| 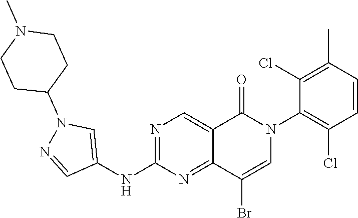 [SMILES: Cc1ccc(Cl)c(-n2cc(Br)c3nc(Nc4cnn(C5CCN(C)CC5)c4)ncc3c2=O)c1Cl] | P15 |
| 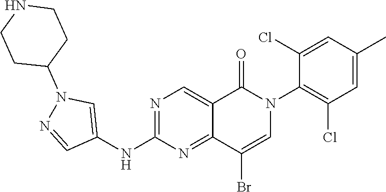 [SMILES: Cc1cc(Cl)c(-n2cc(Br)c3nc(Nc4cnn(C5CCNCC5)c4)ncc3c2=O)c(Cl)c1] | P16 |
| 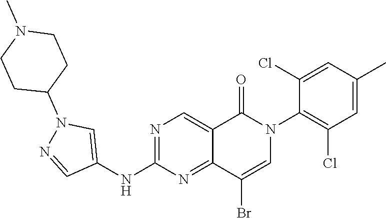 [SMILES: Cc1cc(Cl)c(-n2cc(Br)c3nc(Nc4cnn(C5CCN(C)CC5)c4)ncc3c2=O)c(Cl)c1] | P17 |
| 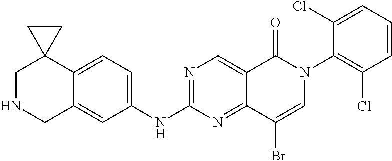 [SMILES: O=c1c2cnc(Nc3ccc4c(c3)CNCC43CC3)nc2c(Br)cn1-c1c(Cl)cccc1Cl] | P19 |
| 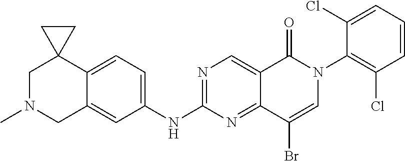 [SMILES: CN1Cc2cc(Nc3ncc4c(=O)n(-c5c(Cl)cccc5Cl)cc(Br)c4n3)ccc2C2(CC2)C1] | P20 |
| 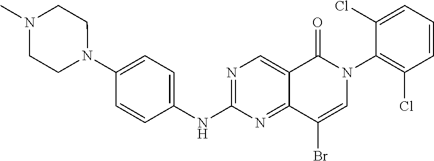 [SMILES: CN1CCN(c2ccc(Nc3ncc4c(=O)n(-c5c(Cl)cccc5Cl)cc(Br)c4n3)cc2)CC1] | P21 |
| 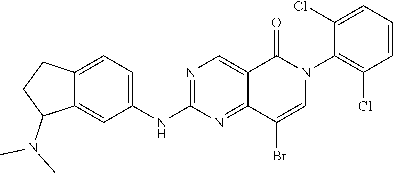 [SMILES: CN(C)C1CCc2ccc(Nc3ncc4c(=O)n(-c5c(Cl)cccc5Cl)cc(Br)c4n3)cc21] | P22 |
| 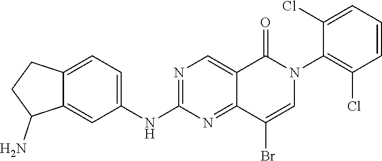 [SMILES: NC1CCc2ccc(Nc3ncc4c(=O)n(-c5c(Cl)cccc5Cl)cc(Br)c4n3)cc21] | P23 |
| 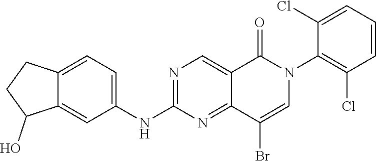 [SMILES: O=c1c2cnc(Nc3ccc4c(c3)C(O)CC4)nc2c(Br)cn1-c1c(Cl)cccc1Cl] | P24 |
| 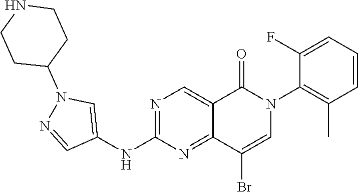 [SMILES: Cc1cccc(F)c1-n1cc(Br)c2nc(Nc3cnn(C4CCNCC4)c3)ncc2c1=O] | P25 |
| 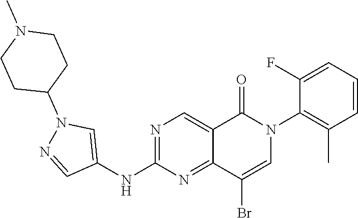 [SMILES: Cc1cccc(F)c1-n1cc(Br)c2nc(Nc3cnn(C4CCN(C)CC4)c3)ncc2c1=O] | P26 |
| 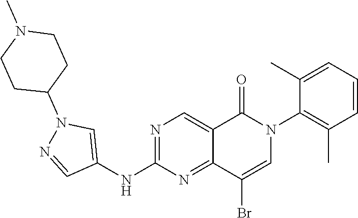 [SMILES: Cc1cccc(C)c1-n1cc(Br)c2nc(Nc3cnn(C4CCN(C)CC4)c3)ncc2c1=O] | P27 |

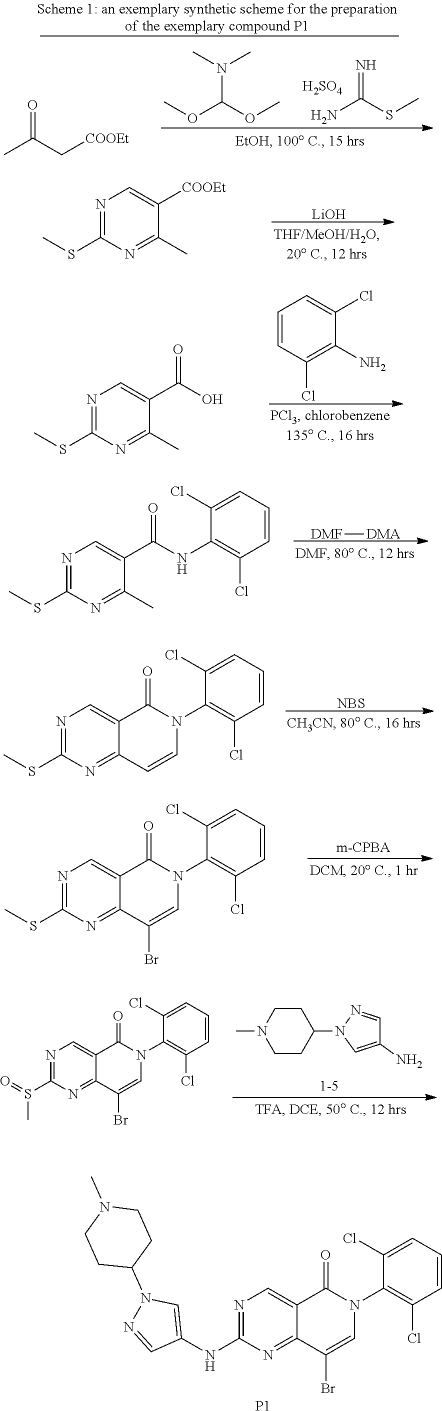

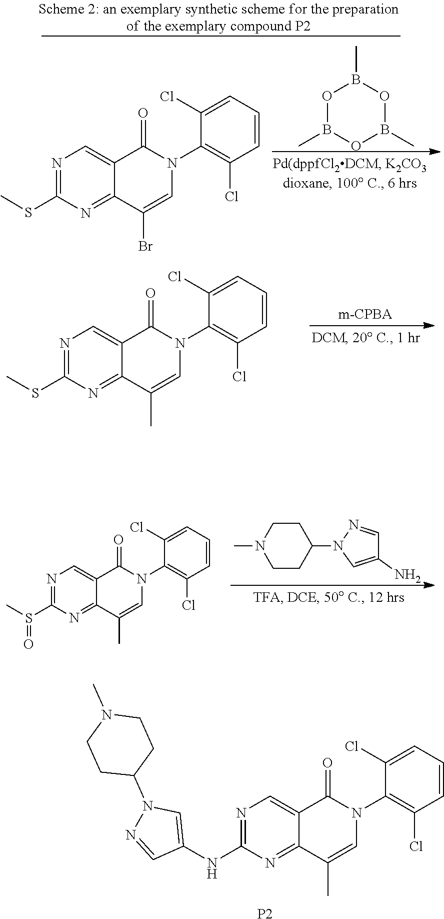

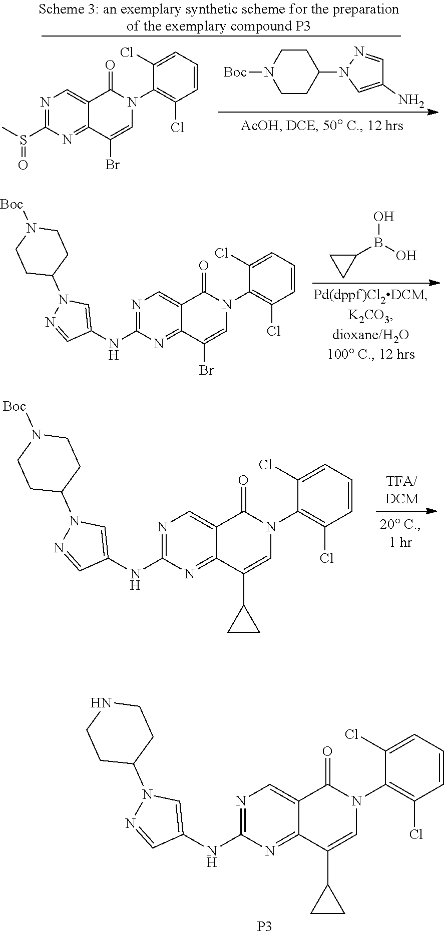

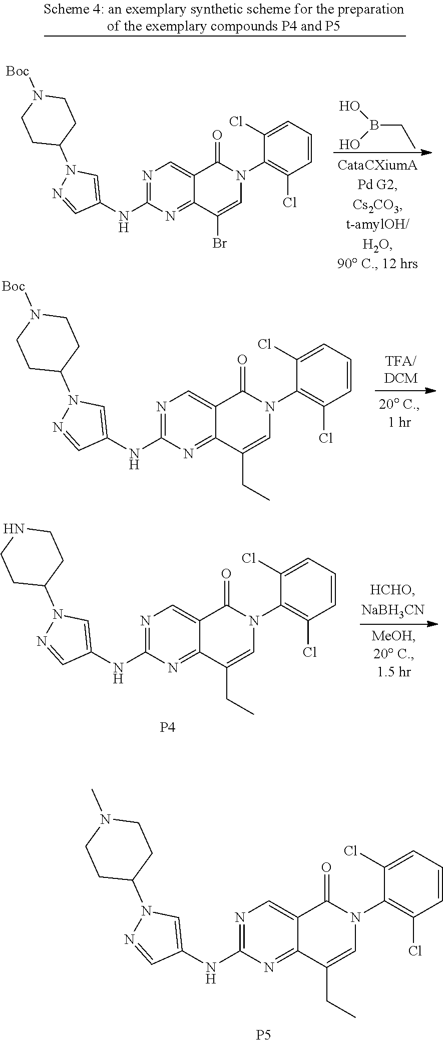

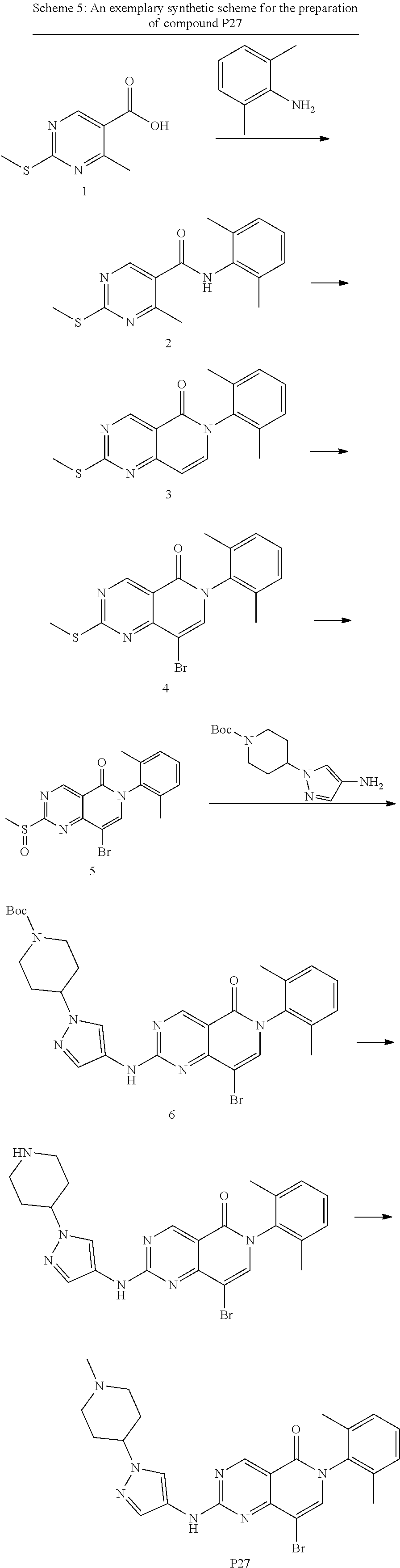

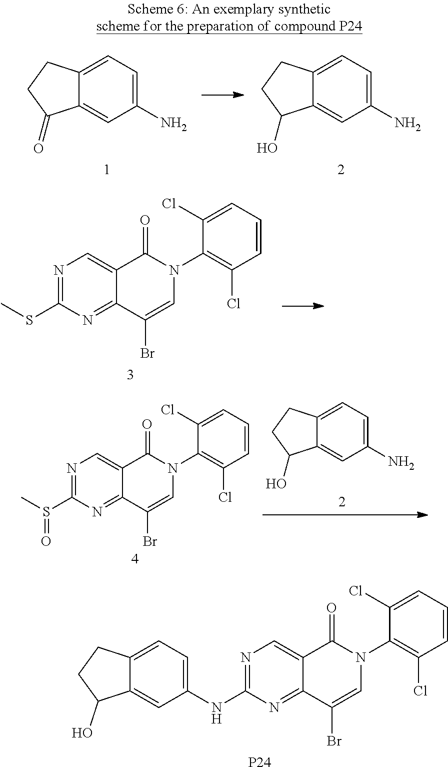

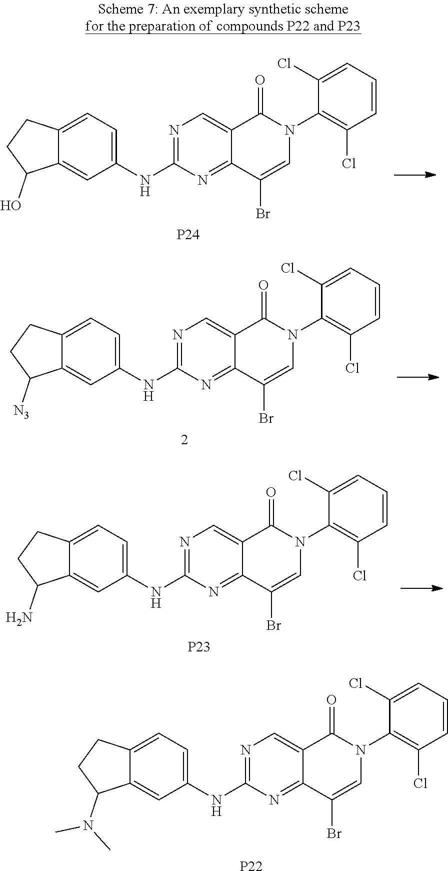

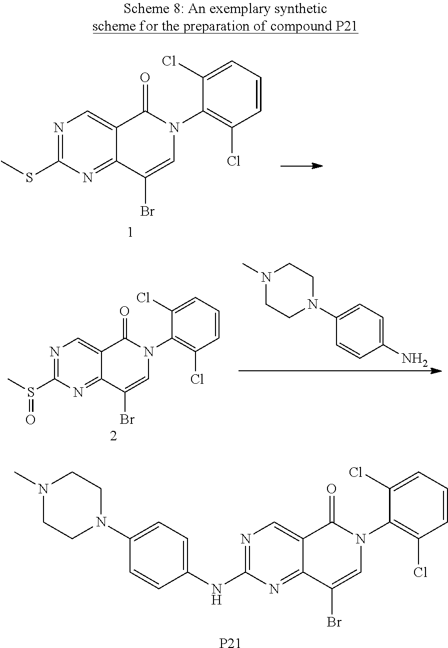

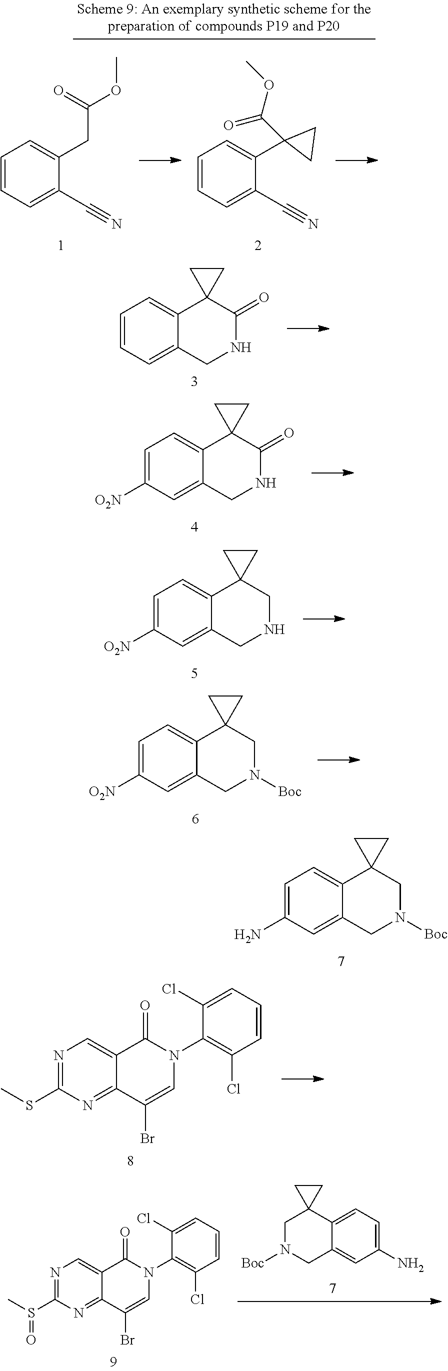

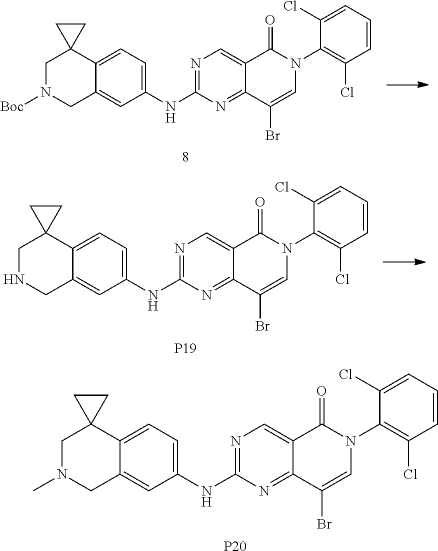

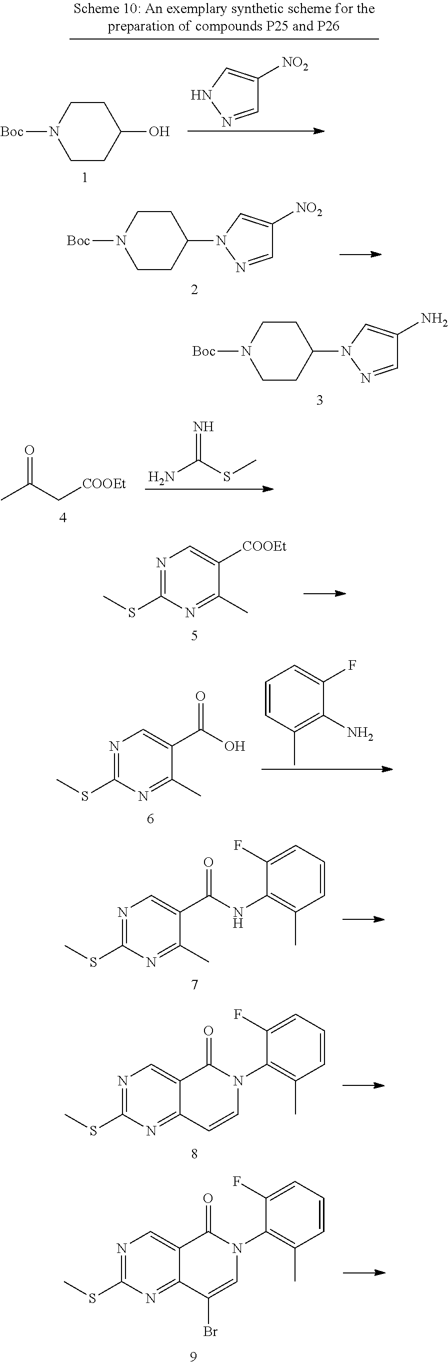

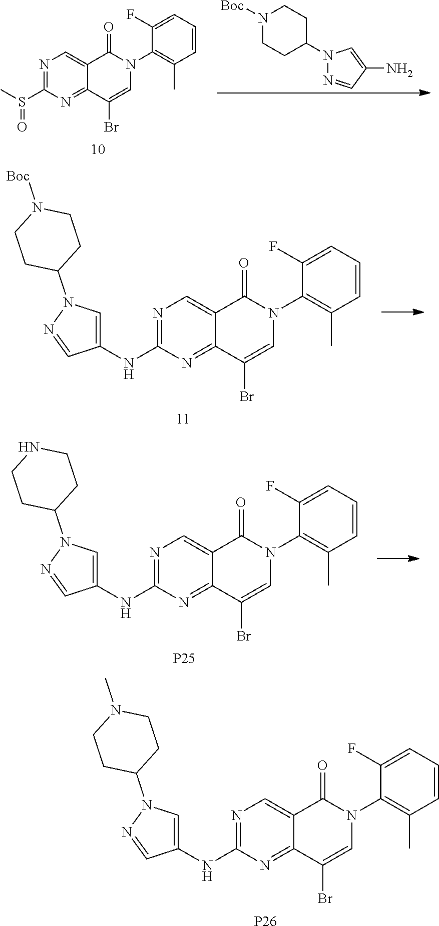

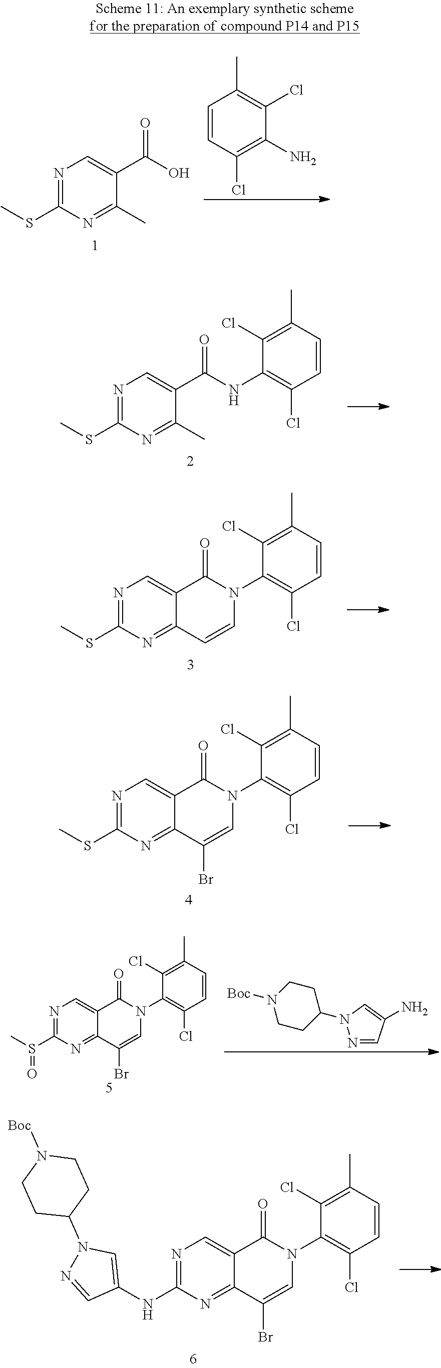

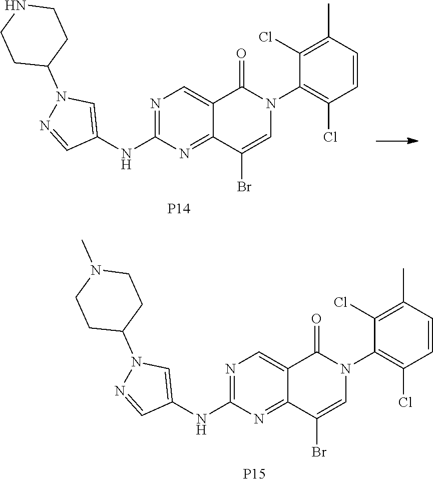

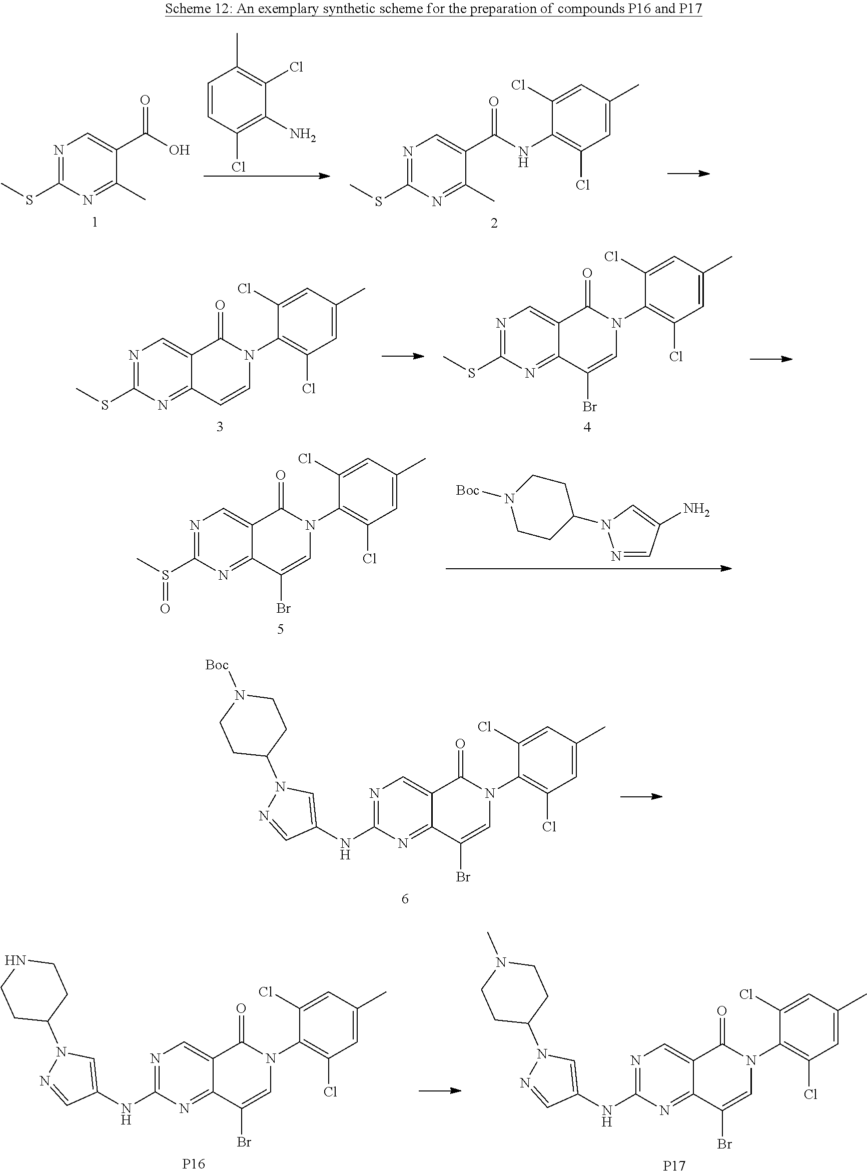

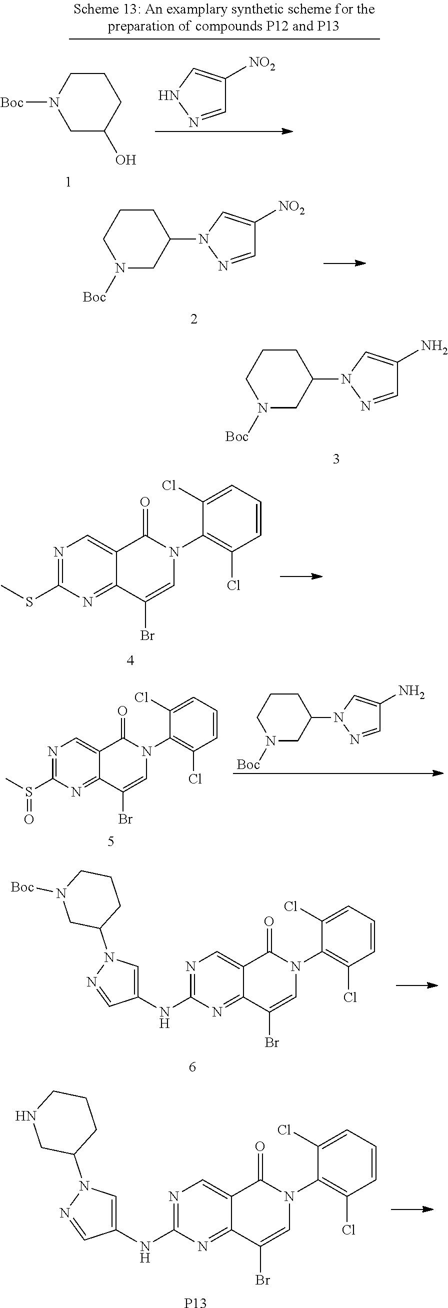

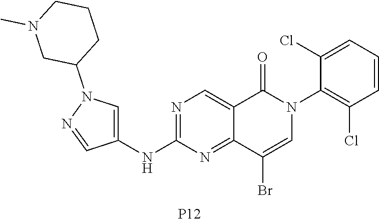

In exemplary embodiments, formulations as disclosed herein may comprise active agent as disclosed herein at a concentration of about 100, about 2%, about 300, about 400 about 5%, about 6%, about 7%, about 8%, about 9%, about 10%, about 110%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%. In exemplary embodiments, formulations as disclosed herein may comprise active agent at a concentration of about 1 to about 20%, of about 5% to about 25%, about 10% to about 20%, or about 15% to about 18%, about 30% to about 70%, about 35% to about 65%, about 63.13%, and about 40% to about 64% w/w.

In an exemplary formulations as disclosed herein, active agent as disclosed herein will represent approximately 1 wt % to 75 wt %, preferably 2 wt % to 30 wt %, more preferably 5 wt. % to 20 wt. % of the total weight of the formulation.

In other embodiments, the pharmaceutical compositions as disclosed herein further comprise one or more additional materials such as a pharmaceutically compatible carrier, binder, viscosity modifier, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

### Pharmaceutical Dosage Forms

The compounds and compositions as disclosed herein can be provided in the form of a pharmaceutical composition, such as a pharmaceutical dosage form, such as a minicapsule, a capsule, a tablet, an implant, a troche, a lozenge (minitablet), a temporary or permanent suspension, an ovule, a suppository, a wafer, a chewable tablet, a quick or fast dissolving tablet, an effervescent tablet, a granule, a film, a sprinkle, a pellet, a bead, a pill, a powder, a triturate, a platelet, a strip or a sachet. Compositions can also be administered after being mixed with, for example yoghurt or fruit juice and swallowed or followed with a drink or beverage. These forms are well known in the art and are packaged appropriately. The compositions can be formulated for oral or rectal delivery.

Tablets prepared for oral administration according to the invention, and manufactured using direct compression, will generally contain other inactive additives such as binders, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are for example, magnesium stearate, calcium stearate, stearic acid, and hydrogenated vegetable oil (preferably comprised of hydrogenated and refined triglycerides of stearic and palmitic acids at about 1 wt. % to 5 wt. %, most preferably less than about 2 wt. %). Lubricants may be present in a concentration of, for example, from about 0.25 wt. % to about 3 wt. %, 0.5 wt. % to about 2.0 wt. %, from about 0.75% to about 1.5%.

Disintegrants are used to facilitate disintegration of the tablet, thereby increasing the erosion rate relative to the dissolution rate, and are generally starches, clays, celluloses, algins, gums, or crosslinked polymers (e.g., crosslinked polyvinyl pyrrolidone). Fillers include, for example, materials such as silicon dioxide, titanium dioxide, alumina, talc, kaolin, powdered cellulose, and microcrystalline cellulose, as well as soluble materials such as mannitol, urea, sucrose, lactose, lactose monohydrate, dextrose, sodium chloride, and sorbitol. Solubility-enhancers, including solubilizers per se, emulsifiers, and complexing agents (e.g., cyclodextrins), may also be advantageously included in the present formulations. Stabilizers, as well known in the art, are used to inhibit or retard drug decomposition reactions that include, by way of example, oxidative reactions. Disintegrants may be present in a concentration of, for example, from about 0.25 wt. % to about 3 wt. %, 0.5 wt. % to about 2.0 wt. %, from about 0.75% to about 1.5%.

Shellac, also called purified lac, a refined product obtained from the, resinous secretion of an insect. This coating dissolves in media of pH>7.

Colorants, detackifiers, surfactants, antifoaming agents, lubricants, stabilizers such as hydroxy propyl cellulose, acid/base may be added to the coatings besides plasticizers to solubilize or disperse the coating material, and to improve coating performance and the coated product.

In carrying out the method as disclosed herein, the compositions of the disclosure may be administered to mammalian species, such as dogs, cats, humans, etc. and as such may be incorporated in a conventional systemic dosage form, such as a tablet, capsule, or elixir. The above dosage forms will also include the necessary carrier material, excipient, viscosity modifier, lubricant, buffer, antibacterial, bulking agent (such as mannitol), anti-oxidants (ascorbic acid of sodium bisulfate) or the like.

The dose administered may be carefully adjusted according to age, weight and condition of the patient, as well as the route of administration, dosage form and regimen and the desired result.

The compositions of the disclosure may be administered in the dosage forms in single or divided doses of one to four times daily, or may be administered multiple times per day. It may be advisable to start a patient on a low dose combination and work up gradually to a high dose combination.

Tablets of various sizes can be prepared, e.g., of about 2 to 2000 mg in total weight, containing one or more active ingredients, with the remainder being a physiologically acceptable carrier of other materials according to accepted practice. Gelatin capsules can be similarly formulated.

Liquid formulations can also be prepared by dissolving or suspending one or the combination of active substances in a conventional liquid vehicle acceptable for administration so as to provide the desired dosage in, for example, one to four teaspoonfuls.

Dosage forms can be administered to the patient on a regimen of, for example, one, two, three, four, five, six, or other multiple doses per day.

In order to more finely regulate the dosage schedule, the active agents may be administered separately in individual dosage units at the same time or carefully coordinated times. The respective substances can be individually formulated in separate unit dosage forms in a manner similar to that described herein.

In formulating the compositions, the active agents, in the amounts described herein, may be compounded according to accepted practice with a physiologically acceptable vehicle, carrier, excipient, binder, viscosity modifier, preservative, stabilizer, flavor, etc., in the particular type of unit dosage form.

When formulated as a capsule, the capsule can be a hard or soft gelatin capsule, a starch capsule, a cellulosic capsule, or a multi particulate system. Multi particulate systems consist of a dosage form based on a plurality of drug loaded spheres, which may be prepared by layering drug onto a core, usually a sugar-starch mixture sphere of around 0.8 mm diameter, until a sufficient level is reached, and then providing a drug release barrier around the drug-loaded sphere. Drug-loaded spheres can also be made by wet massing a mixture of drug and excipients, forcing the wet mass through a perforated screen to form short strands which are rounded in a spheronization apparatus before drying and having the drug release barrier applied. The drug release barrier can be a wax, such as carnauba wax or glyceryl fatty acid esters, or a polymeric barrier, such as a mixture of ethyl cellulose and hydroxypropylmethylcellulose. These work well for moderately soluble drugs with doses in the units of milligrams to less than a few hundred milligrams per day. Multi particulate systems are usually filled into capsules to provide unit dose forms because of the damage caused to such particles in trying to compress them into tablets. Total dose contained in a single unit is constrained by the loading possible in a hard gelatin capsule of easily swallowable size and is usually not more than a few hundred milligrams. Although not limited to capsules, such dosage forms can further be coated with, for example, a seal coating, an enteric coating, an extended release coating, or a targeted delayed release coating. These various coatings are known in the art, but for clarity, the following brief descriptions are provided: seal coating, or coating with isolation layers: Thin layers of up to 20 microns in thickness can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are preferred for this application. HPMC and ethyl cellulose in combination, or EUDRAGIT® E100, may be particularly suitable for taste masking applications. Traditional enteric coating materials listed elsewhere can also be applied to form an isolating layer.

Oral dosage forms of the controlled release active agent formulation of the disclosure can be in the form of a multiparticulate formulation or a tablet. The term “multiparticulate” as used herein includes discrete particles, pellets, mini-tablets and mixtures or combinations thereof. A multiparticulate oral dosage form according to the disclosure can comprise a blend of two or more populations of particles, pellets or mini-tablets having different in vitro and/or in vivo release characteristics. For example, the multiparticulate oral dosage form can comprise a blend of an instant release component and a controlled release component contained in a suitable capsule, for example hard or soft gelatin capsules. If the multiparticulate formulation is filled into a capsule it may be administered by swallowing the capsule or by opening said capsule and sprinkling the contents onto food. Alternatively the multiparticulate may be presented in a sachet.

The particles and one or more auxiliary excipient materials can be compressed into tablet form such as a multilayer tablet. Typically a multilayer tablet may comprise two layers which may contain the same or different levels of the same active ingredient having the same or different release characteristics or may contain a different active ingredient in each layer. Such a multilayer tablet may optionally be coated with a controlled release polymer so as to provide additional controlled release properties.

As indicated above the active agent formulations and oral dosage forms of the present disclosure may comprise auxiliary excipients such as for example diluents, lubricants, surfactants, disintegrants, plasticizers, anti-tack agents, opacifying agents, pigments, flavorings and such like. As will be appreciated by those skilled in the art, the exact choice of excipients and their relative amounts will depend to some extent on the final oral dosage form into which the controlled release active agent formulation is incorporated.

Suitable diluents include for example pharmaceutically acceptable inert fillers such as microcrystalline cellulose, lactose, dibasic calcium phosphate, saccharides, and/or mixtures of any of the foregoing. Examples of diluents include microcrystalline celluloses such as those sold under the Trade Mark Avicel; including for example Avicel pH101, Avicel pH102, Avicel pH112, Avicel pH200, Avicel pH301 and Avicel pH302; lactose such as lactose monohydrate, lactose anhydrous and Pharmatose DCL21 (Pharmatose is a Trade Mark), including anhydrous, monohydrate and spray dried forms; dibasic calcium phosphate such as Emcompress (Emcompress is a Trade Mark); mannitol; starch; sorbitol; sucrose; and glucose. Suitable lubricants, including agents that act on the flowability of the powder to be compressed are, for example, colloidal silicon dioxide such as Aerosil 200; talc; stearic acid, magnesium stearate, calcium stearate and sodium stearyl fumarate. Suitable disintegrants include for example lightly crosslinked polyvinyl pyrrolidone, corn starch, potato starch, maize starch and modified starches, croscarmellose sodium, cross-povidone, sodium starch glycolate and combinations and mixtures thereof.

According to a further aspect of the disclosure there is provided a controlled release active agent formulation for oral administration comprising a blend of particles. According to a still further aspect of the disclosure there is provided a controlled release active agent formulation for oral administration comprising a blend of particles as hereinbefore defined in admixture with an immediate release form of active agent or a pharmaceutically acceptable salt thereof to ensure a rapid attainment of effective therapeutic blood levels. Preferably, the immediate release form of active agent comprises pellets as hereinbefore defined without said rate-controlling membrane.

Extended or delayed release coatings are designed to effect delivery over an extended period of time. The extended or delayed release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended or delayed release dosage forms can be readily designed by one skilled in art to achieve delivery to both the small and large intestines, to only the small intestine, or to only the large intestine, depending upon the choice of coating materials and/or coating thickness.

Enteric coatings are mixtures of acceptable excipients which are applied to, combined with, mixed with or otherwise added to the carrier or composition. The coating may be applied to a compressed or molded or extruded tablet, a gelatin capsule, and/or pellets, beads, granules or particles of the carrier or composition. The coating may be applied through an aqueous dispersion or after dissolving in appropriate solvent.

Dosage forms of the compositions as disclosed herein can also be formulated as enteric coated delayed release oral dosage forms, i.e., as an oral dosage form of a composition as described herein which utilizes an enteric coating to affect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

Delayed release coating compositions comprise a polymeric material, e.g., cellulose butyrate phthalate, cellulose hydrogen phthalate, cellulose propionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof. Preferred enteric coatings herein are comprised of methacrylic acid copolymers, types A, B, or C, which are commercially available from Rohm Tech, Inc. (Malden, Mass.), and water-based dispersions of cellulose acetate phthalate latex, which is commercially available from Eastman Fine Chemicals (Kingsport, Tenn.).

### Neoplasia

The disclosure provides a method for treating and/or preventing neoplasia in a patient in need of such treatment or prevention, comprising: administering to the patient therapeutically effective amounts of the compound(s) or composition(s) as disclosed herein, combinations thereof, and pharmaceutically acceptable salts thereof, optionally in combination with, for example, a neoplasia treating agent. The term “neoplasia” as used herein refers also to tumors, proliferative diseases, malignancies and their metastases. Examples for cancer diseases include, for example, carcinoma, a sarcoma, a lymphoma or leukemia, a germ cell tumor, a blastoma, or other cancers. Carcinomas include without limitation epithelial neoplasms, squamous cell neoplasms squamous cell carcinoma, basal cell neoplasms basal cell carcinoma, transitional cell papillomas and carcinomas, adenomas and adenocarcinomas (glands), adenoma, adenocarcinoma, linitis plastica insulinoma, glucagonoma, gastrinoma, vipoma, cholangiocarcinoma, hepatocellular carcinoma, adenoid cystic carcinoma, carcinoid tumor of appendix, prolactinoma, oncocytoma, hurthle cell adenoma, renal cell carcinoma, grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, warthin tumor, thymoma, specialized gonadal neoplasms, sex cord stromal tumor, thecoma, granulosa cell tumor, arrhenoblastoma, sertoli leydig cell tumor, glomus tumors, paraganglioma, pheochromocytoma, glomus tumor, nevi and melanomas, melanocytic nevus, malignant melanoma, melanoma, nodular melanoma, dysplastic nevus, lentigo maligna melanoma, superficial spreading melanoma, and malignant acral lentiginous melanoma. Sarcoma includes without limitation Askin's tumor, botryodies, chondrosarcoma, Ewing's sarcoma, malignant hemangio endothelioma, malignant schwannoma, osteosarcoma, soft tissue sarcomas including: alveolar soft part sarcoma, angiosarcoma, cystosarcoma phyllodes, dermatofibrosarcoma, desmoid tumor, desmoplastic small round cell tumor, epithelioid sarcoma, extraskeletal chondrosarcoma, extraskeletal osteosarcoma, fibrosarcoma, hemangiopericytoma, hemangiosarcoma, kaposi's sarcoma, leiomyosarcoma, liposarcoma, lymphangiosarcoma, lymphosarcoma, malignant fibrous histiocytoma, neurofibrosarcoma, rhabdomyosarcoma, and synovialsarcoma. Lymphoma and leukemia include without limitation chronic lymphocytic leukemia/small lymphocytic lymphoma, B-cell prolymphocytic leukemia, lymphoplasmacytic lymphoma (such as waldenstrom macroglobulinemia), splenic marginal zone lymphoma, plasma cell myeloma, plasmacytoma, monoclonal immunoglobulin deposition diseases, heavy chain diseases, extranodal marginal zone B cell lymphoma, also called malt lymphoma, nodal marginal zone B cell lymphoma (nmzl), follicular lymphoma, mantle cell lymphoma, diffuse large B cell lymphoma, mediastinal (thymic) large B cell lymphoma, intravascular large B cell lymphoma, primary effusion lymphoma, burkitt lymphoma/leukemia, T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/sezary syndrome, primary cutaneous CD30-positive T cell lymphoproliferative disorders, primary cutaneous anaplastic large cell lymphoma, lymphomatoid papulosis, angioimmunoblastic T cell lymphoma, peripheral T cell lymphoma, unspecified, anaplastic large cell lymphoma, classical hodgkin lymphomas (nodular sclerosis, mixed cellularity, lymphocyte-rich, lymphocyte depleted or not depleted), and nodular lymphocyte-predominant hodgkin lymphoma. Germ cell tumors include without limitation germinoma, dysgerminoma, seminoma, nongerminomatous germ cell tumor, embryonal carcinoma, endodermal sinus tumor, choriocarcinoma, teratoma, polyembryoma, and gonadoblastoma. Blastoma includes without limitation nephroblastoma, medulloblastoma, and retinoblastoma. Other cancers include without limitation labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma.

In a further embodiment, the cancer may be a lung cancer including non-small cell lung cancer and small cell lung cancer (including small cell carcinoma (oat cell cancer), mixed small cell/large cell carcinoma, and combined small cell carcinoma), colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemia, lymphoma, myeloma, or a solid tumor.

In embodiments, the cancer comprises an acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom's macroglobulinemia; or Wilm's tumor. The methods of the invention can be used to characterize these and other cancers. Thus, characterizing a phenotype can be providing a diagnosis, prognosis or theranosis of one of the cancers disclosed herein.

In some embodiments, the cancer comprises an acute myeloid leukemia (AML), breast carcinoma, cholangiocarcinoma, colorectal adenocarcinoma, extrahepatic bile duct adenocarcinoma, female genital tract malignancy, gastric adenocarcinoma, gastroesophageal adenocarcinoma, gastrointestinal stromal tumors (GIST), glioblastoma, head and neck squamous carcinoma, leukemia, liver hepatocellular carcinoma, low grade glioma, lung bronchioloalveolar carcinoma (BAC), lung non-small cell lung cancer (NSCLC), lung small cell cancer (SCLC), lymphoma, male genital tract malignancy, malignant solitary fibrous tumor of the pleura (MSFT), melanoma, multiple myeloma, neuroendocrine tumor, nodal diffuse large B-cell lymphoma, non epithelial ovarian cancer (non-EOC), ovarian surface epithelial carcinoma, pancreatic adenocarcinoma, pituitary carcinomas, oligodendroglioma, prostatic adenocarcinoma, retroperitoneal or peritoneal carcinoma, retroperitoneal or peritoneal sarcoma, small intestinal malignancy, soft tissue tumor, thymic carcinoma, thyroid carcinoma, or uveal melanoma.

As described herein, the invention provides methods and compositions useful for analysis, detection, characterization, imaging, prevention, and treatment of various diseases and disorders. In various embodiments, the disease or disorder comprises a cancer, a premalignant condition, an inflammatory disease, an immune disease, an autoimmune disease or disorder, a cardiovascular disease or disorder, neurological disease or disorder, infectious disease or pain. The cancer can include without limitation one of acute lymphoblastic leukemia; acute myeloid leukemia; adrenocortical carcinoma; AIDS-related cancers; AIDS-related lymphoma; anal cancer; appendix cancer; astrocytomas; atypical teratoid/rhabdoid tumor; basal cell carcinoma; bladder cancer; brain stem glioma; brain tumor (including brain stem glioma, central nervous system atypical teratoid/rhabdoid tumor, central nervous system embryonal tumors, astrocytomas, craniopharyngioma, ependymoblastoma, ependymoma, medulloblastoma, medulloepithelioma, pineal parenchymal tumors of intermediate differentiation, supratentorial primitive neuroectodermal tumors and pineoblastoma); breast cancer; bronchial tumors; Burkitt lymphoma; cancer of unknown primary site; carcinoid tumor; carcinoma of unknown primary site; central nervous system atypical teratoid/rhabdoid tumor; central nervous system embryonal tumors; cervical cancer; childhood cancers; chordoma; chronic lymphocytic leukemia; chronic myelogenous leukemia; chronic myeloproliferative disorders; colon cancer; colorectal cancer; craniopharyngioma; cutaneous T-cell lymphoma; endocrine pancreas islet cell tumors; endometrial cancer; ependymoblastoma; ependymoma; esophageal cancer; esthesioneuroblastoma; Ewing sarcoma; extracranial germ cell tumor; extragonadal germ cell tumor; extrahepatic bile duct cancer; gallbladder cancer; gastric (stomach) cancer; gastrointestinal carcinoid tumor; gastrointestinal stromal cell tumor; gastrointestinal stromal tumor (GIST); gestational trophoblastic tumor; glioma; hairy cell leukemia; head and neck cancer; heart cancer; Hodgkin lymphoma; hypopharyngeal cancer; intraocular melanoma; islet cell tumors; Kaposi sarcoma; kidney cancer; Langerhans cell histiocytosis; laryngeal cancer; lip cancer; liver cancer; lung cancer; malignant fibrous histiocytoma bone cancer; medulloblastoma; medulloepithelioma; melanoma; Merkel cell carcinoma; Merkel cell skin carcinoma; mesothelioma; metastatic squamous neck cancer with occult primary; mouth cancer; multiple endocrine neoplasia syndromes; multiple myeloma; multiple myeloma/plasma cell neoplasm; mycosis fungoides; myelodysplastic syndromes; myeloproliferative neoplasms; nasal cavity cancer; nasopharyngeal cancer; neuroblastoma; Non-Hodgkin lymphoma; nonmelanoma skin cancer; non-small cell lung cancer; oral cancer; oral cavity cancer; oropharyngeal cancer; osteosarcoma; other brain and spinal cord tumors; ovarian cancer; ovarian epithelial cancer; ovarian germ cell tumor; ovarian low malignant potential tumor; pancreatic cancer; papillomatosis; paranasal sinus cancer; parathyroid cancer; pelvic cancer; penile cancer; pharyngeal cancer; pineal parenchymal tumors of intermediate differentiation; pineoblastoma; pituitary tumor; plasma cell neoplasm/multiple myeloma; pleuropulmonary blastoma; primary central nervous system (CNS) lymphoma; primary hepatocellular liver cancer; prostate cancer; rectal cancer; renal cancer; renal cell (kidney) cancer; renal cell cancer; respiratory tract cancer; retinoblastoma; rhabdomyosarcoma; salivary gland cancer; Sezary syndrome; small cell lung cancer; small intestine cancer; soft tissue sarcoma; squamous cell carcinoma; squamous neck cancer; stomach (gastric) cancer; supratentorial primitive neuroectodermal tumors; T-cell lymphoma; testicular cancer; throat cancer; thymic carcinoma; thymoma; thyroid cancer; transitional cell cancer; transitional cell cancer of the renal pelvis and ureter; trophoblastic tumor; ureter cancer; urethral cancer; uterine cancer; uterine sarcoma; vaginal cancer; vulvar cancer; Waldenstrom's macroglobulinemia; or Wilm's tumor. The premalignant condition can include without limitation Barrett's Esophagus. The autoimmune disease can include without limitation one of inflammatory bowel disease (IBD), Crohn's disease (CD), ulcerative colitis (UC), pelvic inflammation, vasculitis, psoriasis, diabetes, autoimmune hepatitis, multiple sclerosis, myasthenia gravis, Type I diabetes, rheumatoid arthritis, psoriasis, systemic lupus erythematosis (SLE), Hashimoto's Thyroiditis, Grave's disease, Ankylosing Spondylitis Sjogren's Disease, CREST syndrome, Scleroderma, Rheumatic Disease, organ rejection, Primary Sclerosing Cholangitis, or sepsis. The cardiovascular disease can include without limitation one of atherosclerosis, congestive heart failure, vulnerable plaque, stroke, ischemia, high blood pressure, stenosis, vessel occlusion or a thrombotic event. The neurological disease can include without limitation one of Multiple Sclerosis (MS), Parkinson's Disease (PD), Alzheimer's Disease (AD), schizophrenia, bipolar disorder, depression, autism, Prion Disease, Pick's disease, dementia, Huntington disease (HD), Down's syndrome, cerebrovascular disease, Rasmussen's encephalitis, viral meningitis, neurospsychiatric systemic lupus erythematosus (NPSLE), amyotrophic lateral sclerosis, Creutzfeldt-Jacob disease, Gerstmann-Straussler-Scheinker disease, transmissible spongiform encephalopathy, ischemic reperfusion damage (e.g. stroke), brain trauma, microbial infection, or chronic fatigue syndrome. The pain can include without limitation one of fibromyalgia, chronic neuropathic pain, or peripheral neuropathic pain. The infectious disease can include without limitation one of a bacterial infection, viral infection, yeast infection, Whipple's Disease, Prion Disease, cirrhosis, methicillin-resistant Staphylococcus aureus, HIV, Hepatitis C virus (HCV), Epstein Barr virus, Helicobacter pylori, hepatitis, syphilis, meningitis, malaria, tuberculosis, or influenza.

### Therapy

Any therapy (e.g., therapeutic or prophylactic agent) which is useful, has been used, is currently being used, or may be used for the prevention, treatment and/or management of neoplasia or cancer can be used to prevent, treat, and/or manage a patient with the compositions and methods as disclosed herein, for example with compositions and methods for the administration of the compounds as disclosed herein, administered sequentially, concurrently in separate dosage forms, or in the same dosage form, for the treatment and/or prevention of, for example, neoplasia or cancer. Therapies (e.g., therapeutic or prophylactic agents) include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, anti-angiogenesis therapies, targeted therapies, and/or biological therapies including immunotherapies and surgery. In certain embodiments, a prophylactically and/or therapeutically effective regimen comprises the administration of a combination of therapies. In certain embodiments, the compositions as disclosed herein, combinations thereof, and pharmaceutically acceptable salts thereof can be administered as an agent to treat or prevent neoplasia.

Examples of cancer therapies which may be used in conjunction with the compositions and methods as disclosed herein include, but are not limited to, biologics such as Rituxan (rituximab), Herceptin (trastuzumab), Erbitux (cetuximab), Vectibix (Panitumumab), Arzerra (Ofatumumab), Benlysta (belimumab), Yervoy (ipilimumab), Perjeta (Pertuzumab), Tremelimumab, Opdivo (Nivolumab), Keytruda (pembrolizumab), Dacetuzumab, Urelumab, MPDL3280A, Lambrolizumab, Blinatumomab, Humira (adalimumab), Campath (Alemtuzumab), CEA-Scan Arcitumomab (fab fragment), Erbitux (Cetuximab), Myoscint (Imciromab Pentetate), ProstaScint (Capromab Pendetide), Remicade (Infliximab), ReoPro (Abciximab), Simulect (Basiliximab), Synagis (Palivizumab), Verluma (Nofetumomab), Xolair (Omalizumab), Zenapax (Daclizumab), Zevalin (Ibritumomab Tiuxetan), Orthoclone OKT3 (Muromonab-CD3), Panorex (Edrecolomab), Mylotarg (Gemtuzumab ozogamicin), golimumab (Centocor), Cimzia (Certolizumab pegol), Soliris (Eculizumab), CNTO 1275 (ustekinumab), Vectibix (panitumumab), Bexxar (tositumomab and ¹³¹I tositumomab), Avastin, and combinations thereof.

Examples of cancer therapies which may be used in conjunction with the compositions and methods as disclosed herein include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandornate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents, dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; histone deacetylase inhibitors (HDAC-Is) hydroxyurea; idarubicin hydrochloride; Ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-Ia; interferon gamma-Ib; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mifepristone; mitindomide; mitocarcin; mitocromin; mitogillin; mitomalcin; mitomycin; mitosper; mitotane; mitoxantrone hydrochloride; mycophenolic acid; nocodazole; nogalamycin; ORG 34517; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; RU486; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies which may be used in conjunction with the compositions and methods as disclosed herein include, but are not limited to: 20-epi-1,25 dihydroxyvitamin D3; 5-ethynyluraci1; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-I; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin alpha v beta 3 antibodies); vorozole; zanoterone; zeniplatin; zilascorb; and zinostatin stimalamer.

Any suitable mode of administration may be used in accordance with the present invention including but not limited to parenteral administration such as intravenous, subcutaneous, intramuscular and intrathecal administration; oral, intranasal, rectal or vaginal administration may also be used; directly into the tumor; transdermal patches; implant devices (particularly for slow release); finally, topical administration may be used. The mode of administration will vary according to the disease to be treated. In one embodiment, the arsenic trioxide of the invention is dissolved in an aqueous solution of sodium hydroxide, with the pH adjusted to a physiologically acceptable range, e.g. about pH 6-8.

A non-limiting list of compounds that could be used in conjunction with the compositions and methods as disclosed herein includes: inhibitors of interleukin-3 receptor (IL-3R) and CD123 (including peptides, peptide-conjugates, antibodies, antibody-conjugates, antibody fragments, and antibody fragment-conjugates that target IL-3R or CD123); cantharidin; norcantharidin and analogs and derivatives thereof, Notch pathway inhibitors including gamma secretase inhibitors; sonic hedgehog/smoothened pathway inhibitors including cyclopamine and analogs thereof, antibodies to CD96; certain NF-kappaB/proteasome inhibitors including parthenolide and analogs thereof; certain triterpenes including celastrol; certain mTOR inhibitors; compounds and antibodies that target the urokinase receptor; sinefungin; certain inosine monophosphate dehydrogenase (IMPDH) inhibitors; PPAR-alpha and PPAR-gamma agonists and antagonists (including pioglitazone, tesaslitazar, muraglitazar, peliglitazar, lobeglitazone, balaglitazone, ragaglitazar, rosiglitazone, farglitazar, sodeiglitazar, reglitazar, naveglitazar, oxeglitazar, metaglidasen, netoglitazone, darglitazone, englitazone, thiazolidinediones, aleglitazar, edaglitazone, rivoglitazone, troglitazone, imiglitazar, and sipoglitazar); telomerase inhibitors; antibodies to EpCAM (ESA); GSK-3 beta agonists and antagonists (including Lithium, 6-bromoinirubin-3′-oxime (BIO), TDZD8); Wnt pathway inhibitors including antibodies to frizzled or small molecules that inhibit disheveled/frizzled or beta catenin; anti-CD20 antibodies and conjugates (e.g. Rituxan, Bexxar, Zevalin) for novel use in multiple myeloma or melanoma; anti-CD133 antibody; anti-CD44 antibody; antibodies to IL-4; certain differentiation agents such as versnarinone; compounds that target CD33 such as an antibody or betulinic acid; compounds that target lactadherin such as an antibody; small molecules or antibodies that target CXCR4 or SDF-1; small molecules or antibodies that target multi-drug resistance pumps; inhibitors of survivin; inhibitors of XIAP; small molecules that target Bcl-2; antibodies to CLL-1; and furin inhibitors (such as cucurbitacins).

An additional non-limiting list of compounds which may be used in conjunction with the compositions and methods as disclosed herein to target cancer includes: i) antibodies, antibody fragments, and proteins that are either naked or conjugated to a therapeutic moiety that target certain cell surface targets on cancer stem cells, or ii) small molecules known in the art including ones that can be further optimized (e.g., via chemistry) or identified via a cancer stem cell-based screen (e.g., such as one that would determine whether a compound impairs proliferation or viability of a cancer stem cell through standard methods, the cell surface and intracellular targets including (not meant to be exhaustive) are: Rex1 (Zfp42), CTGF, Activin A, Wnt, FGF-2, HIF-1, AP-2gamma, Bmi-1, nucleostemin, hiwi, Moz-TIF2, Nanog, beta-arrestin-2, Oct-4, Sox2, stella, GDF3, RUNX3, EBAF, TDGF-1, nodal, ZFPY, PTNE, Evi-1, Pax3, Mcl-1, c-kit, Lex-1, Zfx, lactadherin, aldehyde dehydrogenase, BCRP, telomerase, CD133, Bcl-2, CD26, Gremlin, and FoxC2.

In some embodiments, the therapy(ies) is an immunomodulatory agent which may be used in conjunction with the compositions and methods as disclosed herein. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamides (e.g., leflunamide), T cell receptor modulators, cytokine receptor modulators, and modulators mast cell modulators. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent. In some embodiments, the therapy(ies) is an anti-angiogenic agent. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that specifically bind to TNF-alpha, nucleic acid molecules (e.g., antisense molecules or triple helices), organic molecules, inorganic molecules, and small molecules that reduce or inhibit angiogenesis.

In certain embodiments, the therapy(ies) is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, mephalen, and themozolomide. Nitrosoureas include but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine). In some embodiments of the invention, the therapy(ies) includes the administration cantharidin or an analog thereof. The invention includes the use of agents that target cancer stem cells. In certain embodiments, the agent acts alone. In other embodiments, the agent is attached directly or indirectly to another therapeutic moiety. Non-limiting examples of therapeutic moieties include, but are not limited to alkylating agents, anti-metabolites, plant alkaloids, cytotoxic agents, chemotherapeutic agents (e.g., a steroid, cytosine arabinoside, fluoruracil, methotrexate, aminopterin, mitomycin C, demecolcine, etoposide, mithramycin, calicheamicin, CC-1065, chlorambucil or melphalan), radionuclides, therapeutic enzymes, cytokines, toxins including plant-derived toxins, fungus-derived toxins, bacteria-derived toxin (e.g., deglycosylated ricin A chain, a ribosome inactivating protein, alpha-sarcin, aspergillin, restirictocin, a ribonuclease, a diphtheria toxin, Pseudomonas exotoxin, a bacterial endotoxin or the lipid A moiety of a bacterial endotoxin), growth modulators and RNase. In some embodiments, the agent used is an agent that binds to a marker, e.g., an antigen on a cancer stem cell. In a specific embodiment, the agent binds to an antigen that is expressed at a greater level on cancer stem cells than on normal stem cells. In a specific embodiment, the agent binds specifically to a cancer stem cell antigen that is not a normal stem cell. In other embodiments, the therapy(ies) is an agent that binds to a marker on cancer stem cells. In one embodiment, the agent that binds to a marker on cancer stem cells is an antibody or an antibody conjugated to a therapeutic moiety or an antibody fragment conjugated to a therapeutic moiety.

In certain embodiments, antibodies or fragments thereof that bind to a marker on cancer stem cells are substantially non-immunogenic in the treated subject. Methods for obtaining non-immunogenic antibodies include, but are not limited to, chimerizing the antibody, humanizing the antibody, and isolating antibodies from the same species as the subject receiving the therapy. Antibodies or fragments thereof that bind to markers in cancer stem cells can be produced using techniques known in the art.

In some embodiments, the therapy comprises the use of X-rays, gamma rays and other sources of radiation to destroy cancer stem cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer stem cells, cancer cells and/or a tumor mass.

In some embodiments, the therapy used is a proliferation based therapy. Non-limiting examples of such therapies include a chemotherapy and radiation therapy as described supra.

Currently available therapies and their dosages, routes of administration and recommended usage are known in the art and have been described in such literature as the Physician's Desk Reference (60th ed., 2006).

In a specific embodiment, cycling therapy involves the administration of a first cancer therapeutic for a period of time, followed by the administration of a second cancer therapeutic for a period of time, optionally, followed by the administration of a third cancer therapeutic for a period of time and so forth, and repeating this sequential administration, i.e., the cycle in order to reduce the development of resistance to one of the cancer therapeutics, to avoid or reduce the side effects of one of the cancer therapeutics, and/or to improve the efficacy of the cancer therapeutics.

When two prophylactically and/or therapeutically effective regimens are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the cancer therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

### Packaging/Treatment Kits

The disclosure provides a kit for conveniently and effectively carrying out the methods in accordance with the present disclosure. Such kits may be suited for the delivery of, for example, solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists of two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the invention are contained in-between a card and clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when dosage has been taken.

The invention will be illustrated in more detail with reference to the following Examples, but it should be understood that the present invention is not deemed to be limited thereto.

### Aspects

Aspect 1: A compound having the structure of Formula I:

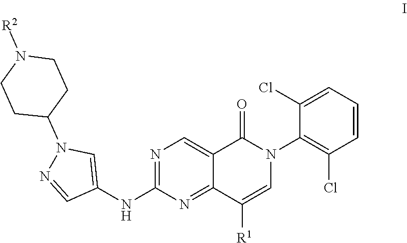

wherein: R¹ is halo, C₁₋₆ alkyl, C₃₋₈ cycloalkyl or C₂₋₆ alkenyl; and R² is H, C₁₋₆ alkyl, or C₃₋₈ cycloalkyl; or a pharmaceutically acceptable salt thereof.

Aspect 2: The compound of Aspect 1, wherein R¹ is halo.

Aspect 3: The compound of Aspect 1 or 2, wherein R¹ is chloro, bromo, fluoro, or iodo.

Aspect 4: The compound of Aspect 1 or 2, wherein R¹ is chloro or bromo.

Aspect 5: The compound of Aspect 1, wherein R¹ is C₁₋₄ alkyl.

Aspect 6: The compound of Aspect 1 or 5, wherein R¹ is methyl, ethyl, n-propyl or i-propyl.

Aspect 7: The compound of Aspect 1, wherein R¹ is C₃₋₇ cycloalkyl.

Aspect 8: The compound of Aspect 1 or 7, wherein R¹ is cyclopropyl.

Aspect 9: The compound of Aspect 1, wherein R¹ is C₁₋₄ alkenyl.

Aspect 10: The compound of Aspect 1 or 9, wherein R¹ is vinyl or isopropenyl.

Aspect 11: The compound of any one of the preceding Aspects, wherein R² is C₁₋₆alkyl.

Aspect 12: The compound of any one of the preceding Aspects, wherein R² is methyl, ethyl, propyl, butyl, pentyl, or hexyl.

Aspect 13: The compound of any one of the preceding Aspects, wherein R² is methyl, ethyl, n-propyl, or i-propyl.

Aspect 14: The compound of any one of Aspects 1 to 10, wherein R² is C₃-8cycloalkyl.

Aspect 15: The compound of any one of Aspects 1 to 10 or 14, wherein R² is cyclopropyl, cyclobutyl, cyclopentyl, or cyclohexyl.

Aspect 16: The compound of any one of Aspects 1 to 10, 14, or 15, wherein R² is cyclopropyl.

Aspect 17: The compound of Aspect 1, having the structure of Formula II:

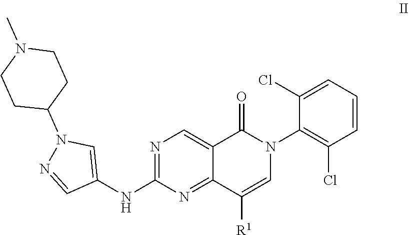

or a pharmaceutically acceptable salt thereof.

Aspect 18: The compound of Aspect 1 that is:

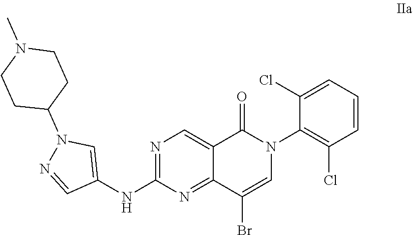 [SMILES: CN1CCC(n2cc(Nc3ncc4c(=O)n(-c5c(Cl)cccc5Cl)cc(Br)c4n3)cn2)CC1]

or a pharmaceutically acceptable salt thereof.

Aspect 19: A compound having the structure of Formula III:

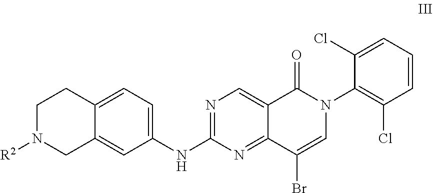

wherein: R² is H, C₁₋₆alkyl, or C₃₋₈ cycloalkyl, or a pharmaceutically acceptable salt thereof.

Aspect 20: The compound of Aspect 19, wherein R² is H.

Aspect 21: The compound of Aspect 19, wherein R² is C₁₋₆alkyl.

Aspect 22: The compound of Aspect 19, wherein R² is methyl.

Aspect 23: A compound having the structure of Formula IV:

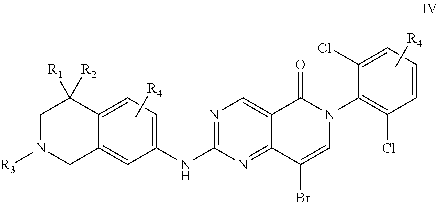

wherein R1, R2 is H; Methyl; Ethyl; or Propyl; optionally wherein R1 and R2 are connected to form a 3-6 membered ring; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

Aspect 24: A compound having the structure of Formula V:

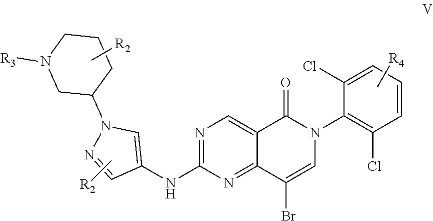

wherein R2 is H; Methyl; Ethyl; or Propyl; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

Aspect 25: A compound having the structure of Formula VI:

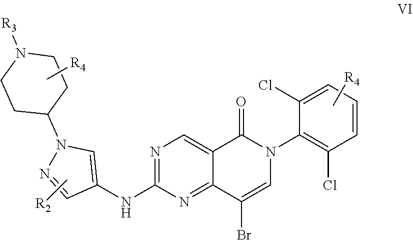

wherein R2 is H; Methyl; Ethyl; or Propyl; R3 is C1-4 small alkyl; or C3-6 cycloalkyl ring; R4 is H; C1-3 alkyl; CF3; OMethyl; OCF3; OCF2H; CN; or halo, or a pharmaceutically acceptable salt thereof.

Aspect 26: A pharmaceutical composition comprising one or more compound of any one of the preceding Aspects and one or more pharmaceutically acceptable excipient.

Aspect 27: A method for inhibiting Wee1 in a patient in need of such treatment, comprising administering the compound or composition of any one of Aspects 1 to 26 to the patient.

Aspect 28: A method of treating cancer in a patient need of such treatment, comprising administering to the patient the compound of any one of Aspects 1 to 26 to the patient.

Aspect 29: The method of Aspect 28, wherein the cancer is adrenocortical carcinoma, an AIDS-related cancer (such as an AIDS-related lymphoma), anal cancer, appendix cancer, astrocytomas, atypical teratoid/rhabdoid tumor, basal cell carcinoma, bile duct cancer, bladder cancer, bone cancer, brain cancer (such as glioblastoma), breast cancer, bronchial tumor, cancer of unknown primary site such as carcinoma of unknown primary site, carcinoid tumor, castration-resistant prostate cancer, central nervous system cancer (such as central nervous system atypical teratoid/rhabdoid tumor, central nervous system embryonal tumors, central nervous system lymphoma, primary central nervous system lymphoma), cervical cancer, chordoma, chondrosarcoma, chronic myeloproliferative disorders, colon cancer, colorectal cancer, craniopharyngioma, desmoplastic round cell tumor, diffuse large B-cell lymphoma, endometrial cancer, ependymoblastoma, ependymoma, esophageal cancer, Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor, germ cell tumor, gestational trophoblastic tumor, glioma, head cancer, hepatocellular (liver) cancer, high grade prostate cancer, histiocytosis, hypopharyngeal cancer, Kaposi sarcoma, kidney (renal) cancer, Langerhans cell histiocytosis, laryngeal cancer, leptomeningeal disease, lip cancer, low grade prostate cancer, leukemia (such as chronic lymphocytic leukemia, chronic myelogenous leukemia, hairy cell leukemia,), lung cancer, lymphoma (such as Burkitt lymphoma, central nervous system lymphoma, T-Cell lymphoma such as cutaneous T-Cell lymphoma, Hodgkin's lymphoma, Non-Hodgkin's lymphoma, lymphoplasmacytic lymphoma), medium grade prostate cancer, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, multiple myeloma/plasma cell neoplasm, mycosis fungoides, myelodysplastic syndromes, myelodysplastic/myeloproliferative neoplasms, myeloproliferative disorder, nasal cavity or paranasal sinus cancer, nasopharyngeal cancer, neck cancer, neuroblastoma, ocular cancer, ocular melanoma, oral cancer, oropharyngeal cancer, oral cavity cancer, osteosarcoma or malignant fibrous histiocytoma of bone, osteosarcoma or malignant fibrous histiocytoma, ovarian cancer, ovarian germ cell tumor, ovarian epithelial cancer, ovarian low malignant potential tumor, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, pregnancy cancer, prostate cancer, rectal cancer, renal pelvis cancer, respiratory tract carcinoma involving the NUT gene on chromosome 15, retinoblastoma, rhabdomyosarcoma, salivary gland cancer, sarcoma, Sezary syndrome, skin cancer, skin carcinoma, small intestine cancer, soft tissue sarcoma, spinal cord tumor, squamous cell carcinoma, squamous neck cancer with occult primary, supratentorial primitive neuroectodermal tumors, testicular cancer, throat cancer, thymoma or thymic carcinoma, thyroid cancer, transitional cell cancer of the renal pelvis or ureter, unusual cancers of childhood, ureter cancer, urethral cancer, uterine sarcoma, vaginal cancer, vulvar cancer, Waldenstrom's macroglobulinemia (lymphoplasmacytic lymphoma), Wilm's tumor, or women's cancer.

Aspect 30: The method of Aspect 29, wherein the cancer is breast cancer, prostate cancer, pancreatic cancer, lung cancer, colorectal cancer, ovarian cancer, liver cancer, melanoma, renal cancer, a central nervous system cancer, brain cancer such as glioblastoma, a leukemia, or a lymphoma.

Aspect 31: The method of any one of Aspects 27 to 30, wherein the compound is administered in combination with at least one additional therapeutic agent.

Aspect 32: The method of Aspect 31, wherein the at least one additional therapeutic agent is a chemotherapeutic.

Aspect 33: The method of Aspect 32, wherein said chemotherapeutic agent is selected from the group consisting of: busulfan, melphalan, chlorambucil, cyclophosphamide, ifosfamide, temozolomide, bendamustine, cis-platin, mitomycin C, bleomycin, carboplatin, camptothecin, irinotecan, topotecan, doxorubicin, epirubicin, aclarubicin, mitoxantrone, elliptinium, etoposide, 5-azacytidine, gemcitabine, 5-fluorouracil, methotrexate, 5-fluoro-2′-deoxy-uridine, fludarabine, nelarabine, ara-C, pralatrexate, pemetrexed, hydroxyurea, thioguanine, colchicine, vinblastine, vincristine, vinorelbine, paclitaxel, ixabepilone, cabazitaxel, docetaxel, campath, panitumumab, metazotuzumab, navuzumab, pymzumab, remoluzumab, bevacizumab, partuzumab, trastuzumab, cetuximab, obinutuzumab, olfamzumab, rituximab, alemtuzumab, tiemuzumab, toximab, bentuximab, daremuzumab, errotuzumab, T-DM1, ofatumumab, dinutuximab, blinatumomab, ipilimma, avastin, trastuzumab, rituximab, imatinib, gefitinib, erlotinib, osimertinib, afatinib, ceritinib, aletinib, crizotinib, erlotinib, lapatinib, sorafenib, sunitinib, nilotinib, dasatinib, pazopanib, temsirolimus, everolimus, vorinostat, romidepsin, panobinostat, belinostat, tamoxifen, letrozole, fulvestrant, mitoguazone, octreotide, retinoic acid, arsenic trioxide, zoledronic acid, bortezomib, carfilzomib, ixazomib, vismodegib, sonidegib, denosumab, thalidomide, lenalidomide, venetoclax, aldesleukin (recombinant human interleukin-2), sipueucel-T (prostate cancer therapeutic vaccine), palbociclib, olaparib, niraparib, rucaparib, talazoparib, and combinations thereof.

Aspect 34: A method for reducing the activity of a kinase encoded by the gene WEE1, the method comprising contacting the kinase with an inhibitory amount of a compound of any one of Aspects 1-26.

Aspect 35: A method according to Aspect 34, wherein the method is carried out in vitro.

Aspect 36: A method according to Aspect 34, wherein the method is carried out in a subject.

Aspect 37: A method for treating or preventing Wee1-mediated disease in a subject in need thereof comprising administering the subject an effective amount of a compound of any one of Aspects 1-26, or a pharmaceutically acceptable salt or prodrug thereof, or a pharmaceutical composition thereof.

Aspect 38. The method of Aspect 37, wherein the disease is a cancer selected from adrenocortical carcinoma, an AIDS-related cancer (such as an AIDS-related lymphoma), anal cancer, appendix cancer, astrocytomas, atypical teratoid/rhabdoid tumor, basal cell carcinoma, bile duct cancer, bladder cancer, bone cancer, brain cancer (such as glioblastoma), breast cancer, bronchial tumor, cancer of unknown primary site such as carcinoma of unknown primary site, carcinoid tumor, castration-resistant prostate cancer, central nervous system cancer (such as central nervous system atypical teratoid/rhabdoid tumor, central nervous system embryonal tumors, central nervous system lymphoma, primary central nervous system lymphoma), cervical cancer, chordoma, chondrosarcoma, chronic myeloproliferative disorders, colon cancer, colorectal cancer, craniopharyngioma, desmoplastic round cell tumor, diffuse large B-cell lymphoma, endometrial cancer, ependymoblastoma, ependymoma, esophageal cancer, Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor, germ cell tumor, gestational trophoblastic tumor, glioma, head cancer, hepatocellular (liver) cancer, high grade prostate cancer, histiocytosis, hypopharyngeal cancer, Kaposi sarcoma, kidney (renal) cancer, Langerhans cell histiocytosis, laryngeal cancer, leptomeningeal disease, lip cancer, low grade prostate cancer, leukemia (such as chronic lymphocytic leukemia, chronic myelogenous leukemia, hairy cell leukemia,), lung cancer, lymphoma (such as Burkitt lymphoma, central nervous system lymphoma, T-Cell lymphoma such as cutaneous T-Cell lymphoma, Hodgkin's lymphoma, Non-Hodgkin's lymphoma, lymphoplasmacytic lymphoma), medium grade prostate cancer, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, multiple myeloma/plasma cell neoplasm, mycosis fungoides, myelodysplastic syndromes, myelodysplastic/myeloproliferative neoplasms, myeloproliferative disorder, nasal cavity or paranasal sinus cancer, nasopharyngeal cancer, neck cancer, neuroblastoma, ocular cancer, ocular melanoma, oral cancer, oropharyngeal cancer, oral cavity cancer, osteosarcoma or malignant fibrous histiocytoma of bone, osteosarcoma or malignant fibrous histiocytoma, ovarian cancer, ovarian germ cell tumor, ovarian epithelial cancer, ovarian low malignant potential tumor, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, pregnancy cancer, prostate cancer, rectal cancer, renal pelvis cancer, respiratory tract carcinoma involving the NUT gene on chromosome 15, retinoblastoma, rhabdomyosarcoma, salivary gland cancer, sarcoma, Sezary syndrome, skin cancer, skin carcinoma, small intestine cancer, soft tissue sarcoma, spinal cord tumor, squamous cell carcinoma, squamous neck cancer with occult primary, supratentorial primitive neuroectodermal tumors, testicular cancer, throat cancer, thymoma or thymic carcinoma, thyroid cancer, transitional cell cancer of the renal pelvis or ureter, unusual cancers of childhood, ureter cancer, urethral cancer, uterine sarcoma, vaginal cancer, vulvar cancer, Waldenstrom's macroglobulinemia (lymphoplasmacytic lymphoma), Wilm's tumor, or women's cancer.

### EXAMPLES

### Preparation of Intermediary Compounds

### Example 1: 1-(1-methyl-4-piperidyl)pyrazol-4-amine (IM1)

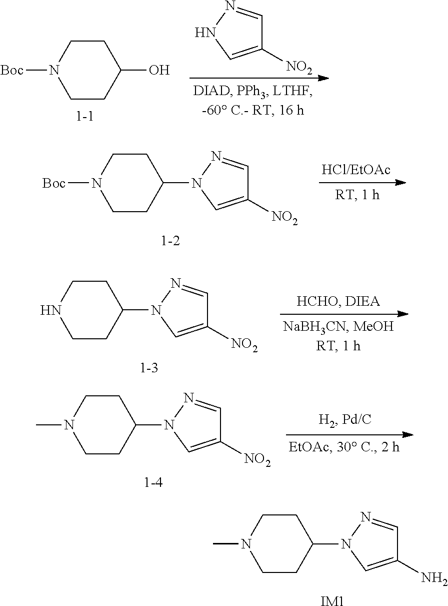

Step 1: To a solution of 4-nitro-1H-pyrazole (10 g, 88.44 mmol, 1.0 eq.), tert-butyl 4-hydroxypiperidine-1-carboxylate (1-1, 17.80 g, 88.44 mmol, 1.0 eq.) and PPh₃ (34.79 g, 132.66 mmol, 1.5 eq.) in THF (400 mL) was added DIAD (26.82 g, 132.66 mmol, 1.5 eq.) dropwise at −60° C. under N₂. The mixture was allowed to warm to 20° C. and stirred for 16 hrs. Then the mixture was diluted with EtOAc (200 mL), washed with water (200 mL)/brine (200 mL), dried, filtered and concentrated. The residue was purified through silica gel column chromatography to give tert-butyl 4-(4-nitropyrazol-1-yl)piperidine-1-carboxylate (1-2, 25 g) as a white solid. ESI [M+H-100]=197.3

Step 2: A solution of tert-butyl 4-(4-nitropyrazol-1-yl)piperidine-1-carboxylate (1-2, 10 g, 33.75 mmol) in HCl/EtOAc (4 M, 100 mL) was stirred at 20° C. for 1 hr and then concentrated to give 4-(4-nitropyrazol-1-yl)piperidine (1-3) as a white solid. ESI [M+H]=197.3

Step 3: To a solution of 4-(4-nitropyrazol-1-yl)piperidine (1-3, 8.2 g, 35.24 mmol, HCl salt) in MeOH (100 mL) was added DIEA (9.11 g, 70.49 mmol, 2.0 eq.) and formaldehyde (1.59 g, 52.87 mmol, 1.5 eq.). The mixture was stirred at 20° C. for 15 min. Then NaBH₃CN (4.43 g, 70.49 mmol, 2.0 eq.) was added and the mixture was stirred at 20° C. for another 45 min. The reaction mixture was diluted with H₂O (300 mL) and extracted with EtOAc (500 mL×2). The combined organic layers were dried over Na₂SO₄, filtered and concentrated. The residue was purified through silica gel column chromatography to give 1-methyl-4-(4-nitropyrazol-1-yl)piperidine (1-4, 2 g) as a white solid. ESI [M+H]=211.2.

Step 4: A mixture of 1-methyl-4-(4-nitropyrazol-1-yl)piperidine (1-4, 2 g, 9.51 mmol) and Pd/C (2 g, 10% purity) in EtOAc (20 mL) was degassed and purged with H₂ for 3 times, and then the mixture was stirred at 30° C. for 2 hrs under H₂ (15 Psi). The reaction mixture was filtered and concentrated to give 1-(1-methyl-4-piperidyl)pyrazol-4-amine (IM1, 1.8 g, crude) as a pink oil. ESI [M+H]=181.2.

### Example 2: tert-butyl 4-(4-amino-1H-pyrazol-1-yl)piperidine-1-carboxylate (IM2)

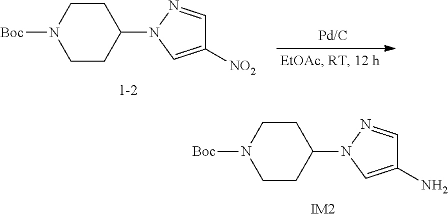

A mixture of tert-butyl 4-(4-nitropyrazol-1-yl)piperidine-1-carboxylate (1-2, 3 g, 10.12 mmol) and Pd/C (1 g, 10% purity) in EtOAc (30 mL) was stirred at 20° C. for 12 h under H₂ (15 psi). The mixture was filtered and concentrated to give tert-butyl 4-(4-aminopyrazol-1-yl)piperidine-1-carboxylate (IM2, 1.4 g) as a brown oil. ESI [M+H]=267.2.

### Example 3: 8-bromo-6-(2,6-dichlorophenyl)-2-(methylthio)pyrido[4,3-d]pyrimidin-5(6H)-one (IM3)

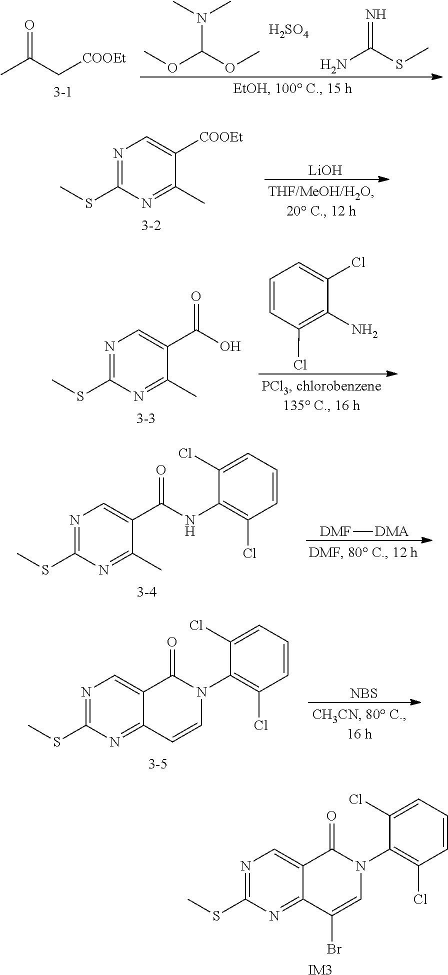

Step 1: A mixture of 2-methylisothiourea; sulfuric acid (28.88 g, 103.74 mmol, 0.5 eq.) and 1,1-dimethoxy-N,N-dimethyl-methanamine (3-1, 24.72 g, 207.47 mmol) was heated to 100° C. for 3 h. The mixture was cooled to 20° C. and a solution of ethyl 3-oxobutanoate (27 g, 207.47 mmol, 1 eq.) in EtOH (120 mL) was added and the resulting mixture was heated at 100° C. for 12 h, cooled to rt and concentrated. The residue was diluted with EtOAc (1.0 L), washed with water (500 mL)/brine (500 mL), dried and concentrated to give ethyl 4-methyl-2-methylsulfanyl-pyrimidine-5-carboxylate (3-2, 41.12 g) as a yellow oil. ESI [M+H]=213.1.

Step 2: A solution of LiOH·H₂O (15 g, 357.45 mmol, 1.85 eq.) in H₂O (150 mL) was added into a solution of ethyl 4-methyl-2-methylsulfanyl-pyrimidine-5-carboxylate (3-2, 41.12 g, 193.72 mmol) in THE (150 mL)/MeOH (50 mL). The mixture was stirred at 20° C. for 12 h and concentrated to remove MeOH and THF. The residual aqueous layer was extracted with methyl tert-butyl ether (300 mL). The aqueous layer was acidified with 1N HCl to adjust pH=2. The precipitate was collected and dried to give 4-methyl-2-methylsulfanyl-pyrimidine-5-carboxylic acid (3-3, 24.4 g) as a yellow solid. ¹H NMR (400 MHz, DMSO-d₆) δ=8.88 (s, 1H), 2.66 (s, 3H), 2.54 (s, 3H).

Step 3: A mixture of 4-methyl-2-methylsulfanyl-pyrimidine-5-carboxylic acid (3-3, 18.2 g, 98.80 mmol) and 2,6-dichloroaniline (16.01 g, 98.80 mmol, 1 eq.) in chlorobenzene (455 mL) was degassed and purged with N₂ for 3 times. PCl₃ (13.57 g, 98.80 mmol, 1 eq.) was added and the mixture was stirred at 135° C. for 16 h under N₂. The mixture was concentrated and the residue was poured into saturated aqueous Na₂CO₃ solution (500 mL) and extracted with 2-methyl-THF (1 L)/EtOAc (500 mL). The organic phase was dried and concentrated. The residue was triturated with EtOAc (100 mL) to give N-(2,6-dichlorophenyl)-4-methyl-2-methylsulfanyl-pyrimidine-5-carboxamide (3-4, 18.0 g) as a yellow solid. ¹H NMR (400 MHz, DMSO-d₆) δ=10.54 (s, 1H), 8.74 (s, 1H), 7.61 (d, J=8.00 Hz, 2H), 7.40-7.45 (m, 1H), 2.61 (s, 3H), 2.57 (s, 3H). ESI [M+H]=328.0/330.0.

Step 4: To a solution of N-(2,6-dichlorophenyl)-4-methyl-2-methylsulfanyl-pyrimidine-5-carboxamide (3-4, 15 g, 45.70 mmol) in DMF (150 mL) was added DMF-DMA (16.34 g, 137.10 mmol, 3 eq.). The resulting mixture was stirred at 80° C. for 12 h, cooled to rt and concentrated. The residue was triturated with EtOH (50 mL) to give 6-(2,6-dichlorophenyl)-2-methylsulfanyl-pyrido[4,3-d]pyrimidin-5-one (3-5, 5.3 g) as a white solid. ESI [M+H]=338.1/340.1.

Step 5: To a solution of 6-(2,6-dichlorophenyl)-2-methylsulfanyl-pyrido[4,3-d]pyrimidin-5-one (3-5, 3.88 g, 11.47 mmol) in MeCN (56 mL) was added NBS (3.06 g, 17.21 mmol, 1.5 eq.) and the mixture was stirred at 80° C. for 16 h. The mixture was concentrated and the residue was triturated with EtOH (20 mL) to give 8-bromo-6-(2,6-dichlorophenyl)-2-methylsulfanyl-pyrido[4,3-d]pyrimidin-5-one (IM3, 3.1 g, as a light yellow solid. ESI [M+H]=417.9/415.9.

### Example 4: 8-bromo-6-(2,6-dichlorophenyl)-2-(methylsulfinyl)pyrido[4,3-d]pyrimidin-5(6H)-one (IM4)

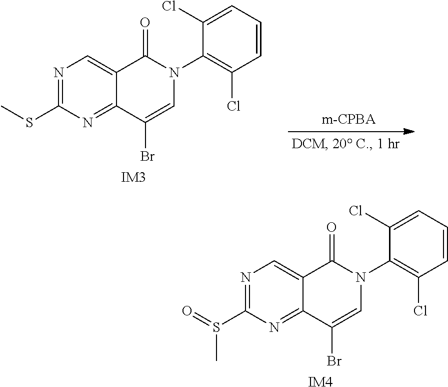

To a solution of 8-bromo-6-(2,6-dichlorophenyl)-2-methylsulfanylpyrido[4,3-d]pyrimidin-5-one (IM3, 100 mg, 239.75 μmol) in DCM (5 mL) was added m-CPBA (62.06 mg, 359.62 μmol, 85% purity, 1.5 eq.). The mixture was stirred at 20° C. for 1 h and then concentrated to give IM4 for use into next step without further purification. ESI [M+H]=433.9/431.9

### Example 5: tert-butyl 4-(4-((8-bromo-6-(2,6-dichlorophenyl)-5-oxo-5,6-dihydropyrido[4,3-d]pyrimidin-2-yl)amino)-1H-pyrazol-1-yl)piperidine-1-carboxylate (IM5)

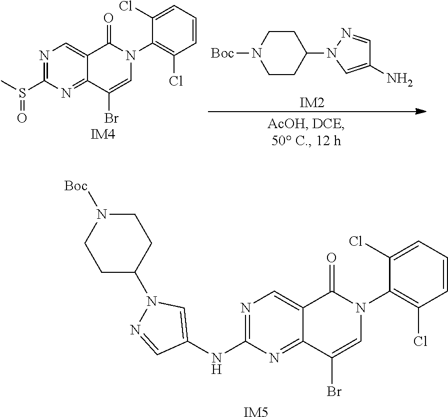

To a solution of 8-bromo-6-(2,6-dichlorophenyl)-2-methylsulfinylpyrido[4,3-d]pyrimidin-5-one (IM4, 1 g, 2.31 mmol) in DCE (50 mL)/DCM (50 mL) was added tert-butyl 4-(4-aminopyrazol-1-yl)piperidine-1-carboxylate (IM2, 1.23 g, 4.62 mmol, 2 eq.), followed by addition of AcOH (138.65 mg, 2.31 mmol, 1 eq.). The mixture was stirred at 50° C. for 12 h. The mixture was concentrated and the residue was purified by reverse phase prep-HPLC to give tert-butyl 4-[4-[[8-bromo-6-(2,6-dichlorophenyl)-5-oxo-pyrido[4,3d]pyrimidin-2-yl]amino]pyrazol-1-yl]piperidine-1-carboxylate (IM5, 1.4 g) as a yellow solid. ESI [M+H-56]=580.1/578.2.

### Example 6: tert-butyl 4-[4-[[6-(2,6-dichlorophenyl)-8-isopropenyl5-oxo-pyrido[4,3-d]pyrimidin-2-yl]amino]pyrazol-1-yl]piperidine-1-carboxylate (IM6)

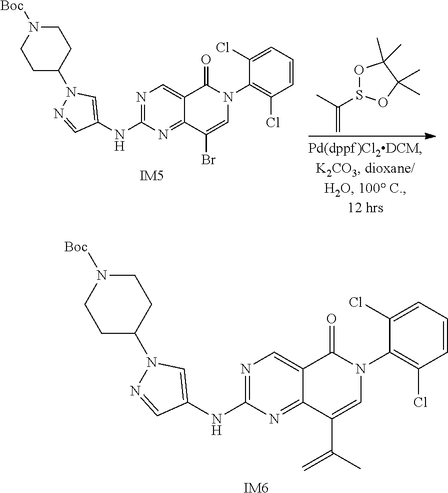

A mixture of tert-butyl 4-[4-[[8-bromo-6-(2,6-dichlorophenyl)-5-oxo-pyrido[4,3-d]pyrimidin-2-yl]amino]pyrazol-1-yl]piperidine-1-carboxylate (IM5, 200 mg, 314.79 μmol), 2-isopropenyl-4,4,5,5-tetramethyl-1,3,2-dioxaborolane (105.80 mg, 629.58 μmol, 2 eq.), Pd(dppf)Cl₂·DCM (128.53 mg, 157.40 μmol, 0.5 eq.) and K₂CO₃ (87.01 mg, 629.58 μmol, 2 eq.) in dioxane (5 mL) and H₂O (1 mL) was degassed and purged with N₂ for 3 times. The mixture was stirred at 100° C. for 12 h, cooled to rt, poured into H₂O (5 mL) and extracted with EtOAc (20 mL×3). The combined organic layers were dried over Na₂SO₄, filtered and concentrated. The residue was purified by prep-HPLC to give tert-butyl 4-[4-[[6-(2,6-dichlorophenyl)-8-isopropenyl-5-oxo-pyrido[4,3-d]pyrimidin-2-yl]amino]pyrazol-1-yl]piperidine-1-carboxylate (IM6, 80 mg) as a yellow solid. ¹H NMR (400 MHz, DMSO-d₆) δ=10.40 (s, 1H), 9.27-9.07 (m, 1H), 8.20-8.02 (m, 1H), 7.77-7.66 (m, 3H), 7.65-7.57 (m, 2H), 5.48-5.13 (m, 2H), 4.45-4.28 (m, 1H), 4.14-3.94 (m, 2H), 3.10-2.82 (m, 2H), 2.24-2.16 (m, 3H), 2.11-1.97 (m, 2H), 1.84-1.65 (m, 2H), 1.45 (s, 9H). ESI [M+H]=596.2/598.2

### Preparation of Exemplary Compounds of the Invention

### Example 7: 8-bromo-6-(2,6-dichlorophenyl)-2-[[1-(1-methyl-4-piperidyl)pyrazol-4-yl]amino]pyrido[4,3-d]pyrimidin-5-one (P1)

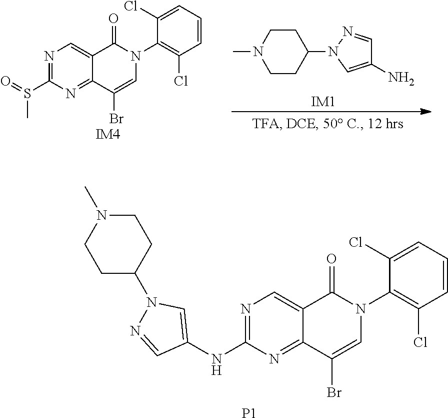

To a solution of 8-bromo-6-(2,6-dichlorophenyl)-2-methylsulfiny-pyrido[4,3-d]pyrimidin-5-one (IM4, 100 mg, 230.89 μmol) in DCE (8 mL) was added 1-(1-methyl-4-piperidyl)pyrazol-4-amine (IM1, 83.24 mg, 461.78 μmol, 2 eq.). and TFA (26.33 mg, 230.89 mol, 1 eq.). The mixture was stirred at 50° C. for 12 h, concentrated and purified by prep-HPLC to give 8-bromo-6-(2,6-dichlorophenyl)-2-[[1-(1-methyl-4-piperidyl)pyrazol-4-yl]amino]pyrido[4,3-d]pyrimidin-5-one (P1, 141.97 mg) as a yellow solid. ¹H NMR (400 MHz, DMSO-d₆) δ=10.71 (s, 1H), 9.09 (s, 1H), 8.41 (d, J=18.39 Hz, 2H), 7.82 (s, 1H), 7.71-7.76 (m, 2H), 7.56-7.66 (m, 1H), 4.21 (br s, 1H), 3.00 (br s, 2H), 2.34 (br s, 4H), 1.91-2.15 (m, 5H). ESI [M+H]=550.0/548.0.

### Example 8: 6-(2,6-dichlorophenyl)-8-methyl-2-[[1-(1-methyl-4-piperidyl)pyrazol-4-yl]amino]pyrido[4,3-d]pyrimidin-5-one (P2)

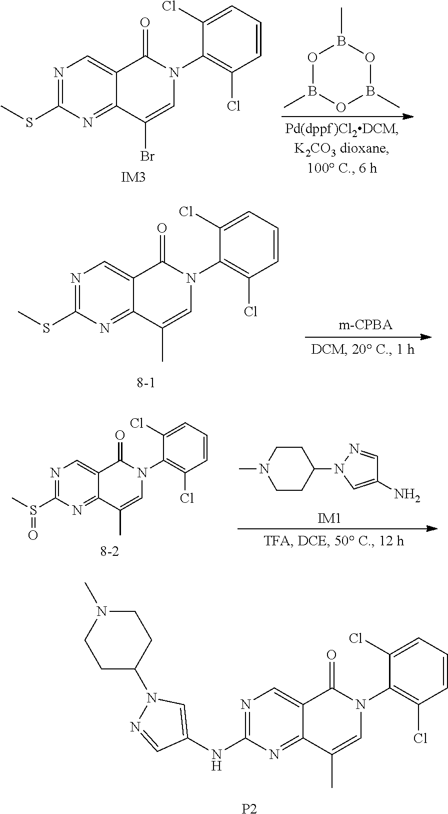

Step 1: A mixture of 8-bromo-6-(2,6-dichlorophenyl)-2-methylsulfanyl-pyrido[4,3-d]pyrimidin-5-one (IM3, 0.1 g, 239.75 μmol.), 2,4,6-trimethyl-1,3,5,2,4,6-trioxatriborinane (120.39 mg, 479.49 μmol, 50% purity, 2 eq.), Pd(dppf)Cl₂·DCM (9.79 mg, 11.99 μmol, 0.05 eq.) and K₂CO₃ (99.41 mg, 719.24 μmol, 3 eq.) in dioxane (12 mL) was stirred at 100° C. under N₂ for 6 h. The mixture was concentrated and the residue was purified by prep-HPLC to give 6-(2,6-dichlorophenyl)-8-methyl-2-methylsulfanyl-pyrido[4,3-d]pyrimidin-5-one (8-1, 30 mg) as an off-white solid. ESI [M+H]=352.0/354.0.

Step 2: To a solution of 6-(2,6-dichlorophenyl)-8-methyl-2-methylsulfanyl-pyrido[4,3-d]pyrimidin-5-one (8-1, 20 mg, 56.78 μmol, 1 eq.) in DCM (1 mL) was added m-CPBA (9.80 mg, 56.78 μmol, 85% purity, 1 eq.). The mixture was stirred at 20° C. for 1 h and then used into next step without further purification. ESI [M+H]=368.0/370.0.

Step 3: To a solution of 6-(2,6-dichlorophenyl)-8-methyl-2-methylsulfinyl-pyrido[4,3-d]pyrimidin-5-one (8-2, 9.58 mg, 26.03 μmol) in DCE (0.5 mL) was added a solution of 1-(1-methyl-4-piperidyl)pyrazol-4-amine (IM1, 9.38 mg, 52.05 μmol, 2 eq.) in DCE (1 mL), followed by addition of TFA (2.97 mg, 26.03 μmol, 1 eq.). The mixture was stirred at 50° C. for 12 h and then concentrated. The residue was purified by prep-HPLC to give 6-(2,6-dichlorophenyl)-8-methyl-2-[[1-(1-methyl-4-piperidyl)pyrazol-4-yl]amino]pyrido[4,3-d]pyrimidin-5-one (P2, 9.39 mg, HCl) as a yellow gum. ¹H NMR (400 MHz, DMSO-d₆) δ=10.17-10.48 (m, 1H), 9.47-9.90 (m, 1H), 9.11 (s, 1H), 8.12 (s, 1H), 7.79 (s, 1H), 7.73 (d, J=8.13 Hz, 2H), 7.67 (s, 1H), 7.56-7.61 (m, 1H), 4.47 (br s, 1H), 3.41 (br s, 2H), 3.16 (br d, J=10.88 Hz, 2H), 2.83 (br s, 3H), 2.07-2.33 (m, 7H). ESI [M+H]=484.1/486.1.

### Example 9: 8-cyclopropyl-6-(2,6-dichlorophenyl)-2-[[1-(4-piperidyl)pyrazol-4-yl]amino]pyrido[4,3-d]pyrimidin-5-one (P3)

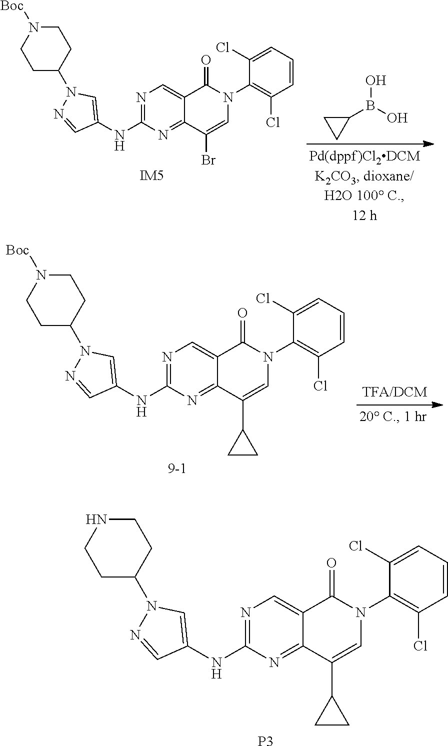

Step 1: A mixture of tert-butyl 4-[4-[[8-bromo-6-(2,6-dichlorophenyl)-5-oxo-pyrido[4,3-d]pyrimidin-2-yl]amino]pyrazol-1-yl]piperidine-1-carboxylate (IM5, 200 mg, 314.79 mol), cyclopropylboronic acid (135.20 mg, 1.57 mmol, 5 eq.), Pd(dppf)Cl₂·DCM (128.54 mg, 157.40 μmol, 0.5 eq.) and K₂CO₃ (87.01 mg, 629.59 μmol, 2 eq.) in dioxane (4 mL) and H₂O (1 mL) was degassed and purged with N₂ for 3 times. The resulting mixture was stirred at 100° C. for 12 h, cooled to rt, concentrated, diluted with H₂O (10 mL) and extracted with Ethyl acetate (10 mL×3). The combined organic layers was dried over Na₂SO₄, filtered and concentrated. The residue was purified by prep-HPLC to give tert-butyl 4-[4-[[8-cyclopropyl-6-(2,6-dichlorophenyl)-5-oxo-pyrido[4,3-d]pyrimidin-2-yl]amino]pyrazol-1-yl]piperidine-1-carboxylate (9-1, 70 mg) as a yellow solid. ESI [M+H]=596.2/598.2.

Step 2: To a solution of tert-butyl 4-[4-[[8-cyclopropyl-6-(2,6dichlorophenyl)-5-oxo-pyrido[4,3-d]pyrimidin-2-yl]amino]pyrazol-1-yl]piperidine-1-carboxylate (9-1, 70 mg, 117.35 μmol) in DCM (10 mL) was added TFA (7.70 g, 67.53 mmol) and the mixture was stirred at 20° C. for 1 h, concentrated and the residue was purified by prep-HPLC to give 8-cyclopropyl-6-(2,6-dichlorophenyl)-2-[[1-(4-piperidyl)pyrazol-4-yl]amino]pyrido[4,3-d]pyrimidin-5-one (P3, 13.81 mg, HCl) as a yellow gum. ¹H NMR (400 MHz, DMSO-d₆) δ=10.47 (s, 1H), 9.36 (br s, 2H), 9.10 (s, 1H), 8.20 (s, 1H), 7.81 (s, 1H), 7.70 (d, J=8.00 Hz, 2H), 7.50-7.60 (m, 1H), 7.46 (s, 1H), 4.50 (br s, 1H), 3.36 (br d, J=12.26 Hz, 2H), 3.06 (br s, 2H), 2.18 (br s, 5H), 0.97 (br d, J=8.13 Hz, 2H), 0.65 (br d, J=4.50 Hz, 2H). ESI [M+H]=486.2/498.2.

### Example 10: 6-(2,6-dichlorophenyl)-8-ethyl-2-[[1-(4-piperidyl)pyrazol-4-yl]amino]pyrido[4,3-d]pyrimidin-5-one (P4) and 6-(2,6-dichlorophenyl)-8-ethyl-2-((1-(1-methylpiperidin-4-yl)-1H-pyrazol-4-yl)amino)pyrido[4,3-d]pyrimidin-5(6H)-one (P5)

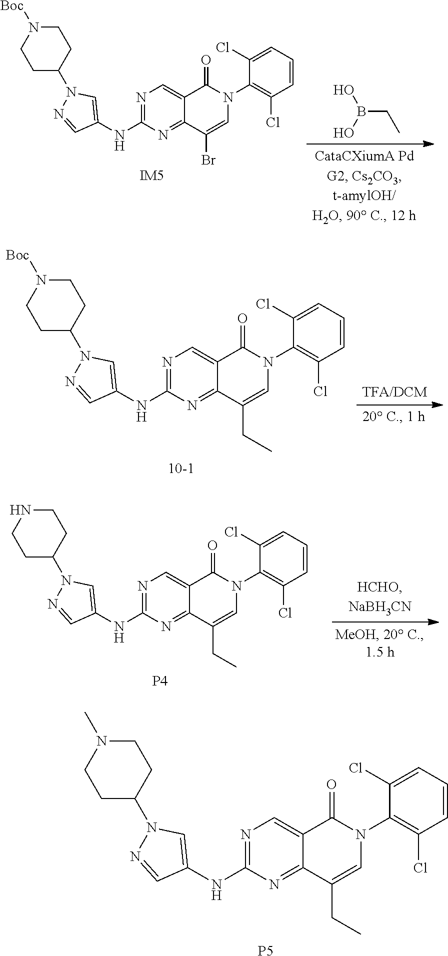

Step 1: A mixture of tert-butyl 4-[4-[[8-bromo-6-(2,6-dichlorophenyl)-5-oxo-pyrido[4,3-d]pyrimidin-2-yl]amino]pyrazol-1-yl]piperidine-1-carboxylate (IM5, 300 mg, 472.19 mol), ethylboronic acid (139.55 mg, 1.89 mmol, 4 eq.), CataCXiumA Pd G2 (63.14 mg, 94.44 mol, 0.2 eq.) and Cs₂CO₃ (307.70 mg, 944.38 μmol, 2 eq.) in 2-methyl-2-nutanol (18 mL) and H₂O (4.5 mL) was degassed and purged with N₂ for 3 times, and then the mixture was stirred at 90° C. for 12 h. The mixture was concentrated and the residue was purified by prep-HPLC to give tert-butyl 4-[4-[[6-(2,6-dichlorophenyl)-8-ethyl-5-oxo-pyrido[4,3-d]pyrimidin-2-yl]amino]pyrazol-1-yl]piperidine-1-carboxylate (10-1, 30 mg) as a yellow solid. ¹H NMR (400 MHz, DMSO-d₆) δ=10.38 (s, 1H), 9.11 (s, 1H), 8.12 (s, 1H), 7.69-7.75 (m, 3H), 7.49-7.64 (m, 2H), 4.38 (br s, 1H), 4.04 (br d, J=12.26 Hz, 2H), 2.87-3.01 (m, 2H), 2.75 (br d, J=7.38 Hz, 2H), 2.05 (br d, J=10.76 Hz, 2H), 1.70-1.80 (m, 2H), 1.42 (s, 9H), 1.26 (t, J=7.38 Hz, 3H). ESI [M+H]=584.2/586.2.

Step 2: A mixture of tert-butyl 4-[4-[[6-(2,6-dichlorophenyl)-8-ethyl-5-oxo-pyrido[4,3-d]pyrimidin-2-yl]amino]pyrazol-1-yl]piperidine-1-carboxylate (10-1, 30 mg, 51.33 mol) in TFA/DCM (1:3, 4 mL) was stirred at 20° C. for 1 h. The mixture was concentrated and the residue was purified by prep-HPLC to give 6-(2,6-dichlorophenyl)-8-ethyl-2-[[1-(4-piperidyl)pyrazol-4-yl]amino]pyrido[4,3-d]pyrimidin-5-one (P4, 23.78 mg, HCl) as a yellow solid. ¹H NMR (400 MHz, DMSO-d₆) δ=10.43 (s, 1H), 9.24-9.46 (m, 3H), 8.10 (s, 1H), 7.78 (s, 1H), 7.71 (d, J=8.13 Hz, 2H), 7.49-7.60 (m, 2H), 4.45-4.59 (m, 1H), 3.37 (br d, J=12.38 Hz, 2H), 3.07 (br s, 2H), 2.61-2.79 (m, 2H), 2.19 (br s, 4H), 1.14-1.34 (m, 3H). ESI [M+H]=484.1/486.1.

Step 3: To a solution of 6-(2,6-dichlorophenyl)-8-ethyl-2-[[1-(4-piperidyl)pyrazol-4-yl]amino]pyrido[4,3-d]pyrimidin-5-one (P4, 80 mg, 133.69 μmol) in MeOH (4 mL) was added HCHO (32.55 mg, 401.07 μmol, 37% purity, 3 eq.). The mixture was stirred at 20° C. for 0.5 h, followed by addition of NaBH₃CN (8.40 mg, 133.69 μmol, 1 eq.). The mixture was stirred at 20° C. for 1 h, then concentrated and the residue was purified by prep-HPLC to give 6-(2,6-dichlorophenyl)-8-ethyl-2-[[1-(1-methyl-4-piperidyl)pyrazol-4-yl]amino]pyrido[4,3-d]pyrimidin-5-one (P5, 7.77 mg, HCl) as a yellow solid. ¹H NMR (400 MHz, DMSO-d₆) δ=11.10 (br s, 1H), 10.39-10.48 (m, 1H), 9.11 (s, 1H), 8.01-8.22 (m, 1H), 7.65-7.86 (m, 3H), 7.47-7.64 (m, 2H), 4.46 (br s, 1H), 3.52 (br d, J=11.88 Hz, 2H), 3.16 (br d, J=10.01 Hz, 2H), 2.63-2.84 (m, 5H), 2.18-2.44 (m, 4H), 1.09-1.33 (m, 3H). ESI [M+H]=498.2/500.1.

### Example 11: 6-(2,6-dichlorophenyl)-2-((1-(piperidin-4-yl)-1H-pyrazol-4-yl)amino)-8-(prop-1-en-2-yl)pyrido[4,3-d]pyrimidin-5(6H)-one (P6)

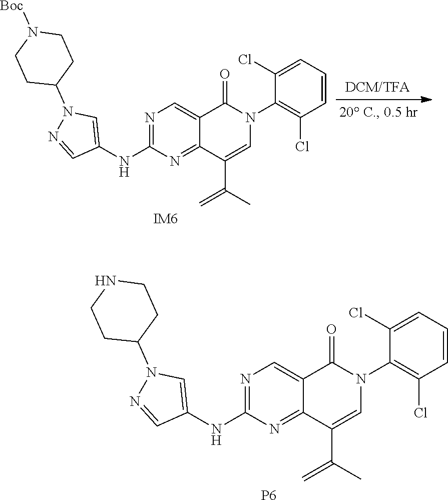

A mixture of tert-butyl 4-[4-[[6-(2,6-dichlorophenyl)-8-isopropenyl-5-oxo-pyrido[4,3-d]pyrimidin-2-yl]amino]pyrazol-1-yl]piperidine-1-carboxylate (IM6, 10 mg, 16.76 mol) in DCM (1 mL)/TFA (0.2 mL) was stirred at 20° C. for 0.5 h. The mixture was concentrated and the residue was purified by prep-HPLC to give 6-(2,6-dichlorophenyl)-8-isopropenyl-2-[[1-(4-piperidyl)pyrazol-4-yl]amino]pyrido [4,3-d]pyrimidin-5-one (P6, 8.32 mg, HCl) as a yellow gum. ¹H NMR (400 MHz, DMSO-d₆) δ=10.56-10.15 (m, 1H), 9.45-8.86 (m, 3H), 8.18-7.98 (m, 1H), 7.83-7.47 (m, 5H), 5.52-5.40 (m, 1H), 5.36-5.15 (m, 1H), 4.58-4.43 (m, 1H), 3.41 (br d, J=11.4 Hz, 2H), 3.20-3.00 (m, 2H), 2.38-2.03 (m, 7H). ESI [M+H]=496.1/498.1

### Example 12: 6-(2,6-dichlorophenyl)-8-isopropenyl-2-[[1-(1-methyl-4-piperidyl)pyrazol-4-yl]amino]pyrido[4,3-d]pyrimidin-5-one (P7)

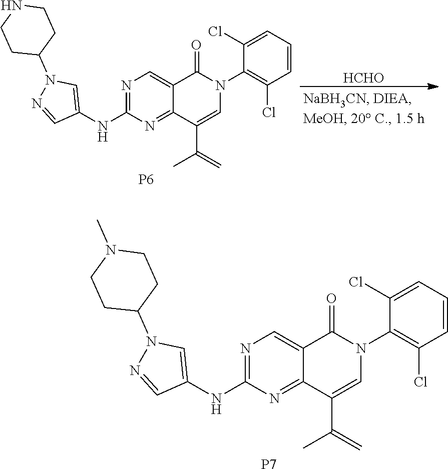

To a solution of 6-(2,6-dichlorophenyl)-8-isopropenyl-2-[[1-(4-piperidyl)pyrazol-4-yl]amino]pyrido[4,3-d]pyrimidin-5-one (P6, 70 mg, 114.68 μmol, 1 eq., TFA) in MeOH (2 mL) was added DIEA (29.64 mg, 229.35 μmol, 2 eq.) and HCHO (27.92 mg, 344.03 μmol, 37% purity, 3 eq.). The mixture was stirred at 20° C. for 0.5 h and then NaBH₃CN (7.21 mg, 114.68 μmol, 1 eq.) was added. The mixture was stirred at 20° C. for 1 h, then concentrated and the residue was purified by prep-HPLC to give 6-(2,6-dichlorophenyl)-8-isopropenyl-2-[[1-(1-methyl-4-piperidyl)pyrazol-4-yl]amino]pyrido[4,3-d]pyrimidin-5-one (P7, 13.78 mg) as a white solid. ¹H NMR (400 MHz, DMSO-d₆) δ=10.37 (s, 1H), 9.14 (s, 1H), 8.18-7.99 (m, 1H), 7.74 (d, J=8.1 Hz, 2H), 7.70-7.57 (m, 3H), 5.50-5.15 (m, 2H), 4.17-4.01 (m, 1H), 2.88 (br d, J=11.5 Hz, 2H), 2.26-2.16 (m, 6H), 2.13-1.85 (m, 6H). ESI [M+H]=510.1/512.2

### Example 13: 6-(2,6-dichlorophenyl)-8-isopropyl-2-[[1-(4-piperidyl)pyrazol-4-yl]amino]pyrido[4,3-d]pyrimidin-5-one (P8) and 6-(2,6-dichlorophenyl)-8-isopropyl-2-[[1-(1-methyl-4-piperidyl)pyrazol-4-yl]amino]pyrido[4,3-d]pyrimidin-5-one (P9)

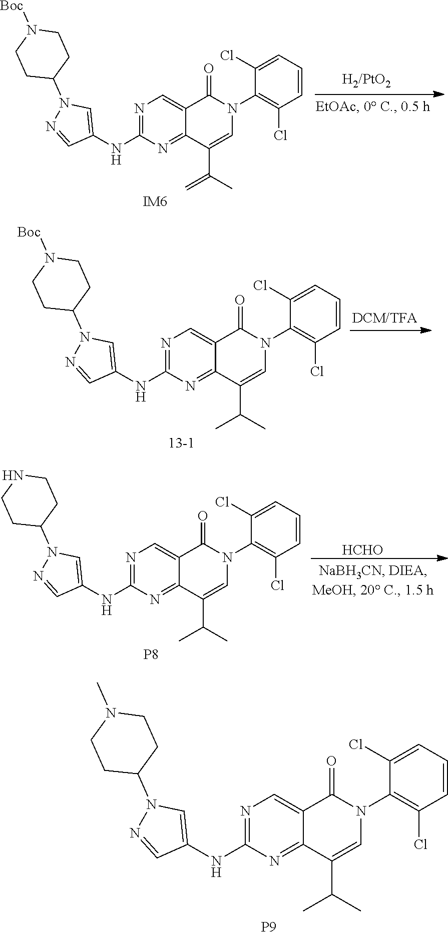

Step 1: A mixture of tert-butyl 4-[4-[[6-(2,6-dichlorophenyl)-8-isopropenyl-5-oxo-pyrido[4,3-d]pyrimidin-2-yl]amino]pyrazol-1-yl]piperidine-1-carboxylate (IM6, 10 mg, 16.76 μmol) and PtO₂ (10 mg) in EtOAc (1 mL) was degassed and purged with H₂ for 3 times and then the mixture was stirred at 0° C. for 0.5 h under H₂ (15 Psi). The mixture was filtered, concentrated and the residue was purified by prep-HPLC to give tert-butyl 4-[4-[[6-(2,6-dichlorophenyl)-8-isopropyl-5-oxo-pyrido[4,3-d]pyrimidin-2-yl]amino]pyrazol-1-yl]piperidine-1-carboxylate (13-1, 2.28 mg) was obtained as a white solid. ¹H NMR (400 MHz, METHANOL-d₄) δ=9.38-9.17 (m, 1H), 8.30-8.10 (m, 1H), 7.85-7.77 (m, 1H), 7.70-7.62 (m, 2H), 7.59-7.51 (m, 1H), 7.33-7.19 (m, 1H), 4.47-4.33 (m, 1H), 4.25 (br d, J=13.6 Hz, 2H), 3.70-3.52 (m, 1H), 3.12-2.90 (m, 2H), 2.22-2.08 (m, 2H), 2.00-1.86 (m, 2H), 1.51 (s, 9H), 1.44-1.28 (m, 6H). ESI [M+H]=598.2/600.2

Step 2: A mixture of tert-butyl 4-[4-[[6-(2,6-dichlorophenyl)-8-isopropyl-5-oxo-pyrido[4,3-d]pyrimidin-2-yl]amino]pyrazol-1-yl]piperidine-1-carboxylate (13-1, 95 mg, 158.72 μmol,) in DCM (2 mL)/TFA (0.4 mL) was stirred at 20° C. for 0.5 h. The mixture was concentrated and the residue was purified by prep-HPLC to give 6-(2,6-dichlorophenyl)-8-isopropyl-2-[[1-(4-piperidyl)pyrazol-4-yl]amino]pyrido[4,3-d]pyrimidin-5-one (P8, 50 mg, 81.64 mol, TFA) as a gray solid. ¹H NMR (400 MHz, DMSO-d₆) δ=10.47-10.15 (m, 1H), 9.26-9.09 (m, 1H), 8.85-8.37 (m, 2H), 8.15-8.01 (m, 1H), 7.82 (s, 1H), 7.74 (d, J=8.2 Hz, 2H), 7.71-7.57 (m, 1H), 7.52-7.45 (m, 1H), 4.59-4.46 (m, 1H), 3.54-3.44 (m, 3H), 3.23-3.02 (m, 2H), 2.27-2.05 (m, 4H), 1.43-0.92 (m, 6H). ESI [M+H]=498.1/500.2

Step 3: To a solution of 6-(2,6-dichlorophenyl)-8-isopropyl-2-[[1-(4-piperidyl)pyrazol-4-yl]amino]pyrido[4,3-d]pyrimidin-5-one (P8, 35 mg, 57.15 μmol, TFA) in MeOH (2 mL) was added DIEA (14.77 mg, 114.30 μmol, 2 eq.) and HCHO (13.91 mg, 171.45 mol, 37% purity, 3 eq.). The mixture was stirred at 20° C. for 0.5 h and then NaBH₃CN (3.59 mg, 57.15 μmol, 1 eq.) was added. The mixture was stirred at 20° C. for 1 h, then concentrated and the residue was purified by prep-HPLC to give 6-(2,6-dichlorophenyl)-8-isopropyl-2-[[1-(1-methyl-4-piperidyl)pyrazol-4-yl]amino]pyrido[4,3-d]pyrimidin-5-one (P9, 11.8 mg) as an off-white solid. ¹H NMR (400 MHz, DMSO-d₆) δ=10.46-10.09 (m, 1H), 9.27-9.05 (m, 1H), 8.17-7.92 (m, 1H), 7.73 (br d, J=8.4 Hz, 2H), 7.59 (br d, J=7.1 Hz, 1H), 7.52-7.41 (m, 1H), 4.24-4.06 (m, 1H), 3.30-3.11 (m, 3H), 3.02-2.86 (m, 2H), 2.31-2.13 (m, 4H), 2.09-1.92 (m, 3H), 1.51-0.83 (m, 6H). ESI [M+H]=512.2/514.2

### Example 14: 8-bromo-6-(2,6-dichlorophenyl)-2-(1,2,3,4-tetrahydroisoquinolin-7-ylamino)pyrido[4,3-d]pyrimidin-5-one (P10) and 8-bromo-6-(2,6-dichlorophenyl)-2-[(2-methyl-3,4-dihydro-1H-isoquinolin-7-yl)amino]pyrido[4,3-d]pyrimidin-5-one (P11)

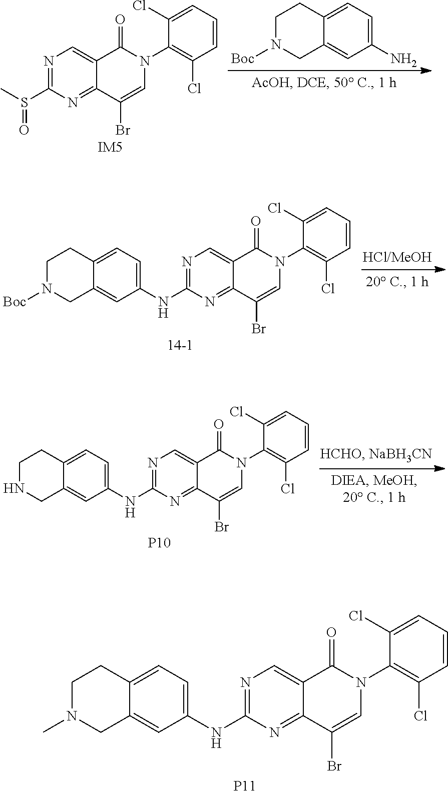

Step 1: To a solution of 8-bromo-6-(2,6-dichlorophenyl)-2-methylsulfinyl-pyrido[4,3-d]pyrimidin-5-one (IM5, 180 mg, 415.60 μmol) in DCE (2 mL) was added tert-butyl 7-amino-3,4-dihydro-1H-isoquinoline-2-carboxylate (206.40 mg, 831.20 μmol, 2.0 eq.), followed by addition of AcOH (124.78 mg, 2.08 mmol, 5.0 eq.). The mixture was stirred at 50° C. for 1 h, then concentrated to give tert-butyl 7-[8-bromo-6-(2,6-dichlorophenyl)-5-oxo-pyrido[4,3-d]pyrimidin-2-yl]amino]-3,4-dihydro-1H-isoquinoline-2-carboxylate (14-1, 250 mg, crude) as a yellow solid. ESI [M+H]=618.2/616.2.

Step 2: A mixture of tert-butyl 7-[[8-bromo-6-(2,6-dichlorophenyl)-5-oxo-pyrido[4,3-d]pyrimidin-2-yl]amino]-3,4-dihydro-1H-isoquinoline-2-carboxylate (14-1, 250 mg, 404.98 μmol) in HCl/MeOH (3 mL, 4 M) was stirred at 20° C. for 1 h. The mixture was concentrated and the residue was purified by prep-HPLC to give 8-bromo-6-(2,6-dichlorophenyl)-2-(1,2,3,4-tetrahydroisoquinolin-7-ylamino)pyrido[4,3-d]pyrimidin-5-one (P10, 148.05 mg, HCl) as a yellow solid. ¹H NMR (400 MHz, DMSO-d₆) δ=10.71 (br s, 1H), 9.37 (br s, 2H), 9.20 (s, 1H), 8.47 (s, 1H), 8.17 (br s, 1H), 7.76 (d, J=8.00 Hz, 3H), 7.58-7.66 (m, 1H), 7.24 (d, J=8.38 Hz, 1H), 4.61 (br s, 2H), 4.30 (br s, 2H), 3.00 (br t, J=5.88 Hz, 2H). ESI [M+H]=518.0/516.0

Step 3: To a solution of 8-bromo-6-(2,6-dichlorophenyl)-2-(1,2,3,4-tetrahydroisoquinolin-7-yl)amino)pyrido[4,3-d]pyrimidin-5-one (P10, 60 mg, 108.37 μmol, HCl) in MeOH (2 mL) was added DIEA (28.01 mg, 216.74 μmol, 2.0 eq.) and HCHO (26.39 mg, 325.11 μmol, 37% purity, 3.0 eq.). The mixture was stirred at 20° C. for 0.5 h and then NaBH₃CN (6.81 mg, 108.37 μmol, 1.0 eq.) was added. The mixture was stirred at 20° C. for 0.5 h, concentrated and the residue was purified by prep-HPLC to give 8-bromo-6-(2,6-dichlorophenyl)-2-[(2-methyl-3,4-dihydro-1H-isoquinolin-7-yl)amino]pyrido[4,3-d]pyrimidin-5-one (P11, 26.22 mg, HCl) as a yellow solid. ¹H NMR (400 MHz, DMSO-d₆) δ=11.58 (br s, 1H), 10.78 (br s, 1H), 9.24 (s, 1H), 8.22-8.56 (m, 2H), 7.79 (d, J=8.13 Hz, 3H), 7.61-7.70 (m, 1H), 7.28 (d, J=8.50 Hz, 1H), 4.31-4.58 (m, 2H), 3.67 (br d, J=8.76 Hz, 1H), 3.24-3.44 (m, 2H), 2.90-3.08 (m, 4H). ESI [M+H]=532.0/530.0

### Example 15: Preparation of Compound P30

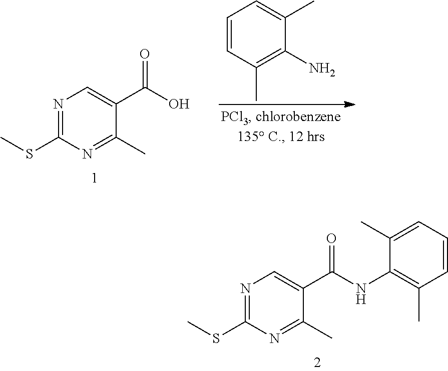

### (i) Preparation of Compound 2

A mixture of 4-methyl-2-methylsulfanyl-pyrimidine-5-carboxylic acid (3 g, 16.29 mmol, 1.0 eq.), 2,6-dimethylaniline (1.97 g, 16.29 mmol, 1.0 eq.) in chlorobenzene (50 mL) was degassed and purged with N₂ for 3 times, following by the addition of PCl₃ (2.24 g, 16.29 mmol, 1.0 eq.). The mixture was stirred at 135° C. for 3 hrs under N₂ atmosphere. The mixture was concentrated and the residue was poured into sat.aq. Na₂CO₃ (50 mL), extracted with 2-methyltetrahydrofuran (50 mL×3). The organic phase was dried over Na₂SO₄ and concentrated. The residue was triturated with EtOH (20 mL) to give N-(2,6-dimethylphenyl)-4-methyl-2-methylsulfanyl-pyrimidine-5-carboxamide (2.45 g, 8.53 mmol, 52.35% yield) as a yellow solid. ESI [M+H]=288.1.

### (ii) Preparation of Compound 3

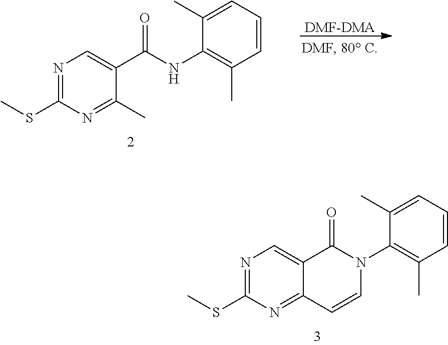

To a solution of N-(2,6-dimethylphenyl)-4-methyl-2-methylsulfanyl-pyrimidine-5-carboxamide (2.45 g, 8.53 mmol, 1.0 eq.) in DMF (50 mL) was added DMF-DMA (6.10 g, 51.15 mmol, 6.0 eq.) and the mixture was stirred at 80° C. for 12 hrs. The mixture was concentrated and the residue was triturated with EtOH (15 mL) to give 6-(2,6-dimethylphenyl)-2-methylsulfanyl-pyrido[4,3-d]pyrimidin-5-one (1.76 g, 5.92 mmol, 69.42% yield) as a pale yellow solid. ESI [M+H]=298.1.

(iii) Preparation of Compound 4

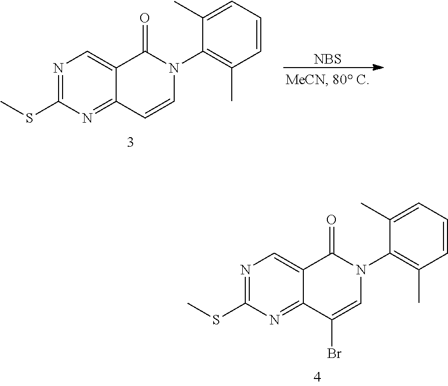

To a solution of 6-(2,6-dimethylphenyl)-2-methylsulfanyl-pyrido[4,3-d]pyrimidin-5-one (1.76 g, 5.92 mmol, 1.0 eq.) in ACN (30 mL) was added NBS (1.58 g, 8.88 mmol, 1.5 eq.) and the mixture was degassed and purged with N₂ for 3 times. The mixture was stirred at 80° C. for 12 hrs under N₂ atmosphere. The mixture was concentrated and the residue was triturated with EtOH (10 mL) to give 8-bromo-6-(2,6-dimethylphenyl)-2-methylsulfanyl-pyrido[4,3-d]pyrimidin-5-one (2 g, 5.32 mmol, 89.81% yield) as a pale yellow solid. ESI [M+H]=376.2/378.2.

### (iv) Preparation of Compound 5

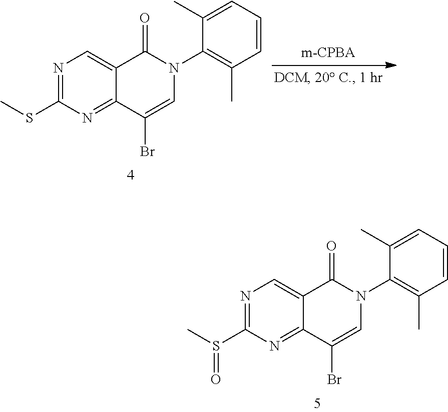 [SMILES: CSc1ncc2c(=O)n(-c3c(C)cccc3C)cc(Br)c2n1 ; Cc1cccc(C)c1-n1cc(Br)c2nc(S(C)=O)ncc2c1=O]

To a solution of 8-bromo-6-(2,6-dimethylphenyl)-2-methylsulfanyl-pyrido[4,3-d] pyrimidin-5-one (500 mg, 1.33 mmol, 1.0 eq.) in DCM (10 mL) was added m-CPBA (404.67 mg, 1.99 mmol, 85% purity, 1.5 eq) at 0° C. The mixture was stirred at 20° C. for 1 hr and then used into next step without further purification. ESI [M+H]=391.9/393.9.

### (v) Preparation of Compound 6

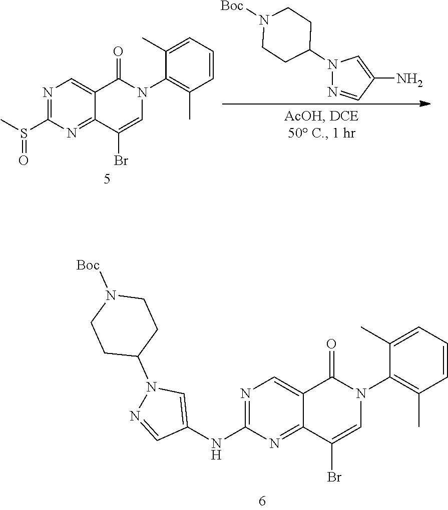 [SMILES: Cc1cccc(C)c1-n1cc(Br)c2nc(S(C)=O)ncc2c1=O ; CC(C)(C)OC(=O)N1CCC(n2cc(N)cn2)CC1 ; Cc1cccc(C)c1-n1cc(Br)c2nc(Nc3cnn(C4CCN(C(=O)OC(C)(C)C)CC4)c3)ncc2c1=O]

To a solution of 8-bromo-6-(2,6-dimethylphenyl)-2-methylsulfinyl-pyrido[4,3-d]pyrimidin-5-one (520 mg, 1.33 mmol, 1.0 eq.) in DCE (12 mL) was added tert-butyl 4-(4-aminopyrazol-1-yl)piperidine-1-carboxylate (708.46 mg, 2.66 mmol, 2.0 eq.) and AcOH (2.00 g, 33.25 mmol, 25.0 eq.). The mixture was stirred at 50° C. for 1 hr, then concentrated to give tert-butyl 4-[4-[[8-bromo-6-(2,6-dimethylphenyl)-5-oxo-pyrido[4,3-d]pyrimidin-2-yl]amino]pyrazol-1-yl]piperidine-1-carboxylate (710 mg, crude) as a brown gum. ESI [M+H-56]=538.3/540.3.

### (vi) Preparation of Compound P30

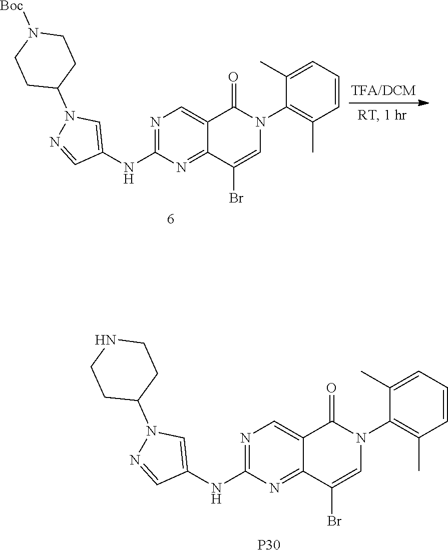

A mixture of tert-butyl 4-[4-[[8-bromo-6-(2,6-dimethylphenyl)-5-oxo-pyrido[4,3-d]pyrimidin-2-yl]amino]pyrazol-1-yl]piperidine-1-carboxylate (700 mg, 1.18 mmol, 1.0 eq.) in DCM (6 mL) and TFA (2 mL) was stirred at 20° C. for 12 hrs. The mixture was concentrated and purified by prep-HPLC (column: Phenomenex luna C18 250*50 mm*10 μm; mobile phase: [water(0.1% TFA)-ACN]; B %: 15%-45%, 10 min) to give 8-bromo-6-(2,6-dimethylphenyl)-2-[[1-(4-piperidyl)pyrazol-4-yl]amino]pyrido[4,3-d]pyrimidin-5-one (400 mg, 655.60 μmol, 55.68% yield, 99.718% purity, TFA) as a yellow solid. ¹H NMR (400 MHz, DMSO-d₆) δ=10.68 (s, 1H), 9.13 (s, 1H), 8.88-8.70 (m, 1H), 8.62-8.44 (m, 2H), 8.29-7.98 (m, 1H), 7.89-7.66 (m, 1H), 7.37-7.23 (m, 3H), 4.62-4.48 (m, 1H), 3.52-3.39 (m, 2H), 3.20-3.04 (m, 2H), 2.30-2.20 (m, 2H), 2.20-2.03 (m, 8H). ESI [M+H]=494.1/496.1.

### Example 16: Preparation of Compound P27

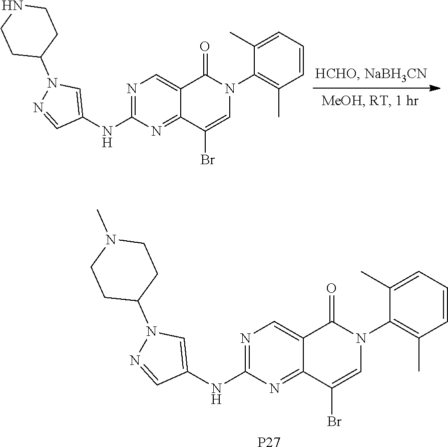

To a solution of 8-bromo-6-(2,6-dimethylphenyl)-2-[[1-(4-piperidyl)pyrazol-4-yl]amino]pyrido[4,3-d]pyrimidin-5-one (250 mg, 505.68 μmol, 1.0 eq., TFA) in MeOH (4 mL) was added DIEA (130.71 mg, 1.01 mmol, 2.0 eq.) and HCHO (123.13 mg, 1.52 mmol, 37% purity, 3.0 eq.). The mixture was stirred at 20° C. for 0.5 hr and followed by the addition of NaBH₃CN (63.56 mg, 1.01 mmol, 2.0 eq.). The mixture was stirred at 20° C. for 1 hr, then concentrated and purified by prep-HPLC (column: Welch Xtimate C18 100*25 mm*3 um; mobile phase: [water(0.05% HCl)-ACN]; B %: 10%-30%, 8 min) to give 8-bromo-6-(2,6-dimethylphenyl)-2-[[1-(1-methyl-4-piperidyl)pyrazol-4-yl]amino]pyrido[4,3-d]pyrimidin-5-one (144.15 mg, 258.29 μmol, 51.08% yield, 97.630% purity, HCl) as a yellow solid. ¹H NMR (400 MHz, DMSO-d₆) δ 11.06-10.78 (m, 1H), 10.72-10.43 (m, 1H), 9.10 (s, 1H), 8.60-8.42 (m, 1H), 8.22-7.91 (m, 1H), 7.63 (d, J=1.5 Hz, 1H), 7.34-7.20 (m, 3H), 4.54-4.40 (m, 1H), 3.58-3.33 (m, 2H), 3.23-3.07 (m, 2H), 2.75 (br d, J=4.6 Hz, 3H), 2.40-2.19 (m, 4H), 2.08-1.99 (m, 6H). ESI [M+H]=508.1/510.1.

### Example 17: Preparation of Compound P24

### (i) Preparation of Compound 2

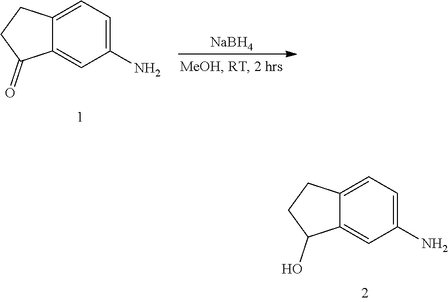 [SMILES: Nc1ccc2c(c1)C(=O)CC2 ; "NaBH4" ; Nc1ccc2c(c1)C(O)CC2]

To a solution of 6-aminoindan-1-one (250 mg, 1.70 mmol, 1.0 eq.) in MeOH (4 mL) was added NaBH₄ (0.02 g, 528.65 μmol, 0.3 eq.) and stirred at 20° C. for 2 hrs. The mixture was quenched by 1 N HCl (5 mL) followed by the addition of sat.aq.Na₂CO₃ (10 mL) and extracted with 2-methyltetrahydrofuran (20 mL×3), dried over Na₂SO₄. The organic layers were filtered and concentrated to give 6-aminoindan-1-ol (240 mg, crude) as a pink solid. ¹H NMR (400 MHz, DMSO-d₆) δ=6.90-6.83 (m, 1H), 6.58 (d, J=1.7 Hz, 1H), 6.44 (dd, J=2.1, 7.9 Hz, 1H), 5.04 (d, J=6.4 Hz, 1H), 4.94 (br s, 2H), 4.91-4.87 (m, 1H), 2.79-2.68 (m, 1H), 2.59-2.53 (m, 1H), 2.31-2.19 (m, 1H), 1.77-1.65 (m, 1H). ESI [M+H]=150.3.

### (ii) Preparation of Compound 4

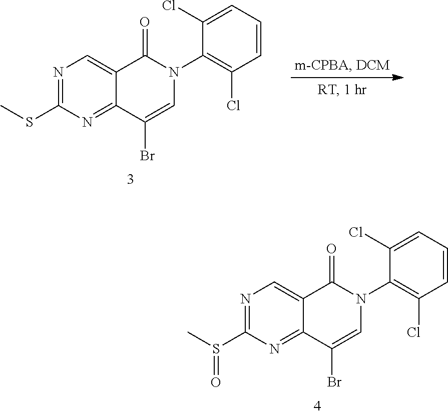 [SMILES: CSc1ncc2c(=O)n(-c3c(Cl)cccc3Cl)cc(Br)c2n1 ; CS(=O)c1ncc2c(=O)n(-c3c(Cl)cccc3Cl)cc(Br)c2n1]

To a solution of 8-bromo-6-(2,6-dichlorophenyl)-2-methylsulfanyl-pyrido[4,3-d]pyrimidin-5-one (100 mg, 239.75 μmol, 1.0 eq.) in DCM (2 mL) was added m-CPBA (63.28 mg, 311.67 μmol, 85% purity, 1.3 eq.) at 0° C. The mixture was stirred at 20° C. for 1 hr and then used into next step without further purification. ESI [M+H]=431.8/433.8.

(iii) Preparation of Compound P24

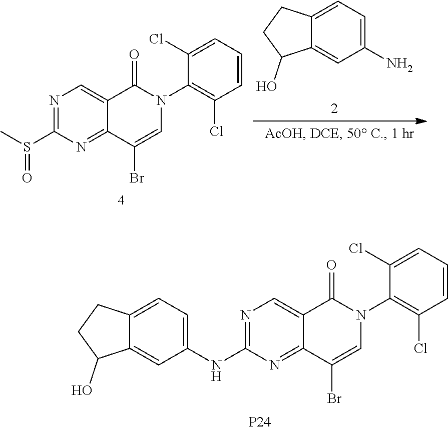

To a solution of 8-bromo-6-(2,6-dichlorophenyl)-2-methylsulfinyl-pyrido[4,3-d]pyrimidin-5-one (100 mg, 230.89 μmol, 1.0 eq.) in DCE (3 mL) was added 6-aminoindan-1-ol (68.89 mg, 461.78 μmol, 2.0 eq.) and AcOH (525.00 mg, 8.74 mmol, 0.5 mL, 37.87 eq.). The mixture was stirred at 50° C. for 1 hr. The mixture was concentrated and the residue was purified by prep-HPLC (column: Phenomenex luna C18 100*40 mm*3 μm; mobile phase: [water(0.04% HCl)-ACN]; B %: 30%-60%, 7 min) to give 8-bromo-6-(2,6-dichlorophenyl)-2-[(3-hydroxyindan-5-yl)amino]pyrido[4,3-d]pyrimidin-5-one (90 mg, 166.17 μmol, 71.97% yield, 95.673% purity) as a yellow solid. ¹H NMR (400 MHz, DMSO-d₆) δ 10.73-10.49 (m, 1H), 9.18 (d, J=1.0 Hz, 1H), 8.43 (s, 1H), 8.16-7.87 (m, 2H), 7.76 (d, J=8.3 Hz, 2H), 7.67-7.58 (m, 1H), 7.22 (br d, J=8.3 Hz, 1H), 5.24 (br d, J=5.9 Hz, 1H), 5.07 (br d, J=5.9 Hz, 1H), 2.98-2.83 (m, 1H), 2.76-2.63 (m, 1H), 2.42-2.32 (m, 1H), 1.89-1.75 (m, 1H). ESI [M+H]=517.0/518.9.

### Example 18: Preparation of Compound P23

### (i) Preparation of Compound 2

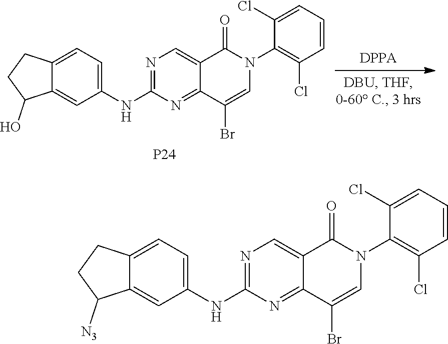

To a solution of 8-bromo-6-(2,6-dichlorophenyl)-2-[(3-hydroxyindan-5-yl)amino]pyrido[4,3-d]pyrimidin-5-one (50 mg, 96.49 μmol, 1.0 eq.) in THF (4 mL) was added DPPA (53.11 mg, 192.98 μmol, 2.0 eq.), followed by addition of DBU (44.07 mg, 289.47 μmol, 3.0 eq.) at 0° C. under N₂ atmosphere. The mixture was stirred at 60° C. for 3 hrs under N₂ atmosphere. The reaction mixture was added H₂O (15 mL) and extracted with ethyl acetate (10 mL×3), dried over Na₂SO₄. The organic layers were filtered and concentrated to give 2-[(3-azidoindan-5-yl)amino]-8-bromo-6-(2,6-dichlorophenyl)pyrido[4,3-d]pyrimidin-5-one (60 mg, crude) as a yellow gum. ESI [M+H]=542.1/544.1.

### (ii) Preparation of Compound P23

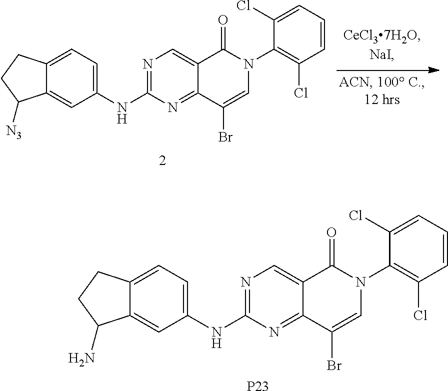

To a solution of 2-[(3-azidoindan-5-yl)amino]-8-bromo-6-(2,6-dichlorophenyl)pyrido[4,3-d]pyrimidin-5-one (30 mg, 55.23 μmol, 1.0 eq.) in ACN (5 mL) was added CeCl₃·7H₂O (41.15 mg, 110.46 μmol, 2.0 eq.) and NaI (74.51 mg, 497.05 μmol, 9.0 eq.). The mixture was stirred at 100° C. for 12 hrs and then concentrated. The residue was purified by prep-HPLC (column: mobile phase: [water(0.04% HCl)-ACN]; B %: 25%-45%, 7 min) to give 2-[(3-aminoindan-5-yl)amino]-8-bromo-6-(2,6-dichlorophenyl)pyrido[4,3-d]pyrimidin-5-one (11.65 mg, 20.72 μmol, 37.52% yield, 98.485% purity, HCl) as a dark yellow solid. ¹H NMR (400 MHz, DMSO-d₆) δ 10.83-10.60 (m, 1H), 9.19 (s, 1H), 8.57 (br s, 3H), 8.43 (s, 1H), 8.03 (s, 1H), 7.76 (d, J=8.1 Hz, 2H), 7.68-7.57 (m, 1H), 7.38-7.20 (m, 2H), 4.71 (br s, 1H), 3.16-3.02 (m, 1H), 2.94-2.80 (m, 1H), 2.50-2.44 (m, 1H), 2.17-2.02 (m, 1H). ESI [M+H]=516/517.9.

### Example 19: Preparation of Compound P22

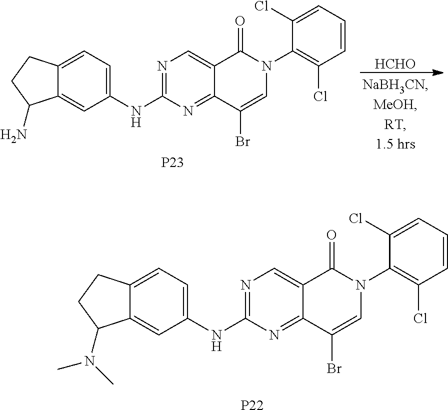

To a solution of 2-[(3-aminoindan-5-yl)amino]-8-bromo-6-(2,6-dichlorophenyl) pyrido[4,3-d]pyrimidin-5-one (290 mg, 560.71 μmol, 1.0 eq.) in MeOH (5 mL) was added DIEA (144.93 mg, 1.12 mmol, 2.0 eq.) and HCHO (136.52 mg, 1.68 mmol, 37% purity, 3.0 eq.). The mixture was stirred at 20° C. for 0.5 hr and followed by addition of NaBH₃CN (70.4 mg, 1.12 mmol, 2.0 eq.). The mixture was stirred at 20° C. for 1 hr. The mixture was concentrated and the residue was purified by prep-HPLC (column: Welch Xtimate C18 100*25 mm*3 μm; mobile phase: [water(0.04% HCl)-ACN];B %: 20%-40%, 8 min) to give 8-bromo-6-(2,6-dichlorophenyl)-2-[[3-(dimethylamino)indan-5-yl]amino]pyrido[4,3-d]pyrimidin-5-one (13.01 mg, 21.69 μmol, 3.87% yield, 96.970% purity, HCl) as a yellow solid. ¹H NMR (400 MHz, DMSO-d₆) δ 10.92-10.62 (m, 2H), 9.20 (s, 1H), 8.49-8.42 (m, 1H), 8.06-7.85 (m, 1H), 7.76 (d, J=8.1 Hz, 2H), 7.65-7.58 (m, 1H), 7.41-7.32 (m, 1H), 7.21-7.10 (m, 1H), 4.99-4.90 (m, 1H), 3.18-3.06 (m, 1H), 2.93-2.83 (m, 1H), 2.75 (br s, 3H), 2.63 (br d, J=3.4 Hz, 3H), 2.47-2.40 (m, 2H). ESI [M+H]=544.0/546.0.

### Example 20: Preparation of Compound P21

### (i) Preparation of Compound 2

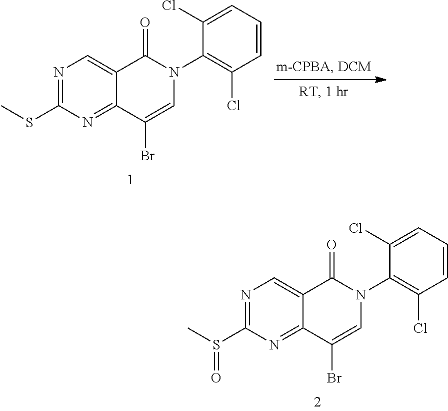 [SMILES: CSc1ncc2c(=O)n(-c3c(Cl)cccc3Cl)cc(Br)c2n1 ; CS(=O)c1ncc2c(=O)n(-c3c(Cl)cccc3Cl)cc(Br)c2n1]

To a solution of 8-bromo-6-(2,6-dichlorophenyl)-2-methylsulfanyl-pyrido[4,3-d]pyrimidin-5-one (200 mg, 479.49 μmol, 1.0 eq.) in DCM (5 mL) was added m-CPBA (146.02 mg, 719.24 μmol, 85% purity, 1.5 eq.) at 0° C. The mixture was stirred at 20° C. for 1 hr and then used for next step without further purification. ESI [M+H]=431.8/433.8.

### (ii) Preparation of Compound P21

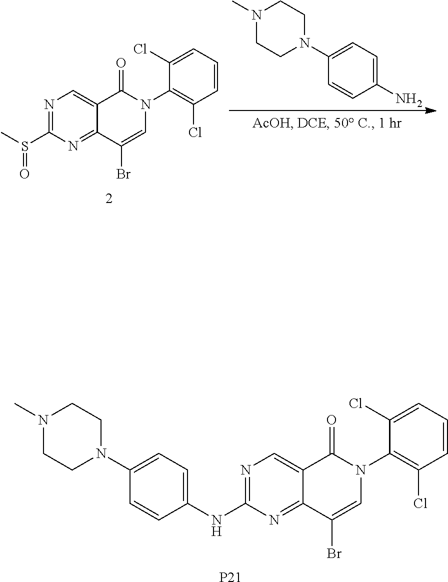

To a solution of 8-bromo-6-(2,6-dichlorophenyl)-2-methylsulfinyl-pyrido[4,3-d]pyrimidin-5-one (200 mg, 461.78 μmol, 1.0 eq.) in DCE (5 mL) was added 4-(4-methylpiperazin-1-yl)aniline (176.65 mg, 923.56 μmol, 2.0 eq.) and AcOH (138.65 mg, 2.31 mmol, 5.0 eq.). The mixture was stirred at 50° C. for 1 hr. The mixture was concentrated and the residue was purified by prep-HPLC (HCl condition, column: Phenomenex Luna C18 75*30 mm*3 μm; mobile phase: [water(0.04% HCl)-ACN]; B %: 20%-50%, 8 min) to give 8-bromo-6-(2,6-dichlorophenyl)-2-[4-(4-methylpiperazin-1-yl)anilino]pyrido[4,3-d]pyrimidin-5-one (194.04 mg, 323.22 μmol, 69.99% yield, 99.40% purity, HCl) as a yellow solid. ¹H NMR (400 MHz, DMSO-d₆) δ 10.43-10.58 (m, 2H), 9.13-9.14 (m, 1H), 9.14 (s, 1H), 8.42 (s, 1H), 8.01 (br s, 2H), 7.75 (d, J=8.13 Hz, 2H), 7.61 (dd, J=7.69, 8.69 Hz, 1H), 7.05 (br d, J=8.75 Hz, 2H), 3.81 (br d, J=12.76 Hz, 2H), 3.50 (br d, J=11.63 Hz, 2H), 3.12-3.22 (m, 2H), 3.01-3.10 (m, 2H), 2.83 (d, J=4.50 Hz, 3H). ESI [M+H]=559.0/560.9.

### Example 21: Preparation of Compound P19

### (i) Preparation of Compound 2

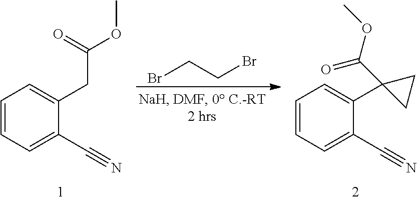

To a solution of NaH (913.33 mg, 22.83 mmol, 60% purity, 2.0 eq.) in DMF (30 mL) was added dropwise methyl 2-(2-cyanophenyl)acetate (2 g, 11.42 mmol, 1.0 eq.) at 0° C. The mixture was stirred at 0° C. for 0.5 hr, and then 1,2-dibromoethane (4.29 g, 22.83 mmol, 2.0 eq.) was added dropwise at 0° C. The resulting mixture was stirred at 20° C. for 0.5 hr and then quenched with sat.aq.NH₄Cl (80 mL). The reaction mixture was concentrated to remove the organic phase and then was diluted with H₂O (30 mL) and extracted with Ethyl acetate (40 mL×3), and then washed with brine (30 mL). The combined organic layers was dried over Na₂SO₄, filtered and concentrated to give methyl 1-(2-cyanophenyl)cyclopropanecarboxylate (2.1 g, crude) as a dark yellow oil. ¹H NMR (400 MHz, DMSO-d₆) δ 7.79-7.90 (m, 1H), 7.62-7.76 (m, 1H), 7.45-7.57 (m, 2H), 3.54-3.64 (m, 3H), 1.57-1.71 (m, 2H), 1.27-1.43 (m, 2H). ESI [M+H]=202.1.

### (ii) Preparation of Compound 3.

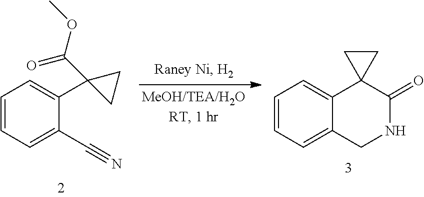

To a solution of methyl 1-(2-cyanophenyl)cyclopropanecarboxylate (2 g, 9.94 mmol, 1.0 eq.) in MeOH (60 mL)/TEA (6 mL)/H₂O (7 mL) was added Raney-Ni (2.4 g, 9.94 mmol, 1.0 eq.) under H₂ atmosphere. The suspension was degassed and purged with H₂ for 3 times. The mixture was stirred under H₂ (15 Psi) atmosphere at 20° C. for 12 hrs. The reaction mixture was filtered and the filtrate was concentrated to give spiro[1,2-dihydroisoquinoline-4,1′-cyclopropane]-3-one (1.6 g, crude) as a yellow solid. ESI [M+H]=174.2.

(iii) Preparation of Compound 4

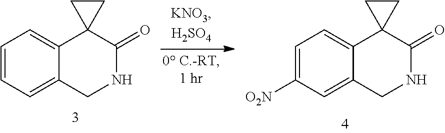 [SMILES: O=C1NCc2ccccc2C12CC2 ; O=C1NCc2cc([N+](=O)[O-])ccc2C12CC2]

To a solution of spiro[1,2-dihydroisoquinoline-4,1′-cyclopropane]-3-one (600 mg, 3.46 mmol, 1.0 eq.) in H₂SO₄ (10 mL) was added KNO₃ (420.25 mg, 4.16 mmol, 1.2 eq.) at 0° C. The mixture was stirred at 0° C. for 15 mins and then stirred at 20° C. for 45 mins. The reaction solution was poured into ice water (20 mL) and filtered to give 7-nitrospiro[1,2-dihydroisoquinoline-4,1′-cyclopropane]-3-one (670 mg, crude) as a light yellow solid. ESI [M+H]=219.1.

### (iv) Preparation of Compound 5

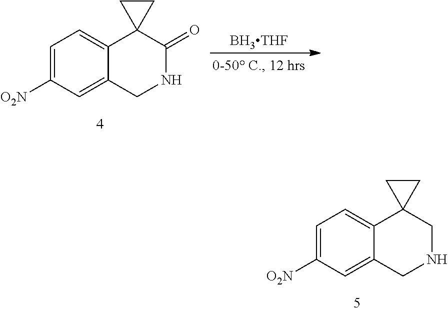 [SMILES: O=C1NCc2cc([N+](=O)[O-])ccc2C12CC2 ; O=[N+]([O-])c1ccc2c(c1)CNCC21CC1 ; "BH3·THF"]

7-nitrospiro[1,2-dihydroisoquinoline-4,1′-cyclopropane]-3-one (670 mg, 3.07 mmol, 1 eq.) was added BH₃·THF (1 M, 20 mL) at 0° C. The mixture was stirred at 50° C. for 12 hrs. The reaction mixture was quenched with 4 N HCl (5 mL) at 0° C. and stirred at 50° C. for 1 hr. The reaction mixture was adjusted to pH=8 with sat.aq.Na₂CO₃ to give 7-nitrospiro[2,3-dihydro-1H-isoquinoline-4,1′-cyclopropane] (600 mg, crude) as a yellow oil. ESI [M+H]=205.2.

### (v) Preparation of Compound 6

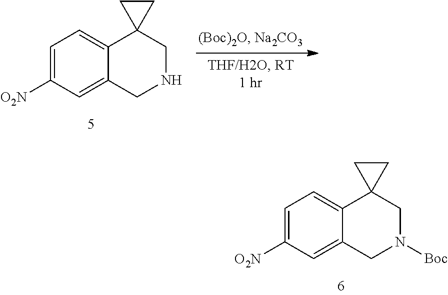 [SMILES: O=[N+]([O-])c1ccc2c(c1)CNCC21CC1 ; CC(C)(C)OC(=O)N1Cc2cc([N+](=O)[O-])ccc2C2(CC2)C1]

To a solution of 7-nitrospiro[2,3-dihydro-1H-isoquinoline-4,1′-cyclopropane](600 mg, 2.94 mmol, 1.0 eq.) in THE (8 mL) and H₂O (8 mL) was added tert-butoxycarbonyl tert-butyl carbonate (769.43 mg, 3.53 mmol, 1.2 eq.). The mixture was stirred at 20° C. for 1 hr. The reaction mixture was concentrated to remove solvent, and extracted with ethyl acetate (20 mL×3). The combined organic layers were dried over Na₂SO₄, filtered and concentrated to give a residue. The residue was purified through silica gel column chromatography (SiO2, petroleum ether: ethyl acetate=1:0 to 5:1) to give tert-butyl 7-nitrospiro[1,3-dihydroisoquinoline-4,1′-cyclopropane]-2-carboxylate as a yellow solid. ESI [M+H-tBu]=249.1.

### (vi) Preparation of Compound 7

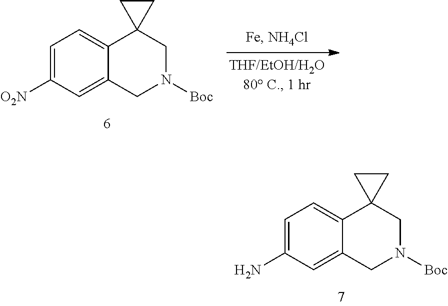 [SMILES: CC(C)(C)OC(=O)N1Cc2cc([N+](=O)[O-])ccc2C2(CC2)C1 ; CC(C)(C)OC(=O)N1Cc2cc(N)ccc2C2(CC2)C1]

To a solution of tert-butyl 7-nitrospiro[1,3-dihydroisoquinoline-4,1′-cyclopropane]-2-carboxylate (470 mg, 1.54 mmol, 1.0 eq.) in THE (3 mL), EtOH (3 mL) and H₂O (1 mL) was added Fe (431.25 mg, 7.72 mmol, 5.0 eq.) and NH₄Cl (413.03 mg, 7.72 mmol, 5.0 eq.). The mixture was stirred at 80° C. for 1 hr. The mixture was filtered and concentrated to give tert-butyl 7-aminospiro[1,3-dihydroisoquinoline-4,1′-cyclopropane]-2-carboxylate (400 mg, crude) as a yellow gum. ¹H NMR (400 MHz, DMSO-d₆) δ 7.40 (br s, 1H), 7.28 (br s, 1H), 7.15 (br s, 1H), 6.35-6.52 (m, 2H), 4.46 (br s, 2H), 3.35-3.39 (m, 2H), 1.40 (br s, 9H), 0.79 (s, 4H). ESI [M+H-tBu]=219.3.

(vii) Preparation of Compound 9

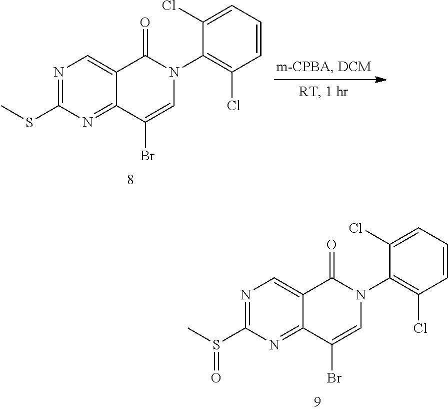 [SMILES: CSc1ncc2c(=O)n(-c3c(Cl)cccc3Cl)cc(Br)c2n1 ; CS(=O)c1ncc2c(=O)n(-c3c(Cl)cccc3Cl)cc(Br)c2n1]

To a solution of 8-bromo-6-(2,6-dichlorophenyl)-2-methylsulfanyl-pyrido[4,3-d]pyrimidin-5-one (50 mg, 119.87 μmol, 1.0 eq.) in DCM (2 mL) was added m-CPBA (36.50 mg, 179.81 μmol, 85% purity, 1.5 eq) at 0° C. The mixture was stirred at 20° C. for 1 hr and then used into next step without further purification. ESI [M+H]=432.0/434.0.

(viii) Preparation of Compound 10

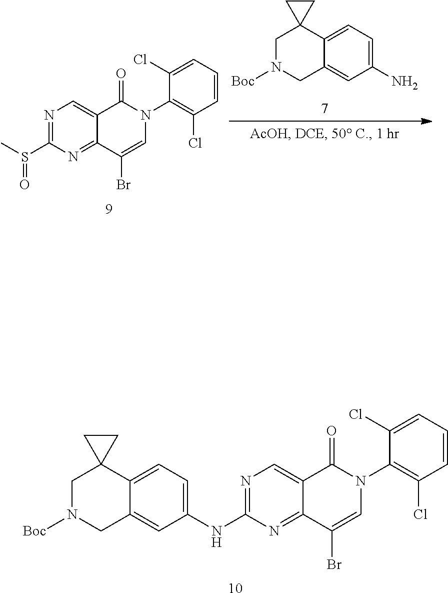

To a solution of 8-bromo-6-(2,6-dichlorophenyl)-2-methylsulfinyl-pyrido[4,3-d]pyrimidin-5-one (50 mg, 115.44 μmol, 1.0 eq.) in DCE (5 mL) was added tert-butyl 7-aminospiro[1,3-dihydroisoquinoline-4,1′-cyclopropane]-2-carboxylate (63.35 mg, 230.89 μmol, 2.0 eq.) and AcOH (41.60 mg, 692.67 μmol, 6.0 eq.). The mixture was stirred at 50° C. for 1 hr. The mixture was concentrated, diluted with Na₂CO₃ (3 mL), and then extracted with ethyl acetate (5 mL×3). The combined organic layers was dried over Na₂SO₄, filtered and concentrated to give a residue. The residue was purified by prep-TLC (SiO2, petroleum ether: ethyl acetate=5:1) to give tert-butyl 7-[[8-bromo-6-(2,6-dichlorophenyl)-5-oxo-pyrido[4,3-d]pyrimidin-2-yl]amino]spiro[1,3-dihydroisoquinoline-4,1′-cyclopropane]-2-carboxylate (50 mg, 77.72 μmol, 67.32% yield) as a yellow oil. ESI [M+H]=641.9/643.9.

### (ix) Preparation of Compound P19

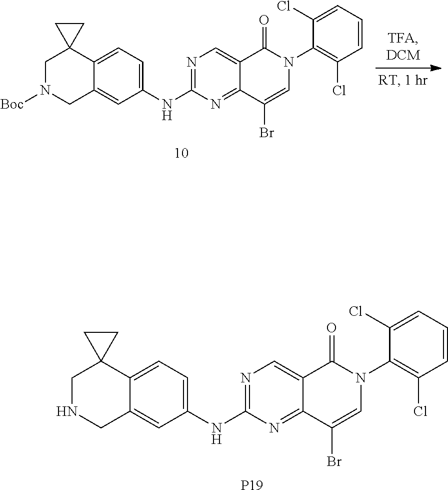

A mixture of tert-butyl 7-[[8-bromo-6-(2,6-dichlorophenyl)-5-oxo-pyrido[4,3-d]pyrimidin-2-yl]amino]spiro[1,3-dihydroisoquinoline-4,1′-cyclopropane]-2-carboxylate (20 mg, 31.09 μmol, 1.0 eq.) in TFA/DCM (2 mL, v/v=3/1) was stirred at 20° C. for 1 hr. The mixture was concentrated and purified by prep-HPLC (column: Phenomenex Luna C18 150*30 mm*5 μm; mobile phase: [water(0.1% TFA)-ACN];B %: 5%-45%, 8 min) to give 8-bromo-6-(2,6-dichlorophenyl)-2-(spiro[2,3-dihydro-1H-isoquinoline-4,1′-cyclopropane]-7-ylamino)pyrido[4,3-d]pyrimidin-5-one (8.59 mg, 13.07 μmol, 42.95% yield, 98.795% purity, TFA) as a light yellow solid. ¹H NMR (400 MHz, DMSO-d₆) δ 10.69 (br s, 1H), 9.15-9.29 (m, 3H), 8.46 (s, 1H), 8.14 (br s, 1H), 7.75 (d, J=8.13 Hz, 3H), 7.55-7.66 (m, 1H), 6.89 (d, J=8.63 Hz, 1H), 4.43 (br s, 2H), 3.28 (br s, 2H), 1.06-1.13 (m, 4H). ESI [M+H]=541.9/544.

### Example 22: Preparation of Compound P20

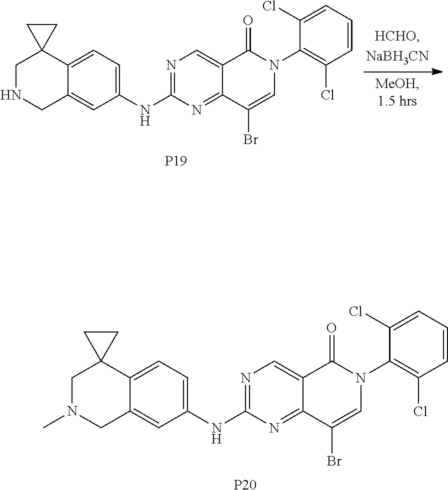

To a solution of 8-bromo-6-(2,6-dichlorophenyl)-2-(spiro[2,3-dihydro-1H-isoquinoline-4,1′-cyclopropane]-7-ylamino)pyrido[4,3-d]pyrimidin-5-one (20 mg, 30.43 μmol, 1.0 eq., TFA) in MeOH (2 mL) was added HCHO (7.41 mg, 91.29 μmol, 37% purity, 3.0 eq.) and DIEA (3.93 mg, 30.43 μmol, 1.0 eq.). The mixture was stirred at 20° C. for 0.5 hr followed by addition of NaBH₃CN (4 mg, 60.86 μmol, 2.0 eq). The mixture was stirred at 20° C. for 1 hr. The reaction mixture was quenched by addition sat.aq. Na₂CO₃ (3 mL), and extracted with DCM (2 mL×3). The combined organic layers were dried over Na₂SO₄, filtered and concentrated to give a residue. The residue was purified by prep-HPLC (column: Phenomenex Luna C18 150*30 mm*5 μm; mobile phase: [water(0.1% TFA)-ACN]; B %: 10%-50%, 8 min) to give 8-bromo-6-(2,6-dichlorophenyl)-2-[(2-methylspiro[1,3-dihydroisoquinoline-4,1′-cyclopropane]-7-yl)amino]pyrido[4,3-d]pyrimidin-5-one (4.42 mg, 6.58 μmol, 21.64% yield, 94.528% purity, TFA) as a yellow solid. ¹H NMR (400 MHz, DMSO-d₆) δ 10.72 (br s, 1H), 10.24 (br s, 1H), 9.19 (s, 1H), 8.46 (s, 1H), 8.22 (br s, 1H), 7.75 (d, J=8.13 Hz, 3H), 7.57-7.66 (m, 1H), 6.90 (d, J=8.75 Hz, 1H), 4.39-4.66 (m, 2H), 3.23 (br d, J=12.13 Hz, 2H), 2.96 (br d, J=2.63 Hz, 3H), 0.96-1.40 (m, 4H). ESI [M+H]=555.9/558.0.

### Example 23: Preparation of Compound P25

### (i) Preparation of Compound 2

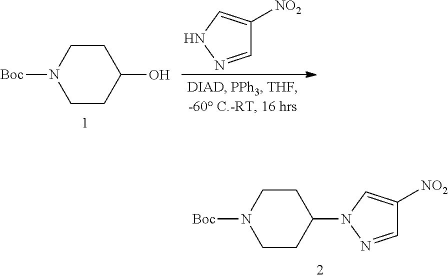 [SMILES: CC(C)(C)OC(=O)N1CCC(O)CC1 ; O=[N+]([O-])c1cn[nH]c1 ; CC(C)(C)OC(=O)N1CCC(n2cc([N+](=O)[O-])cn2)CC1]

To a solution of 4-nitro-1H-pyrazole (10 g, 88.44 mmol, 1.0 eq.), tert-butyl 4-hydroxypiperidine-1-carboxylate (17.80 g, 88.44 mmol, 1.0 eq.) and PPh₃ (34.79 g, 132.66 mmol, 1.5 eq.) in THE (400 mL) was added DIAD (26.82 g, 132.66 mmol, 1.5 eq.) dropwise at −60° C. under N₂. The mixture was allowed to warm to 20° C. and stirred for 16 hrs. Then the mixture was diluted with EtOAc (200 mL), washed with water (200 mL)/brine (200 mL), dried, filtered and concentrated. The residue was purified through silica gel column chromatography (petroleum ether: ethyl acetate=10:1 to 1:1) to give tert-butyl 4-(4-nitropyrazol-1-yl)piperidine-1-carboxylate (24 g, 80.99 mmol, 91.58% yield) as a white solid. LCMS: ESI [M+H-Boc]=197.3

### (ii) Preparation of Compound 3

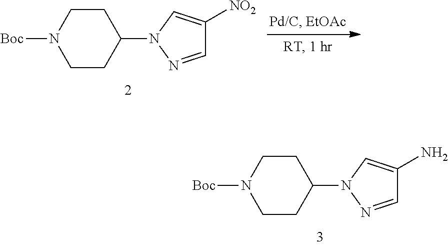 [SMILES: CC(C)(C)OC(=O)N1CCC(n2cc([N+](=O)[O-])cn2)CC1 ; CC(C)(C)OC(=O)N1CCC(n2cc(N)cn2)CC1]

A mixture of tert-butyl 4-(4-nitropyrazol-1-yl)piperidine-1-carboxylate (8 g, 27.00 mmol, 1 eq.), Pd/C (2 g, 10% purity) in EtOAc (80 mL) was degassed and purged with H₂ for 3 times, and then the mixture was stirred at 20° C. for 2 hrs under H₂ (15 Psi) atmosphere. The reaction mixture was filtered and concentrated. The residue was purified by column chromatography (SiO₂, petroleum ether/ethyl acetate=10/1 to 0/1) to give tert-butyl 4-(4-aminopyrazol-1-yl)piperidine-1-carboxylate (4 g, 14.40 mmol, 53.34% yield, 95.88% purity) as a red oil. LCMS: ESI [M+H-tBu]=211.3.

(iii) Preparation of Compound 5

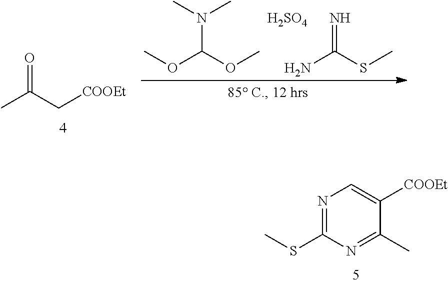

A mixture of ethyl 3-oxobutanoate (61.80 g, 474.87 mmol, 1.0 eq.) and 1,1-dimethoxy-N,N-dimethyl-methanamine (68.17 g, 572.10 mmol, 1.20 eq.) was heated to 80° C. for 10 min. The mixture was cooled to 20° C. and followed by the addition of 2-methylisothiourea; sulfuric acid (66.10 g, 237.43 mmol, 0.5 eq.). The mixture was heated at 85° C. for 12 hrs and then concentrated and the residue was dissolved in EtOAc (800 mL), washed with water (800 mL), sat.aq.NaHCO₃ (400 mL), brine (400 mL), dried and concentrated to give ethyl 4-methyl-2-methylsulfanyl-pyrimidine-5-carboxylate (76.8 g, crude) as a yellow oil. ESI [M+H]=213.2.

### (iv) Preparation of Compound 6

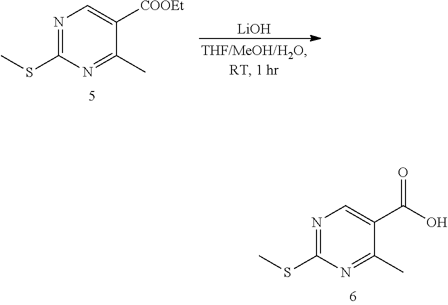

A solution of LiOH·H₂O (27.72 g, 660.63 mmol, 1.85 eq.) in H₂O (278 mL) was added into a solution of ethyl 4-methyl-2-methylsulfanyl-pyrimidine-5-carboxylate (75.8 g, 357.10 mmol, 1.0 eq.) in THE (278 mL)/MeOH (92 mL). The mixture was stirred at 20° C. for 1 hr and then concentrated to remove the solvent. The residual aqueous layer was extracted with methyl tert-butyl ether (500 mL) and then acidified with 1N HCl to adjust pH=2. The precipitate was filtered and the filter cake was dried to give 4-methyl-2-methylsulfanyl-pyrimidine-5-carboxylic acid (38 g, 201.66 mmol, 56.47% yield, 97.76% purity) as a yellow solid. ESI [M+H]=185.1.

### (v) Preparation of Compound 7

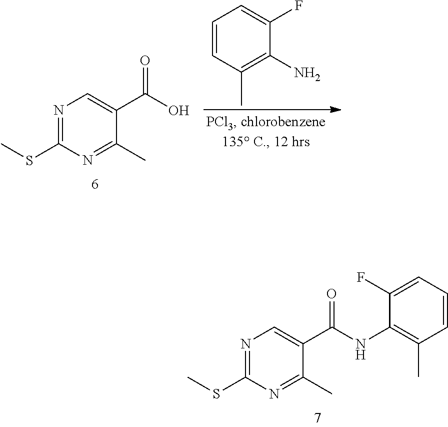

A mixture of 4-methyl-2-methylsulfanyl-pyrimidine-5-carboxylic acid (3 g, 16.29 mmol, 1.0 eq.) and 2-fluoro-6-methyl-aniline (2.04 g, 16.29 mmol, 1.0 eq.) in Chlorobenzene (45 mL) was degassed and purged with N₂ for 3 times and followed by the addition of PCl₃ (2.24 g, 16.29 mmol, 1.0 eq.) at 20° C. Then the mixture was stirred at 135° C. for 12 hrs under N₂ atmosphere. The mixture was concentrated and the residue was poured into sat.aq.Na₂CO₃ (100 mL) and extracted with 2-methyl-THF (50 mL×3). The organic phase was dried and concentrated. The residue was triturated with EtOAc (6 mL) to give N-(2-fluoro-6-methyl-phenyl)-4-methyl-2-methylsulfanyl-pyrimidine-5-carboxamic (2.3 g, 6.23 mmol, 38.23% yield, 78.87% purity) as a yellow solid. ESI [M+H]=292.2.

### (vi) Preparation of Compound 8

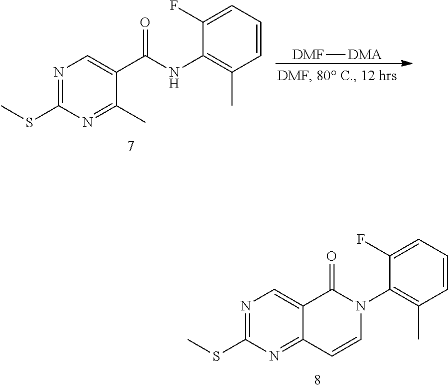

To a solution of N-(2-fluoro-6-methyl-phenyl)-4-methyl-2-methylsulfanyl-pyrimidine-5-carboxamide (2.3 g, 7.89 mmol, 1.0 eq.) in DMF (57 mL) was added DMF-DMA (5.64 g, 47.37 mmol, 6.29 mL, 6 eq.). The mixture was stirred at 80° C. for 12 hrs. The mixture was concentrated and purified by triturated with EtOH (8 mL) to give 6-(2-fluoro-6-methyl-phenyl)-2-methylsulfanyl-pyrido[4,3-d]pyrimidin-5-one (2.13 g, 6.35 mmol, 80.42% yield, 89.82% purity) as a white solid. ESI [M+H]=302.3.

(vii) Preparation of Compound 9

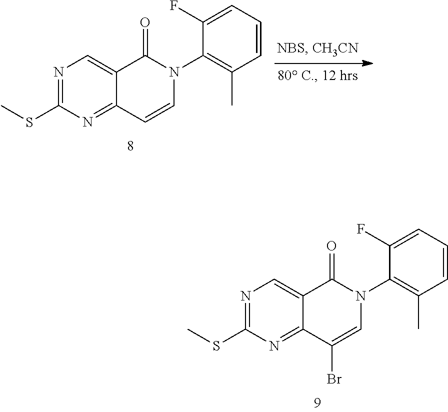 [SMILES: CSc1ncc2c(=O)n(-c3c(C)cccc3F)ccc2n1 ; CSc1ncc2c(=O)n(-c3c(C)cccc3F)cc(Br)c2n1]

To a solution of 6-(2-fluoro-6-methyl-phenyl)-2-methylsulfanyl-pyrido[4,3-d]pyrimidin-5-one (2.13 g, 7.07 mmol, 1.0 eq.) in CH₃CN (60 mL) was added NBS (1.89 g, 10.60 mmol, 1.5 eq.). The mixture was stirred at 80° C. for 12 hrs. The mixture was concentrated and the crude product was triturated with EtOH (10 mL) to give 8-bromo-6-(2-fluoro-6-methyl-phenyl)-2-methylsulfanyl-pyrido[4,3-d]pyrimidin-5-one (2.3 g, 5.51 mmol, 78.02% yield, 91.17% purity) as a yellow solid. ESI [M+H]=380.2/382.1.

(viii) Preparation of Compound 10

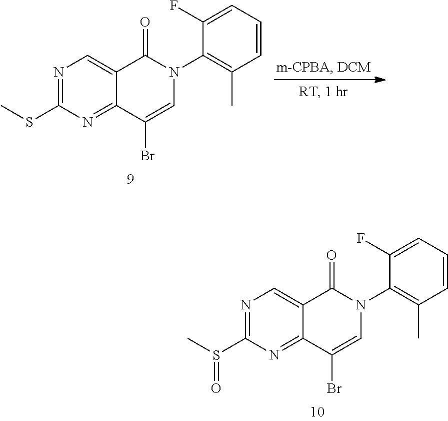 [SMILES: CSc1ncc2c(=O)n(-c3c(C)cccc3F)cc(Br)c2n1 ; Cc1cccc(F)c1-n1cc(Br)c2nc(S(C)=O)ncc2c1=O]

To a solution of 8-bromo-6-(2-fluoro-6-methyl-phenyl)-2-methylsulfanyl-pyrido[4,3-d]pyrimidin-5-one (1 g, 2.63 mmol, 1.0 eq.) in DCM (43 mL) was added m-CPBA (694.11 mg, 3.42 mmol, 85% purity, 1.3 eq.) at 0° C. Then the mixture was stirred at 20° C. for 1 hr. The mixture was used into next step without further purification. ESI [M+H]=398.1/396.1.

### (ix) Preparation of Compound 11

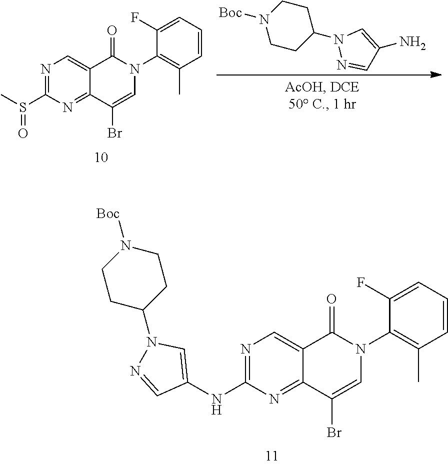 [SMILES: Cc1cccc(F)c1-n1cc(Br)c2nc(S(C)=O)ncc2c1=O ; CC(C)(C)OC(=O)N1CCC(n2cc(N)cn2)CC1 ; Cc1cccc(F)c1-n1cc(Br)c2nc(Nc3cnn(C4CCN(C(=O)OC(C)(C)C)CC4)c3)ncc2c1=O]

To a solution of 8-bromo-6-(2-fluoro-6-methyl-phenyl)-2-methylsulfinyl-pyrido[4,3-d]pyrimidin-5-one (1 g, 2.52 mmol, 1.0 eq.) in DCE (60 mL) was added tert-butyl 4-(4-aminopyrazol-1-yl)piperidine-1-carboxylate (1.01 g, 3.79 mmol, 1.5 eq.). Then AcOH (909.34 mg, 15.14 mmol, 6 eq.) was added. The mixture was stirred at 50° C. for 1 hr. The mixture was concentrated and the residue was purified by prep-HPLC (column: Kromasil C18 (250*50 mm*10 um); mobile phase: [water (10 mM NH₄HCO₃)-ACN]; B %: 45%-65%, 10 min) to give tert-butyl 4-[4-[[8-bromo-6-(2-fluoro-6-methyl-phenyl)-5-oxo-pyrido[4,3-d]pyrimidin-2-yl]amino]pyrazol-1-yl]piperidine-1-carboxylate (0.7 g, 1.17 mmol, 46.35% yield) as a yellow solid. ESI [M+H]=542.2/544.2.

### (x) Preparation of Compound P25

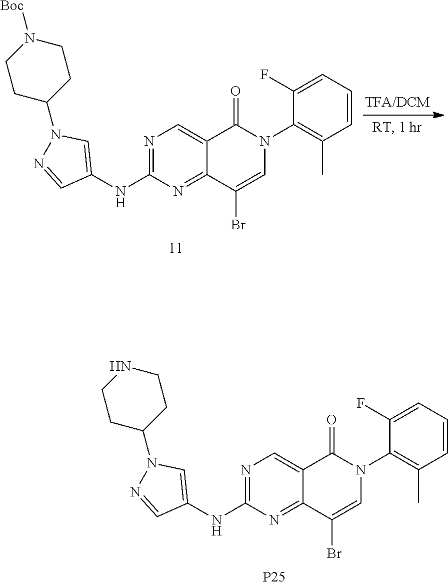

To a solution of tert-butyl 4-[4-[[8-bromo-6-(2-fluoro-6-methyl-phenyl)-5-oxo-pyrido[4,3-d]pyrimidin-2-yl]amino]pyrazol-1-yl]piperidine-1-carboxylate (150 mg, 250.64 μmol, 1.0 eq.) in DCM (6 mL) was added TFA (2 mL). The mixture was stirred at 20° C. for 1 hr. The mixture was concentrated and purified by prep-HPLC (column: Phenomenex Luna C18 150*30 mm*5 μm; mobile phase: [water(0.1% TFA)-ACN]; B %: 10%-40%, 9 min) to give 8-bromo-6-(2-fluoro-6-methyl-phenyl)-2-[[1-(4-piperidyl)pyrazol-4-yl]amino]pyrido[4,3-d]pyrimidin-5-one (34.37 mg, 68.97 μmol, 84.47% yield, 100% purity, TFA) as a yellow solid. ¹H NMR (400 MHz, DMSO-d₆) δ 10.69 (s, 1H), 9.10 (s, 1H), 8.72 (br s, 1H), 8.27-8.49 (m, 2H), 7.66-8.04 (m, 1H), 7.47 (dt, J=5.82, 7.97 Hz, 1H), 7.21-7.35 (m, 2H), 4.46-4.62 (m, 1H), 3.44 (br d, J=12.76 Hz, 2H), 3.11 (q, J=11.30 Hz, 2H), 1.98-2.30 (m, 7H). ESI [M+H]=498.1/500.1.

### Example 24: Preparation of Compound P26

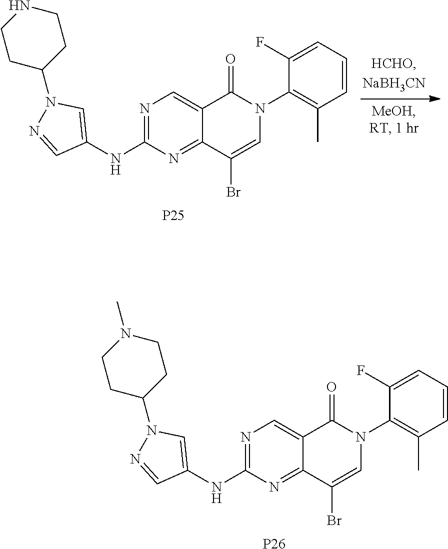

To a solution of 8-bromo-6-(2-fluoro-6-methyl-phenyl)-2-[[1-(4-piperidyl)pyrazol-4-yl]amino]pyrido[4,3-d]pyrimidin-5-one (100 mg, 163.30 μmol, 1 eq., TFA) in MeOH (4 mL) was added DIEA (21.11 mg, 163.30 μmol, 1.0 eq.) and HCHO (39.76 mg, 489.90 μmol, 37% purity, 3.0 eq.). The mixture was stirred at 20° C. for 0.5 hr. Then NaBH₃CN (10.26 mg, 163.30 μmol, 1.0 eq.) was added. The mixture was stirred at 20° C. for 1 hr. The mixture was concentrated and the residue was purified by prep-HPLC (column: Phenomenex Luna C18 150*30 mm*5 μm; mobile phase: [water (0.1% TFA)-ACN]; B %: 12%-42%, 9 min) to give 8-bromo-6-(2-fluoro-6-methyl-phenyl)-2-[[1-(1-methyl-4-piperidyl)pyrazol-4-yl]amino]pyrido[4,3-d]pyrimidin-5-one (56.6 mg, 90.36 μmol, 55.33% yield, 100% purity, TFA) as a yellow solid. ¹H NMR (400 MHz, DMSO-d₆) δ 10.46-10.80 (m, 1H), 9.57-9.95 (m, 1H), 9.10 (s, 1H), 8.27-8.50 (m, 2H), 7.66-8.09 (m, 1H), 7.40-7.55 (m, 1H), 7.21-7.35 (m, 2H), 4.41-4.63 (m, 1H), 3.59 (br d, J=11.63 Hz, 2H), 3.10-3.25 (m, 2H), 2.83 (br s, 3H), 2.06-2.36 (m, 7H). ESI [M+H]=512.1/514.1.

### Example 25: Preparation of Compound P14

### (i) Preparation of Compound 2

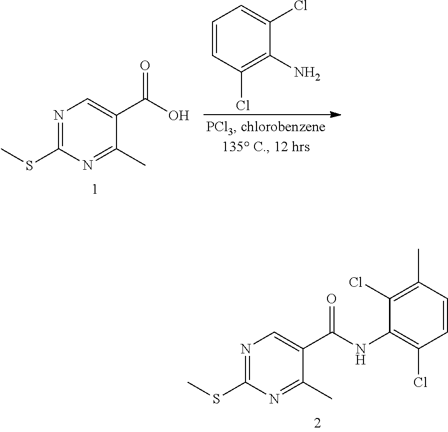 [SMILES: CSc1ncc(C(=O)O)c(C)n1 ; Nc1c(Cl)cccc1Cl ; CSc1ncc(C(=O)Nc2c(Cl)ccc(C)c2Cl)c(C)n1]

To a solution of 4-methyl-2-methylsulfanyl-pyrimidine-5-carboxylic acid (3.14 g, 17.04 mmol, 1.0 eq.) and 2,6-dichloro-3-methylaniline (3 g, 17.04 mmol, 1.0 eq.) in chlorobenzene (30 mL) was added PCl₃ (2.34 g, 17.04 mmol, 1.0 eq.). The mixture was stirred at 135° C. for 12 hrs. The mixture was concentrated and the residue was poured into sat. aq. Na₂CO₃ (100 mL) and extracted with 2-methyl-THF (50 mL×3). The organic phase was dried and concentrated. The residue was triturated with EtOH (6 mL) to give N-(2,6-dichloro-3-methyl-phenyl)-4-methyl-2-methylsulfanyl-pyrimidine-5-carboxamide (2.3 g, 6.72 mmol, 39.44% yield) as a yellow solid. ESI [M+H]=342.1/344.1.

### (ii) Preparation of Compound 3

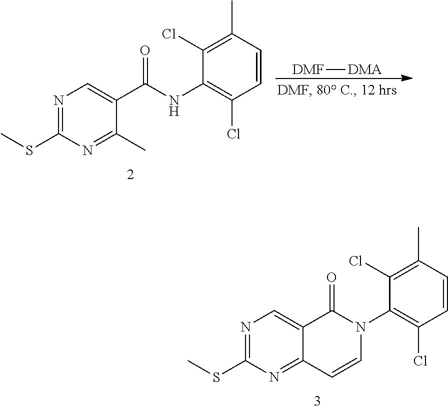

To a solution of N-(2,6-dichloro-3-methyl-phenyl)-4-methyl-2-methylsulfanyl-Pyrimidine-5-carboxamide (2.1 g, 6.14 mmol, 1.0 eq.) in DMF (50 mL) was added DMF-DMA (4.39 g, 36.82 mmol, 6.0 eq.). The mixture was stirred at 80° C. for 12 hrs. The mixture was concentrated and triturated with EtOH (6 mL) to give 6-(2,6-dichloro-3-methyl-phenyl)-2-methylsulfanyl-pyrido[4,3-d]pyrimidin-5-one (1.3 g, 3.69 mmol, 60.15% yield) as a yellow solid. ESI [M+H]=352.0/354.0.

(iii) Preparation of Compound 4

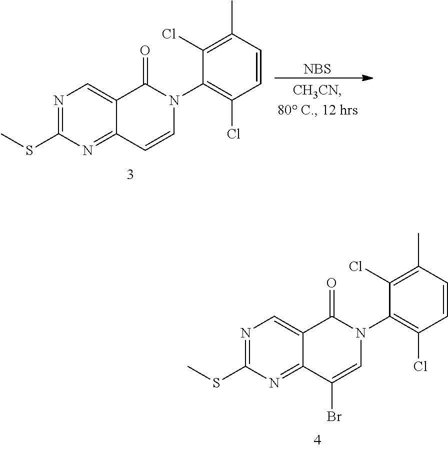

A mixture of 6-(2,6-dichloro-3-methyl-phenyl)-2-methylsulfanyl-pyrido[4,3-d]pyrimidin-5-one (1.2 g, 3.41 mmol, 1.0 eq.) and NBS (909.51 mg, 5.11 mmol, 1.5 eq.) in CH₃CN (12 mL) was degassed and purged with N₂ for 3 times, and then the mixture was stirred at 80° C. for 12 hrs under N₂ atmosphere. The mixture was concentrated and then triturated with EtOH (6 mL) to give 8-bromo-6-(2,6-dichloro-3-methyl-phenyl)-2-methylsulfanyl-pyrido[4,3-d]pyrimidin-5-one (1.2 g, 2.78 mmol, 81.70% yield) as a light yellow solid. ESI [M+H]=431.8/429.8.

### (iv) Preparation of Compound 5

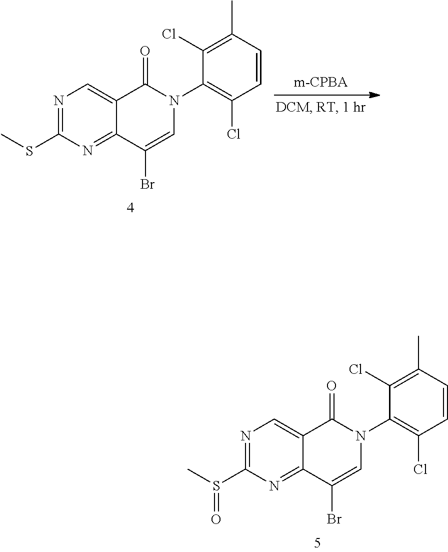 [SMILES: CSc1ncc2c(=O)n(-c3c(Cl)ccc(C)c3Cl)cc(Br)c2n1 ; Cc1ccc(Cl)c(-n2cc(Br)c3nc(S(C)=O)ncc3c2=O)c1Cl]

To a solution of 8-bromo-6-(2,6-dichloro-3-methyl-phenyl)-2-methylsulfanyl-pyrido[4,3-d]pyrimidin-5-one (100 mg, 231.95 μmol, 1.0 eq.) in DCM (2 mL) was added m-CPBA (70.63 mg, 347.92 μmol, 85% purity, 1.5 eq.) at 0° C. The mixture was stirred at 20° C. for 1 hr. The mixture was concentrated to give 8-bromo-6-(2,6-dichloro-3-methyl-phenyl)-2-methylsulfinyl-pyrido[4,3-d]pyrimidin-5-one (100 mg, crude) as a yellow solid and used into next step without further purification. ESI [M+H]=447.9/445.9.

### (v) Preparation of Compound 6

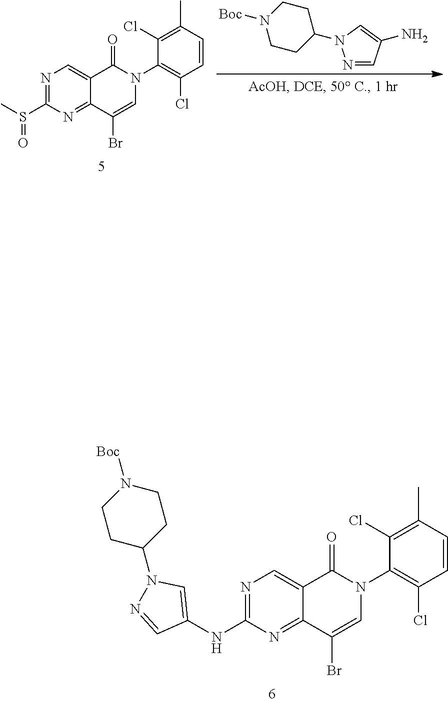 [SMILES: Cc1ccc(Cl)c(-n2cc(Br)c3nc(S(C)=O)ncc3c2=O)c1Cl ; CC(C)(C)OC(=O)N1CCC(n2cc(N)cn2)CC1 ; Cc1ccc(Cl)c(-n2cc(Br)c3nc(Nc4cnn(C5CCN(C(=O)OC(C)(C)C)CC5)c4)ncc3c2=O)c1Cl]

To a solution of 8-bromo-6-(2,6-dichloro-3-methyl-phenyl)-2-methylsulfinyl-pyrido[4,3-d]pyrimidin-5-one (100 mg, 223.65 μmol, 1.0 eq.) in DCE (5 mL) was added tert-butyl 4-(4-aminopyrazol-1-yl)piperidine-1-carboxylate (119.13 mg, 447.29 μmol, 2.0 eq.) and AcOH (40.29 mg, 670.94 μmol, 38.37 μL, 3.0 eq.). The mixture was stirred at 50° C. for 1 hr. The mixture was concentrated and purified by prep-TLC (SiO2, petroleum ether: ethyl acetate=1:1) to give tert-butyl 4-[4-[[8-bromo-6-(2,6-dichloro-3-methyl-phenyl)-5-oxo-pyrido[4,3-d]pyrimidin-2-yl]amino]pyrazol-1-yl]piperidine-1-carboxylate (130 mg, 200.20 μmol, 89.51% yield) as a yellow oil. ESI [M+H]=650.1/648.1.

### (vi) Preparation of Compound P14

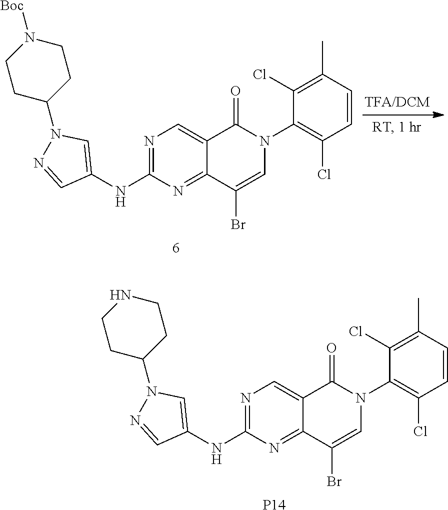

To a solution of tert-butyl 4-[4-[[8-bromo-6-(2,6-dichloro-3-methyl-phenyl)-5-oxo-pyrido[4,3-d]pyrimidin-2-yl]amino]pyrazol-1-yl]piperidine-1-carboxylate (130 mg, 200.20 μmol, 1.0 eq.) in DCM (3 mL) was added TFA (1 mL). The mixture was stirred at 20° C. for 1 hr. The mixture was concentrated and purified by prep-HPLC (column: Phenomenex Luna C18 150*30 mm*5 μm; mobile phase: [water (TFA)-ACN]; B %: 5%-45%, 8 min) to give 8-bromo-6-(2,6-dichloro-3-methyl-phenyl)-2-[[1-(4-piperidyl)pyrazol-4-yl]amino]pyrido[4,3-d]pyrimidin-5-one (21.65 mg, 32.64 μmol, 43.30% yield, purity, TFA) as a yellow solid. ¹H NMR (400 MHz, DMSO-d₆) δ 10.74 (s, 1H), 9.10 (s, 1H), 8.74 (br s, 1H), 8.46 (s, 1H), 8.40 (s, 1H), 7.81 (s, 1H), 7.57-7.68 (m, 2H), 4.47-4.60 (m, 1H), 3.42 (br s, 2H), 3.11 (br d, J=10.01 Hz, 2H), 2.42 (s, 3H), 2.24 (br d, J=10.76 Hz, 2H), 2.03-2.15 (m, 2H). ESI [M+H]=549.9/548.0.

### Example 26: Preparation of Compound P15

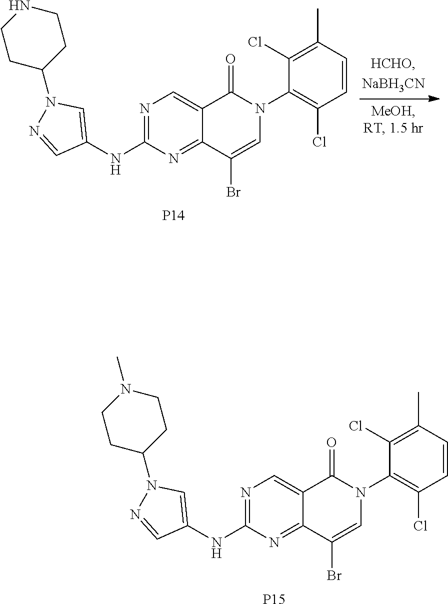

To a solution of 8-bromo-6-(2,6-dichloro-3-methyl-phenyl)-2-[[1-(4-piperidyl)pyrazol-4-yl]amino]pyrido[4,3-d]pyrimidin-5-one (50 mg, 75.38 μmol, 1.0 eq., TFA) in MeOH (2 mL) was added HCHO (18.35 mg, 226.15 μmol, 37% purity, 3.0 eq.) and DIEA (9.74 mg, 75.38 μmol, 13.13 μL, 1.0 eq.). The mixture was stirred at 20° C. for 0.5 hr, followed by the addition of NaBH₃CN (4.74 mg, 75.38 μmol, 1.0 eq.). The mixture was stirred at 20° C. for 1 hr. The reaction mixture was quenched by sat.aq. Na₂CO₃ (3 mL), and extracted with DCM (1 mL×3). The combined organic layers were dried over Na₂SO₄, filtered and concentrated. The residue was purified by prep-HPLC (column: Phenomenex luna C18 100*40 mm*5 μm; mobile phase: [water(TFA)-ACN]; B %: 15%-60%, 8 min) to give 8-bromo-6-(2,6-dichloro-3-methyl-phenyl)-2-[[1-(1-methyl-4-piperidyl)pyrazol-4-yl]amino]pyrido[4,3-d]pyrimidin-5-one (9.28 mg, 13.70 μmol, 18.18% yield, 100% purity, TFA) as a yellow solid. ¹H NMR (400 MHz, DMSO-d₆) δ 10.52-10.82 (m, 1H), 9.61 (br s, 1H), 9.07-9.21 (m, 1H), 8.31-8.48 (m, 2H), 7.82 (s, 1H), 7.54-7.71 (m, 2H), 4.49 (br s, 1H), 3.59 (br d, J=11.38 Hz, 2H), 3.17 (br s, 2H), 2.83 (s, 3H), 2.42 (s, 3H), 2.31 (br d, J=13.01 Hz, 2H), 2.05-2.20 (m, 2H). ESI [M+H]=563.9/562.0.

### Example 27: Preparation of Compound P16

### (i) Preparation of Compound 2

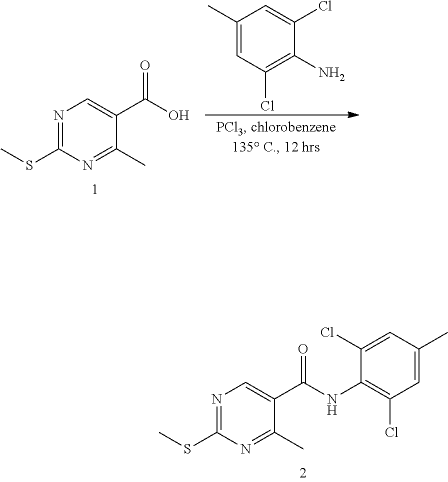

To a solution of 4-methyl-2-methylsulfanyl-pyrimidine-5-carboxylic acid (3.14 g, 17.04 mmol, 1.0 eq.) and 2,6-dichloro-4-methylaniline (3 g, 17.04 mmol, 1.0 eq.) in Chlorobenzene (30 mL) was added PCl₃ (2.34 g, 17.04 mmol, 1.0 eq.). The mixture was stirred at 135° C. for 12 hrs. The mixture was concentrated and the residue was poured into sat.aq.Na₂CO₃ (100 mL) and extracted with 2-methyl-THF (50 mL×3). The organic phase was dried and concentrated. The residue was triturated with EtOH (6 mL) to give N-(2,6-dichloro-4-methyl-phenyl)-4-methyl-2-methylsulfanyl-pyrimidine-5-carboxamide (2.5 g, 7.30 mmol, 42.86% yield) as a yellow solid. ESI [M+H]=342.0/344.0.

### (ii) Preparation of Compound 3

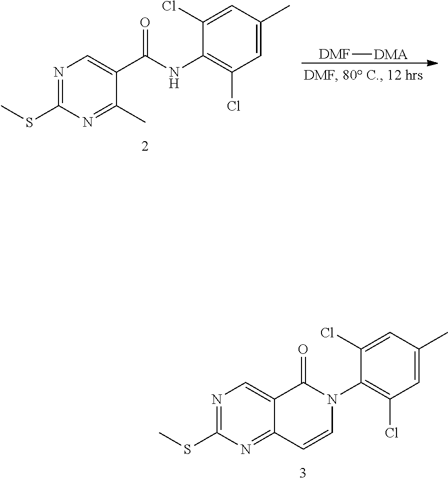

To a solution of N-(2,6-dichloro-4-methyl-phenyl)-4-methyl-2-methylsulfanyl-pyrimidine-5-carboxamide (2.24 g, 6.55 mmol, 1.0 eq.) in DMF (50 mL) was added DMF-DMA (4.68 g, 39.27 mmol, 5.22 mL, 6.0 eq.). The mixture was stirred at 80° C. for 12 hrs. The mixture was concentrated and triturated with EtOH (6 mL) to give 6-(2,6-dichloro-4-methyl-phenyl)-2-methylsulfanyl-pyrido[4,3-d]pyrimidin-5-one (1.8 g, 5.11 mmol, 78.08% yield) as a light yellow solid. ESI [M+H]=352.0/354.0.

(iii) Preparation of Compound 4

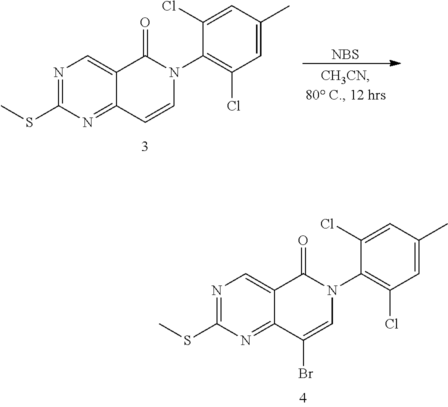

A mixture of 6-(2,6-dichloro-4-methyl-phenyl)-2-methylsulfanyl-pyrido[4,3-d]pyrimidin-5-one (1.7 g, 4.83 mmol, 1.0 eq.) and NBS (1.29 g, 7.24 mmol, 1.5 eq.) in CH₃CN (20 mL) was degassed and purged with N₂ for 3 times, and then the mixture was stirred at 80° C. for 12 hrs under N₂ atmosphere. The mixture was concentrated and triturated with EtOH (6 mL) to give 8-bromo-6-(2,6-dichloro-4-methyl-phenyl)-2-methylsulfanyl-pyrido[4,3-d]pyrimidin-5-one (2 g, 4.64 mmol, 96.12% yield) as a light yellow solid. ESI [M+H]=431.9/429.9.

### (iv) Preparation of Compound 5

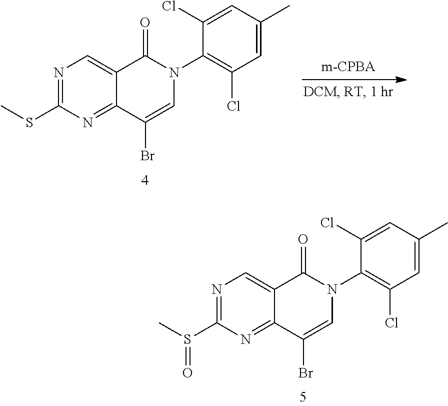

To a solution of 8-bromo-6-(2,6-dichloro-4-methyl-phenyl)-2-methylsulfanyl-pyrido[4,3-d]pyrimidin-5-one (100 mg, 231.95 μmol, 1.0 eq.) in DCM (2 mL) was added m-CPBA (70.63 mg, 347.92 μmol, 85% purity, 1.5 eq.) at 0° C. The mixture was stirred at 20° C. for 1 hr. The mixture was concentrated to give 8-bromo-6-(2,6-dichloro-4-methyl-phenyl)-2-methylsulfinyl-pyrido[4,3-d]pyrimidin-5-one (100 mg, crude) as a yellow oil and used into next step without further purification. ESI [M+H]=447.9/445.9.

### (v) Preparation of Compound 6

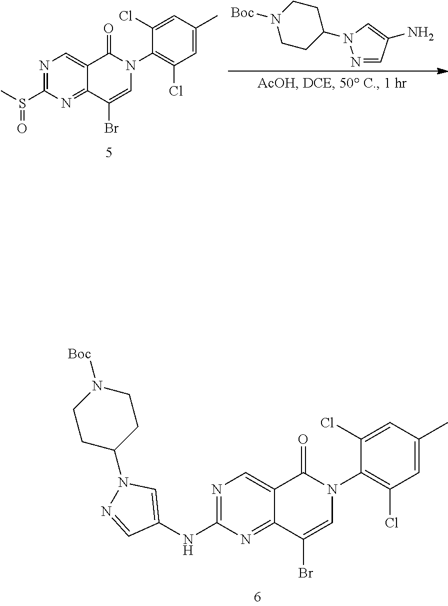

To a solution of 8-bromo-6-(2,6-dichloro-4-methyl-phenyl)-2-methylsulfinyl-pyrido[4,3-d]pyrimidin-5-one (100 mg, 223.65 μmol, 1.0 eq.) in DCE (5 mL) was added tert-butyl 4-(4-aminopyrazol-1-yl)piperidine-1-carboxylate (119.13 mg, 447.29 μmol, 2.0 eq.) and AcOH (80.58 mg, 1.34 mmol, 76.75 μL, 6.0 eq.). The mixture was stirred at 50° C. for 1 hr. The mixture was concentrated and purified by prep-TLC (SiO₂, petroleum ether: ethyl acetate=1:1) to give tert-butyl 4-[4-[[8-bromo-6-(2,6-dichloro-4-methyl-phenyl)-5-oxo-pyrido[4,3-d]pyrimidin-2-yl]amino]pyrazol-1-yl]piperidine-1-carboxylate (120 mg, crude) as a yellow oil. ESI [M+H]=649.9/647.9.

### (vi) Preparation of Compound P16

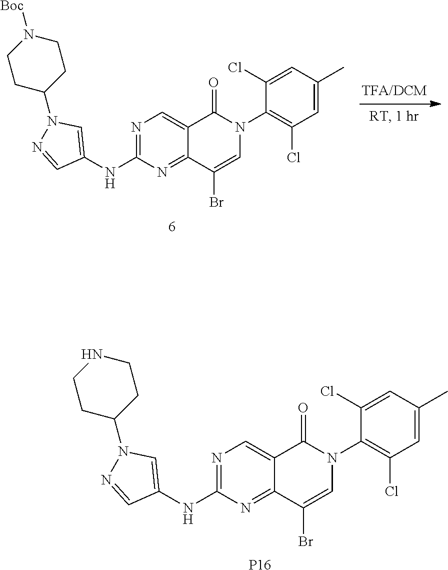

To a solution of tert-butyl 4-[4-[[8-bromo-6-(2,6-dichloro-4-methyl-phenyl)-5-oxo-pyrido[4,3-d]pyrimidin-2-yl]amino]pyrazol-1-yl]piperidine-1-carboxylate (120 mg, 184.80 μmol, 1 eq.) in DCM (4 mL) was added TFA (1 mL). The mixture was stirred at 20° C. for 1 hr. The mixture was concentrated and purified by prep-HPLC (column: Phenomenex Luna 80*30 mm*3 um; mobile phase: [water(TFA)-ACN]; B %: 15%-45%, 8 min) to give 8-bromo-6-(2,6-dichloro-4-methyl-phenyl)-2-[[1-(4-piperidyl)pyrazol-4-yl]amino]pyrido[4,3-d]pyrimidin-5-one (27.90 mg, 42.06 μmol, 55.80% yield, 100% purity, TFA) as a yellow solid. ¹H NMR (400 MHz, DMSO-d₆) δ 10.73 (s, 1H), 9.10 (s, 1H), 8.78 (br s, 1H), 8.45 (s, 2H), 8.40 (s, 1H), 7.81 (s, 1H), 7.58 (s, 2H), 4.46-4.64 (m, 1H), 3.43 (br d, J=12.76 Hz, 2H), 3.11 (br d, J=7.63 Hz, 2H), 2.41 (s, 3H), 2.24 (br d, J=11.01 Hz, 2H), 2.02-2.17 (m, 2H). ESI [M+H]=549.9/548.0.

### Example 28: Preparation of Compound P17

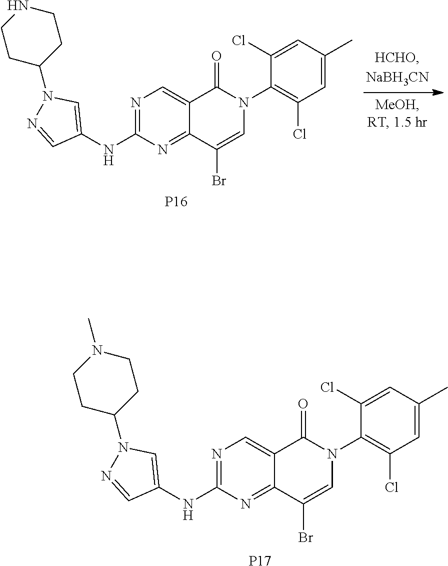

To a solution of 8-bromo-6-(2,6-dichloro-4-methyl-phenyl)-2-[[1-(4-piperidyl)pyrazol-4-yl]amino]pyrido[4,3-d]pyrimidin-5-one (60 mg, 90.46 μmol, 1.0 eq., TFA) in MeOH (2 mL) was added HCHO (22.03 mg, 271.38 μmol, 37% purity, 3.0 eq.) and DIEA (11.69 mg, 90.46 μmol, 15.76 μL, 1 eq.). The mixture was stirred at 20° C. for 0.5 hr followed by the addition of NaBH₃CN (5.68 mg, 90.46 μmol, 1 eq.). The mixture was stirred at 20° C. for 1 hr. The reaction mixture was quenched by sat.aq. Na₂CO₃ (3 mL), and extracted with DCM (1 mL×3). The combined organic layers were dried over Na₂SO₄, filtered and concentrated. The residue was purified by prep-HPLC (column: Phenomenex Luna 80*30 mm*3 um; mobile phase: [water (TFA)-ACN]; B %: 15%-45%, 8 min) to give 8-bromo-6-(2,6-dichloro-4-methyl-phenyl)-2-[[1-(1-methyl-4-piperidyl)pyrazol-4-yl]amino]pyrido[4,3-d]pyrimidin-5-one (47.24 mg, 69.75 μmol, 77.10% yield, 100% purity, TFA) as a yellow solid. ¹H NMR (400 MHz, DMSO-d₆) δ 10.51-10.83 (m, 1H), 9.74-10.03 (m, 1H), 9.10 (s, 1H), 8.29-8.50 (m, 2H), 7.83 (s, 1H), 7.58 (s, 2H), 4.43-4.62 (m, 1H), 3.59 (br d, J=11.51 Hz, 2H), 3.18 (br s, 2H), 2.83 (s, 3H), 2.41 (s, 3H), 2.31 (br d, J=12.88 Hz, 2H), 2.06-2.24 (m, 2H). ESI [M+H]=563.9/561.9.

### Example 29: Preparation of Compound P13

### (i) Preparation of Compound 2

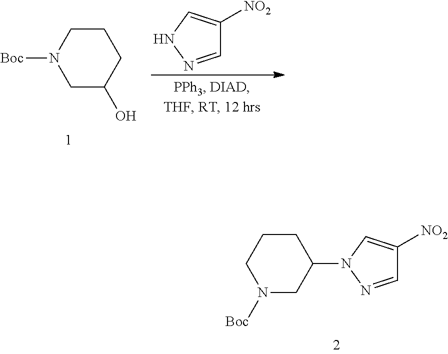 [SMILES: CC(C)(C)OC(=O)N1CCCC(O)C1 ; O=[N+]([O-])c1cn[nH]c1 ; CC(C)(C)OC(=O)N1CCCC(n2cc([N+](=O)[O-])cn2)C1]

A mixture of 4-nitro-1H-pyrazole (500 mg, 4.42 mmol, 1.0 eq.), tert-butyl 3-hydroxypiperidine-1-carboxylate (889.95 mg, 4.42 mmol, 1.0 eq.) and PPh₃ (1.74 g, 6.63 mmol, 1.5 eq.) in THF (30 mL) was degassed and purged with N₂ for 3 times, then DIAD (1.34 g, 6.63 mmol, 1.5 eq.) was added dropwise at −60° C. The mixture was stirred at 20° C. for 12 hrs. The mixture was concentrated and the residue was purified by prep-HPLC (neutral condition, column: Waters Xbridge Prep OBD C18 150*40 mm*10 μm; mobile phase: [water (NH₄HCO₃)-ACN]; B %: 30%-60%, 8 min) to give tert-butyl 3-(4-nitropyrazol-1-yl)piperidine-1-carboxylate (120 mg, 404.97 μmol, 9.16% yield) was obtained as a colorless oil. ESI [M+H-t]=241.1.

### (ii) Preparation of Compound 3

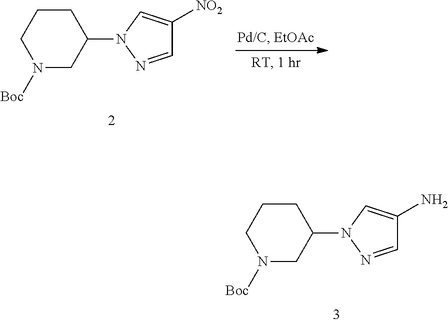 [SMILES: CC(C)(C)OC(=O)N1CCCC(n2cc([N+](=O)[O-])cn2)C1 ; CC(C)(C)OC(=O)N1CCCC(n2cc(N)cn2)C1]

A mixture of tert-butyl 3-(4-nitropyrazol-1-yl)piperidine-1-carboxylate (110 mg, 371.22 μmol, 1 eq.), Pd/C (110 mg, 10% purity) in EtOAc (10 mL) was degassed and purged with H₂ (15 Psi) for 3 times, and then the mixture was stirred at 30° C. for 1 hr under H₂ (15 Psi) atmosphere. The reaction mixture was filtered, and the filtrate was concentrated to give tert-butyl 3-(4-aminopyrazol-1-yl)piperidine-1-carboxylate (80 mg, crude) as a red oil which was used into next step without further purification. ESI [M+H]=267.2.

(iii) Preparation of Compound 5

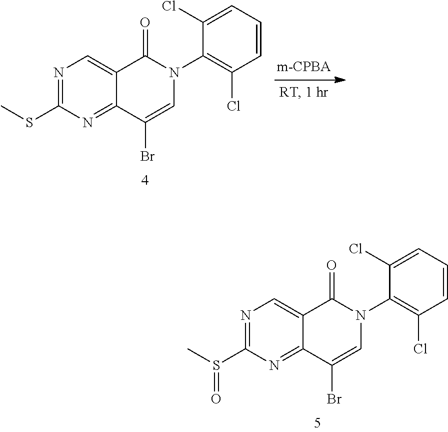 [SMILES: CSc1ncc2c(=O)n(-c3c(Cl)cccc3Cl)cc(Br)c2n1 ; CS(=O)c1ncc2c(=O)n(-c3c(Cl)cccc3Cl)cc(Br)c2n1]

To a solution of 8-bromo-6-(2,6-dichlorophenyl)-2-methylsulfanyl-pyrido[4,3-d]pyrimidin-5-one (60 mg, 143.85 μmol, 1.0 eq.) in DCM (2 mL) was added m-CPBA (43.81 mg, 215.77 μmol, 85% purity, 1.5 eq.) at 0° C. The mixture was stirred at 20° C. for 1 hr and used into next step without further purification. ESI [M+H]=433.9/431.9.

### (iv) Preparation of Compound 6

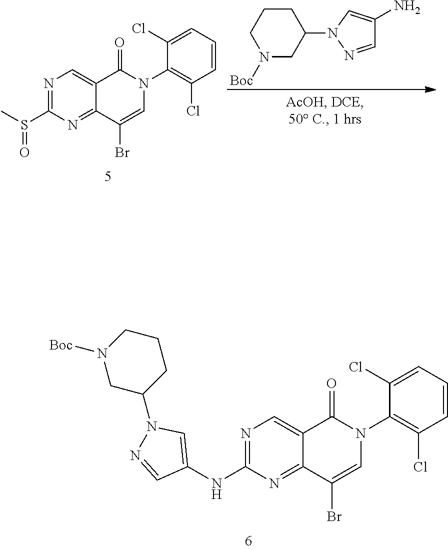

To a solution of 8-bromo-6-(2,6-dichlorophenyl)-2-methylsulfinyl-pyrido[4,3-d]pyrimidin-5-one (62 mg, 143.15 μmol, 1.0 eq.) in DCE (2 mL) was added tert-butyl 3-(4-aminopyrazol-1-yl)piperidine-1-carboxylate (57.19 mg, 214.73 μmol, 1.5 eq.). Then AcOH (42.98 mg, 715.76 μmol, 40.93 μL, 5 eq.) was added. The mixture was stirred at 50° C. for 1 hr. The reaction mixture was concentrated and purified by prep-TLC (SiO₂, petroleum ether: ethyl acetate=1:1) to give tert-butyl 3-[4-[[8-bromo-6-(2,6-dichlorophenyl)-5-oxo-pyrido[4,3-d]pyrimidin-2-yl]amino]pyrazol-1-yl]piperidine-1-carboxylate (90 mg, 141.66 μmol, 98.96% yield) as a yellow oil. ESI [M+H-tBu]=580.1/578.1.

### (v) Preparation of Compound P13

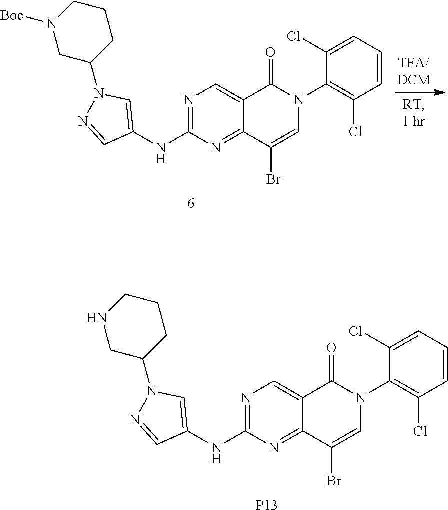

To a solution of tert-butyl 3-[4-[[8-bromo-6-(2,6-dichlorophenyl)-5-oxo-pyrido[4,3-d]pyrimidin-2-yl]amino]pyrazol-1-yl]piperidine-1-carboxylate (90 mg, 141.66 μmol, 1.0 eq.) in DCM (1.5 mL) was added TFA (0.5 mL). The mixture was stirred at 25° C. for 1 hr. The reaction mixture was concentrated and the residue was purified by prep-HPLC (TFA condition, column: Phenomenex Luna 80*30 mm*3 μm; mobile phase: [water(TFA)-ACN]; B %: 10%-40%, 8 min) to give 8-bromo-6-(2,6-dichlorophenyl)-2-[[1-(3-piperidyl)pyrazol-4-yl]amino]pyrido[4,3-d]pyrimidin-5-one (18.99 mg, 29.25 μmol, 54.26% yield, 100% purity, TFA) as a yellow gum. ¹H NMR (400 MHz, DMSO-d₆) δ 10.78 (s, 1H), 9.13 (s, 1H), 8.87 (br s, 1H), 8.46 (s, 1H), 8.37-8.43 (m, 1H), 7.95 (s, 1H), 7.75 (d, J=8.13 Hz, 2H), 7.59-7.65 (m, 1H), 4.53-4.63 (m, 1H), 3.60 (br d, J=8.76 Hz, 1H), 3.24-3.33 (m, 2H), 3.02 (br t, J=9.76 Hz, 1H), 2.22 (br d, J=8.88 Hz, 1H), 1.73-2.10 (m, 4H). ESI [M+H]=535.9/533.9.

### Example 30: Preparation of Compound P12

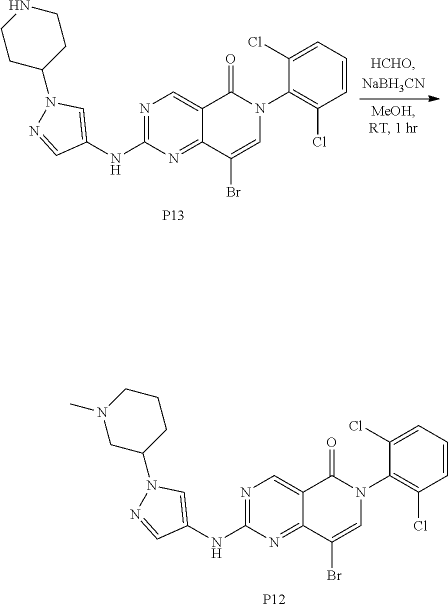

To a solution of 8-bromo-6-(2,6-dichlorophenyl)-2-[[1-(3-piperidyl)pyrazol-4-yl]amino]pyrido[4,3-d]pyrimidin-5-one (35 mg, 65.39 μmol, 1.0 eq.) in MeOH (2 mL) was added DIEA (16.90 mg, 130.79 μmol, 2.0 eq.), and formaldehyde (7.96 mg, 98.09 μmol, 37% purity, 1.5 eq.). The mixture was stirred at 25° C. for 0.25 hr followed by the addition of NaBH₃CN (8.22 mg, 130.79 μmol, 2.0 eq.). The mixture was stirred at 25° C. for 0.75 hr. The reaction mixture was adjusted to pH=7 with sat.aq.Na₂CO₃ and extracted with DCM (5 mL×2). The combined organic layers was dried over Na₂SO₄, filtered and concentrated. The residue was purified by prep-HPLC (neutral condition, column: Waters Xbridge BEH C18 100*30 mm*10 um; mobile phase: [water (NH₄HCO₃)-ACN]; B %: 30%-60%, 10 min) to give 8-bromo-6-(2,6-dichlorophenyl)-2-[[1-(1-methyl-3-piperidyl)pyrazol-4-yl]amino]pyrido[4,3-d]pyrimidin-5-one (10.34 mg, 17.97 μmol, 27.49% yield, 95.48% purity) as a yellow solid. ¹H NMR (400 MHz, DMSO-d₆) δ 10.69 (s, 1H), 9.10 (s, 1H), 8.44 (d, J=3.75 Hz, 2H), 7.82 (s, 1H), 7.75 (d, J=8.00 Hz, 2H), 7.58-7.65 (m, 1H), 4.26-4.35 (m, 1H), 3.31 (s, 1H), 2.99 (br d, J=8.00 Hz, 1H), 2.22 (s, 3H), 1.97-2.05 (m, 2H), 1.57-1.81 (m, 4H). ESI [M+H]=549.9/547.9.

### Example 31

Compounds were tested against two kinases. Compounds were tested in 10-dose IC₅₀ mode with a 3-fold serial dilution starting at 10 μM. Control compound, staurosporine, was tested in 10-dose IC₅₀ mode with 4-fold serial dilution starting at 20 μM. Alternate control compound, Wee-1 Inhibitor, was tested in 10-dose IC₅₀ mode with 3-fold serial dilution starting at 20 μM. Reactions were carried out at 10 μM ATP. See, Tables 2 and 3.

Data includes raw data, % Enzyme activity (relative to DMSO controls) and curve fits.

*Curve fits were performed where the enzyme activities at the highest concentration of compounds were less than 65%.

TABLE 2

| Results of in vitro enzymatic inhibitory activity | | |
|---|---|---|
| of the compounds of the present invention | | |
| Compound | WEE1 IC₅₀ (nM) | PLK1 IC₅₀ |
| AZD1775* | 41 | 159 nM |
| P1 | 41 | 2.55 mM |
| P4 | 47 | 3.53 mM |
| P10 | 27 | 3.42 mM |
| P11 | 31 | 7.04 mM |

TABLE 3

| | Compound IC₅₀* (M) | |
|---|---|---|
| Compound | PLK1 | WEE1 |
| Staurosporine | 1.18 × 10⁻⁷ | ND |
| Wee-1 Inhibitor | ND | 3.02 × 10⁻⁸ |

ND: Indicates compound not tested against enzyme;
Note:
Compound was gently heated to assist dissolution.

### Example 32

In this example, compounds described herein were subject to an assay that detects changes the phases of the cell cycle upon drug treatment, permitting the identification of drugs that affect cell proliferation in a specific manner. For example, problematic DNA replication causes the percentage of cells in S phase to increase. Thus, quantification of cells in S phase permits identification of drugs that cause defects in DNA replication. Similarly, drugs that selectively defects in progression through G2/M can also be identified. Moreover, synergy between assayed drugs and known cell cycle regulators can be determined, as exemplified by the effect of ATR inhibition when combined with agents that slow DNA replication, which causes replication fork collapse into DSBs and complete loss of DNA synthesis. Two novel drugs can also be combined to quantify synergistic effects on specific cell cycle phases.

The assay operates by the quantification of DNA content per cell by flow cytometry. Cells are assessed for effects on cell cycle using a Guava EasyCyte™ flow cytometer (Austin, TX). Drug treated cells are fixed and stained with propidium iodide (PI) and resuspended in phosphate-buffered saline containing the PI staining buffer. During flow cytometry, PI is excited at 528 nm and is detected in a 610/20 bandpass. Readout of the x-axis is the count of cells; readout of the y-axis is optical density of DNA-staining dye. The assay generates a histogram showing count of cells by amount of DNA, which is an indicator of place in the cell cycle. This rapid assay detects relative changes in the cell cycle upon drug treatment upon comparison vehicle-treated and single-agent controls for detection of combinatorial interactions. See, Table 4.

TABLE 4

| | WEE1 Guava MEC | WEE1 Guava MEC |
|---|---|---|
| Compound | Guava MEC | with 20 nM GEM |
| P1 | 0.625 | 0.156 |
| P2 | 5 | 1.25 |
| P3 | 2.5 | 0.625 |
| P4 | 1.25 | 0.625 |
| P5 | >5 | 1.25 |
| P6 | 5 | 1.25 |
| P7 | 2.5 | 1.25 |
| P8 | 5 | 1.25 |
| P9 | 5 | 1.25 |
| P10 | 0.313 | 0.156 |
| P11 | 0.313 | 0.156 |
| P12 | >1.25 | 0.625 |
| P13 | 0.625 | 0.313 |
| P14 | >5 | 1.25 |
| P15 | 5 | 1.25 |
| P16 | 2.5 | 0.625 |
| P17 | 2.5 | 0.625 |
| P19 | 0.313 | 0.156 |
| P20 | 0.313 | 0.156 |
| P21 | 0.625 | 0.313, 0.625 |
| P22 | 2.5 | 1.25 |
| P23 | 5 | 2.5 |
| P24 | 5 | 1.25 |
| P25 | 5 | 0.625 |
| P26 | 5 | 0.625 |
| P27 | 1.25 | 0.625 |

While the invention has been described in detail and with reference to specific examples thereof, it will be apparent to one skilled in the art that various changes and modifications can be made therein without departing from the spirit and scope thereof.

1. A compound of Formula I or III:
(i) a compound of Formula I: 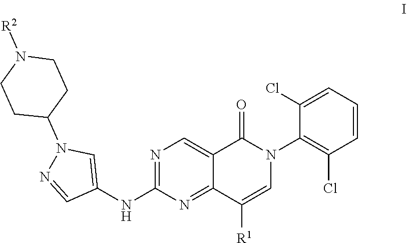
wherein:
R¹ is halo, C₁₋₆ alkyl, C₃₋₈ cycloalkyl or C₂₋₆ alkenyl; and
R² is H, C₁₋₆ alkyl, or C₃₋₈ cycloalkyl;
or a pharmaceutically acceptable salt thereof, or
(ii) a compound of Formula III: 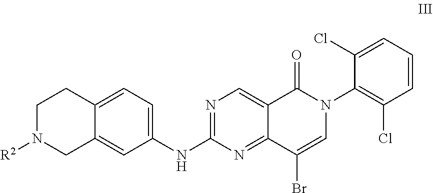
wherein:
R² is H, C₁₋₆alkyl, or C₃₋₈ cycloalkyl;
or a pharmaceutically acceptable salt thereof.

2. The compound of claim 1, wherein R¹ is halo.

3-4. (canceled)

5. The compound of claim 1, wherein R¹ is C₁₋₄ alkyl.

6. (canceled)

7. The compound of claim 1, wherein R¹ is C₃₋₇ cycloalkyl.

8. (canceled)

9. The compound of claim 1, wherein R¹ is C₂₋₄ alkenyl.

10. (canceled)

11. The compound of claim 1, wherein R² is C₁₋₆alkyl.

12-13. (canceled)

14. The compound of claim 1, wherein R² is C₃₋₈cycloalkyl.

15-16. (canceled)

17. The compound of claim 1 that is of Formula II: 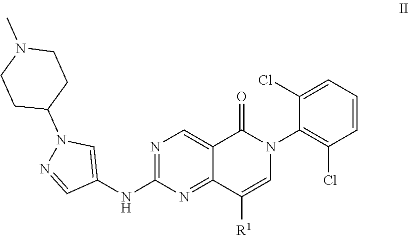
or a pharmaceutically acceptable salt thereof.

18. The compound of claim 1 that is: 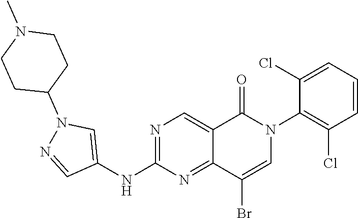 [SMILES: CN1CCC(n2cc(Nc3ncc4c(=O)n(-c5c(Cl)cccc5Cl)cc(Br)c4n3)cn2)CC1]
or a pharmaceutically acceptable salt thereof.

19. The compound of claim 1, that is: 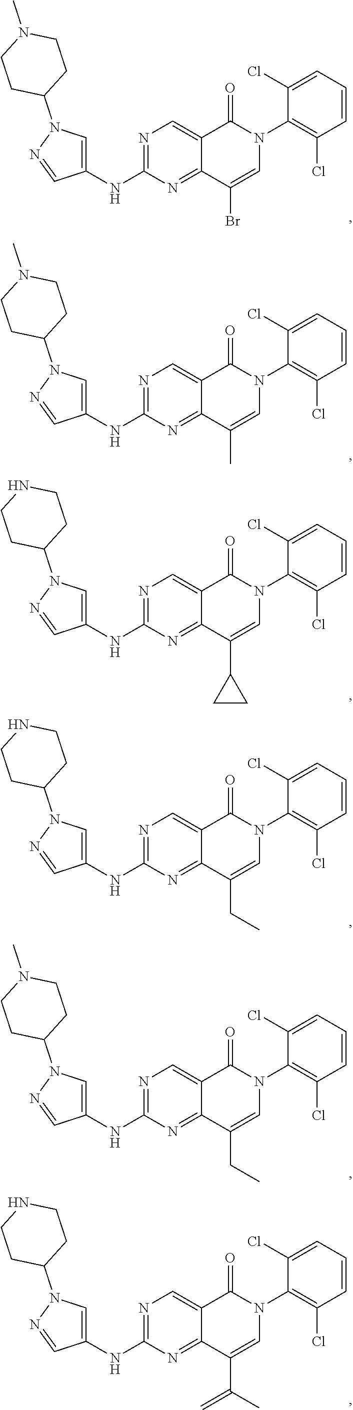 [SMILES: CN1CCC(n2cc(Nc3ncc4c(=O)n(-c5c(Cl)cccc5Cl)cc(Br)c4n3)cn2)CC1 ; Cc1cn(-c2c(Cl)cccc2Cl)c(=O)c2cnc(Nc3cnn(C4CCN(C)CC4)c3)nc12 ; O=c1c2cnc(Nc3cnn(C4CCNCC4)c3)nc2c(C2CC2)cn1-c1c(Cl)cccc1Cl ; CCc1cn(-c2c(Cl)cccc2Cl)c(=O)c2cnc(Nc3cnn(C4CCNCC4)c3)nc12 ; CCc1cn(-c2c(Cl)cccc2Cl)c(=O)c2cnc(Nc3cnn(C4CCN(C)CC4)c3)nc12 ; C=C(C)c1cn(-c2c(Cl)cccc2Cl)c(=O)c2cnc(Nc3cnn(C4CCNCC4)c3)nc12] 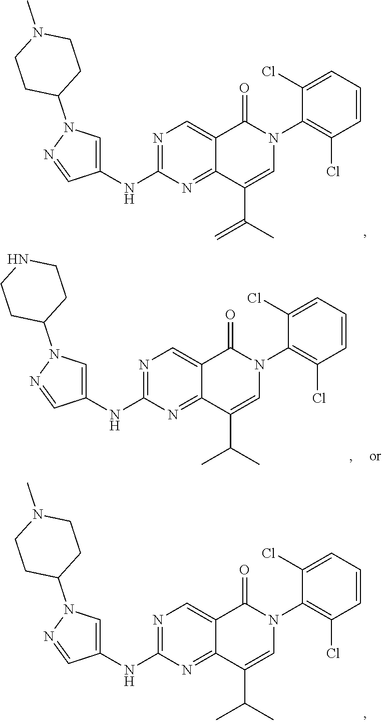 [SMILES: C=C(C)c1cn(-c2c(Cl)cccc2Cl)c(=O)c2cnc(Nc3cnn(C4CCN(C)CC4)c3)nc12 ; CC(C)c1cn(-c2c(Cl)cccc2Cl)c(=O)c2cnc(Nc3cnn(C4CCNCC4)c3)nc12 ; CC(C)c1cn(-c2c(Cl)cccc2Cl)c(=O)c2cnc(Nc3cnn(C4CCN(C)CC4)c3)nc12]
or a pharmaceutically acceptable salt thereof.

20. (canceled)

21. The compound of claim 1, wherein the compound is of Formula III and R² is H.

22-24. (canceled)

25. The compound of claim 1, that is: 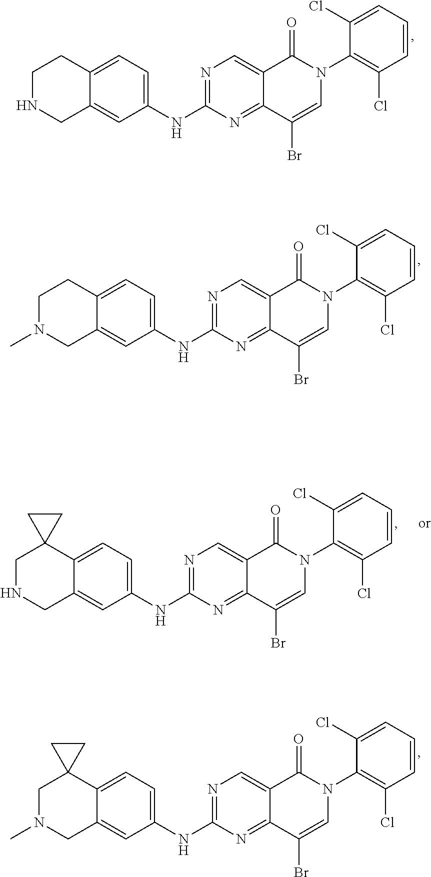 [SMILES: O=c1c2cnc(Nc3ccc4c(c3)CNCC4)nc2c(Br)cn1-c1c(Cl)cccc1Cl ; CN1CCc2ccc(Nc3ncc4c(=O)n(-c5c(Cl)cccc5Cl)cc(Br)c4n3)cc2C1 ; O=c1c2cnc(Nc3ccc4c(c3)CNCC43CC3)nc2c(Br)cn1-c1c(Cl)cccc1Cl ; CN1Cc2cc(Nc3ncc4c(=O)n(-c5c(Cl)cccc5Cl)cc(Br)c4n3)ccc2C2(CC2)C1]
or a pharmaceutically acceptable salt thereof.

26. A compound of Formula IVa, Va, VIa, or VII:
(i) a compound of Formula IVa: 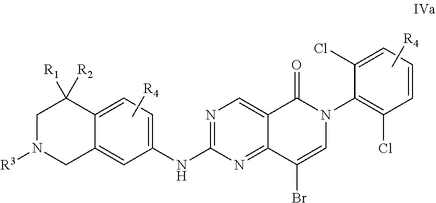
wherein:
R¹ and R² are, independently, H, methyl, ethyl, or propyl; or
R¹ and R² are connected to form a 3-6 membered ring;
R³ is C₁₋₄alkyl or C₃₋₆cycloalkyl;
R⁴ is H, C₁₋₃alkyl, CF₃, —Omethyl, OCF₃, OCF₂H, CN, or halo;
or a pharmaceutically acceptable salt thereof;
(ii) a compound of Formula Va: 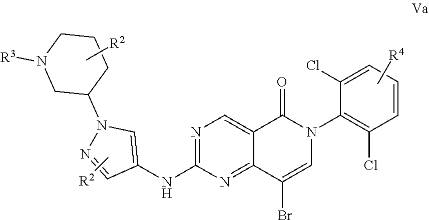
wherein:
R² is H, methyl, ethyl, or propyl;
R³ is C₁₋₄alkyl or C₃₋₆cycloalkyl;
R⁴ is H, C₁₋₃alkyl, CF₃, —Omethyl, OCF₃, OCF₂H, CN, or halo;
or a pharmaceutically acceptable salt thereof;
(iii) a compound of Formula VIa: 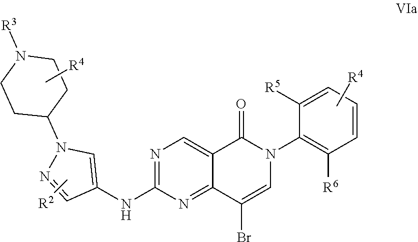
wherein:
R² is H, methyl, ethyl, or propyl;
R³ is C₁₋₄alkyl or C₃₋₆cycloalkyl;
R⁴ is H, C₁₋₃alkyl, CF₃, —Omethyl, OCF₃, OCF₂H, CN, or halo; and
R⁵ and R⁶ are, independently, is H, halo, or C₁₋₆alkyl;
or a pharmaceutically acceptable salt thereof; or
(iv) a compound of Formula VII: 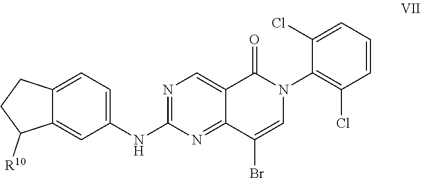
wherein, R¹⁰ is H, OH, NH₂, NH(C₁₋₆alkyl), or N(C₁₋₆alkyl)(C₁₋₆alkyl); or a pharmaceutically acceptable salt thereof.

27. (canceled)

28. The compound of claim 26, that is: 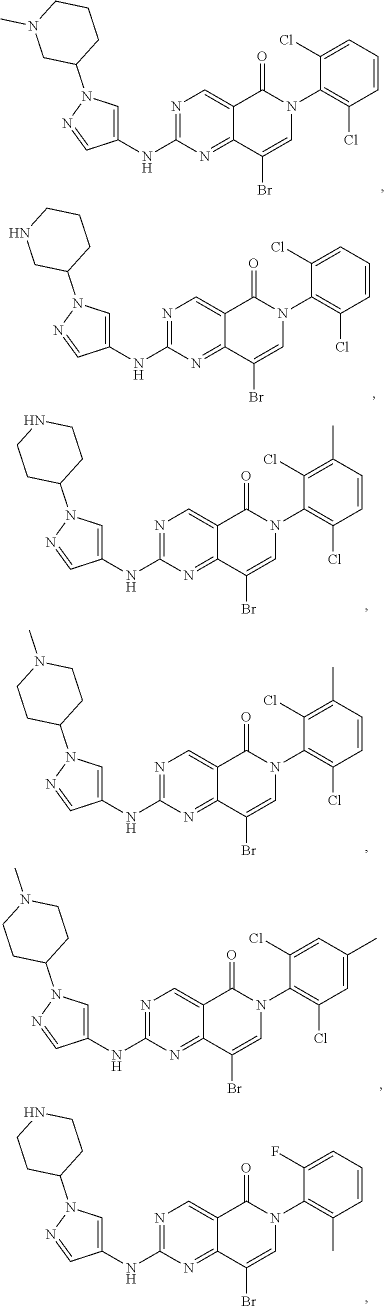 [SMILES: CN1CCCC(n2cc(Nc3ncc4c(=O)n(-c5c(Cl)cccc5Cl)cc(Br)c4n3)cn2)C1 ; O=c1c2cnc(Nc3cnn(C4CCCNC4)c3)nc2c(Br)cn1-c1c(Cl)cccc1Cl ; Cc1ccc(Cl)c(-n2cc(Br)c3nc(Nc4cnn(C5CCNCC5)c4)ncc3c2=O)c1Cl ; Cc1ccc(Cl)c(-n2cc(Br)c3nc(Nc4cnn(C5CCN(C)CC5)c4)ncc3c2=O)c1Cl ; Cc1cc(Cl)c(-n2cc(Br)c3nc(Nc4cnn(C5CCN(C)CC5)c4)ncc3c2=O)c(Cl)c1 ; Cc1cccc(F)c1-n1cc(Br)c2nc(Nc3cnn(C4CCNCC4)c3)ncc2c1=O] 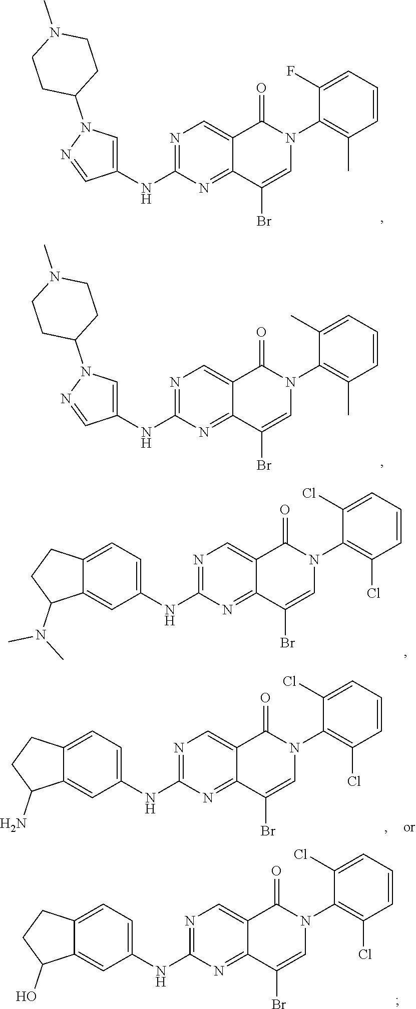
or a pharmaceutically acceptable salt thereof.

29. (canceled)

30. The compound of claim 26, wherein the compound is of Formula VIa and R², R³, and/or R⁵ is H.

31. (canceled)

32. The compound of claim 26, wherein the compound is of Formula VIa and R³, R⁴, R⁵, and/or R⁶ is C₁₋₆alkyl.

33-34. (canceled)

35. The compound of claim 26, wherein the compound is of Formula VIa and R⁵ and/or R⁶ is halo, such as F.

36-40. (canceled)

41. The compound of claim 26, wherein R¹⁰ is H.

42. The compound of claim 26, wherein R¹⁰ is OH.

43. The compound of claim 26, wherein R¹⁰ is NH₂.

44. The compound of claim 26, wherein R¹⁰ is NH(C₁₋₆alkyl).

45. The compound of claim 26, wherein R¹⁰ is N(C₁₋₆alkyl)(C₁₋₆alkyl), such as N(CH₃)₂.

46. (canceled)

47. A pharmaceutical composition comprising one or more compound of claim 1 and one or more pharmaceutically acceptable excipient.

48. A method for inhibiting Wee1, treating cancer, or treating or preventing Wee1-mediated disease in a patient in need of such treatment, comprising administering the compound of claim 1 to the patient.

49. (canceled)

50. The method of claim 48, wherein the cancer is adrenocortical carcinoma, an AIDS-related cancer (such as an AIDS-related lymphoma), anal cancer, appendix cancer, astrocytomas, atypical teratoid/rhabdoid tumor, basal cell carcinoma, bile duct cancer, bladder cancer, bone cancer, brain cancer (such as glioblastoma), breast cancer, bronchial tumor, cancer of unknown primary site such as carcinoma of unknown primary site, carcinoid tumor, castration-resistant prostate cancer, central nervous system cancer (such as central nervous system atypical teratoid/rhabdoid tumor, central nervous system embryonal tumors, central nervous system lymphoma, primary central nervous system lymphoma), cervical cancer, chordoma, chondrosarcoma, chronic myeloproliferative disorders, colon cancer, colorectal cancer, craniopharyngioma, desmoplastic round cell tumor, diffuse large B-cell lymphoma, endometrial cancer, ependymoblastoma, ependymoma, esophageal cancer, Ewing sarcoma family tumor, extracranial germ cell tumor, extragonadal germ cell tumor, extrahepatic bile duct cancer, eye cancer, gallbladder cancer, gastric (stomach) cancer, gastrointestinal carcinoid tumor, gastrointestinal stromal tumor, germ cell tumor, gestational trophoblastic tumor, glioma, head cancer, hepatocellular (liver) cancer, high grade prostate cancer, histiocytosis, hypopharyngeal cancer, Kaposi sarcoma, kidney (renal) cancer, Langerhans cell histiocytosis, laryngeal cancer, leptomeningeal disease, lip cancer, low grade prostate cancer, leukemia (such as chronic lymphocytic leukemia, chronic myelogenous leukemia, hairy cell leukemia,), lung cancer, lymphoma (such as Burkitt lymphoma, central nervous system lymphoma, T-Cell lymphoma such as cutaneous T-Cell lymphoma, Hodgkin's lymphoma, Non-Hodgkin's lymphoma, lymphoplasmacytic lymphoma), medium grade prostate cancer, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, multiple myeloma/plasma cell neoplasm, mycosis fungoides, myelodysplastic syndromes, myelodysplastic/myeloproliferative neoplasms, myeloproliferative disorder, nasal cavity or paranasal sinus cancer, nasopharyngeal cancer, neck cancer, neuroblastoma, ocular cancer, ocular melanoma, oral cancer, oropharyngeal cancer, oral cavity cancer, osteosarcoma or malignant fibrous histiocytoma of bone, osteosarcoma or malignant fibrous histiocytoma, ovarian cancer, ovarian germ cell tumor, ovarian epithelial cancer, ovarian low malignant potential tumor, pancreatic cancer, papillomatosis, paranasal sinus or nasal cavity cancer, parathyroid cancer, penile cancer, pharyngeal cancer, pineal parenchymal tumors of intermediate differentiation, pineoblastoma or supratentorial primitive neuroectodermal tumors, pituitary tumor, pleuropulmonary blastoma, pregnancy cancer, prostate cancer, rectal cancer, renal pelvis cancer, respiratory tract carcinoma involving the NUT gene on chromosome 15, retinoblastoma, rhabdomyosarcoma, salivary gland cancer, sarcoma, Sézary syndrome, skin cancer, skin carcinoma, small intestine cancer, soft tissue sarcoma, spinal cord tumor, squamous cell carcinoma, squamous neck cancer with occult primary, supratentorial primitive neuroectodermal tumors, testicular cancer, throat cancer, thymoma or thymic carcinoma, thyroid cancer, transitional cell cancer of the renal pelvis or ureter, unusual cancers of childhood, ureter cancer, urethral cancer, uterine sarcoma, vaginal cancer, vulvar cancer, Waldenström's macroglobulinemia (lymphoplasmacytic lymphoma), Wilm's tumor, or women's cancer.

51. The method of claim 49, wherein the cancer is breast cancer, prostate cancer, pancreatic cancer, lung cancer, colorectal cancer, ovarian cancer, liver cancer, melanoma, renal cancer, a central nervous system cancer, brain cancer such as glioblastoma, a leukemia, or a lymphoma.

52. The method of claim 49, wherein the compound is administered in combination with at least one additional therapeutic agent such as a chemotherapeutic.

53-54. (canceled)

55. A method for reducing the activity of a kinase encoded by the gene WEE1, comprising contacting the kinase with an inhibitory amount of a compound of claim 1.

56. The method of claim 55, wherein the method is carried out in vitro.

57. The method of claim 55, wherein the method is carried out in a subject.

58-59. (canceled)
